(19) United States
(12) Patent Application Publication
(10) Pub. No.: US 2025/0019350 A1
(43) Pub. Date: Jan. 16, 2025

(54) SYNTHESIS OF THE PROSTATE SPECIFIC MEMBRANE ANTIGEN (PSMA) RADIOLABELED INHIBITOR [18F]DCFPYL

(71) Applicant: Progenics Pharmaceuticals, Inc, New York, NY (US)

(72) Inventors: Pedro Benites, Watertown, MA (US); Simon P. Robinson, Stow, MA (US); Robert W. Siegler, Bedford, MA (US)

(21) Appl. No.: 18/744,219

(22) Filed: Jun. 14, 2024

Related U.S. Application Data
(60) Provisional application No. 63/514,480, filed on Jul. 19, 2023.
(60) Provisional application No. 63/514,485, filed on Jul. 19, 2023.
(60) Provisional application No. 63/508,673, filed on Jun. 16, 2023.

(51) Int. Cl. C07D 213/61

(52) U.S. Cl. CPC C07D 213/61

(57) ABSTRACT

Provided herein are methods and related compositions, including kits, for the improved synthesis, preparation and/or storage of [¹⁸F]DCFPyL. Also provided are methods of using [¹⁸F]DCFPyL, such as for imaging.

### RELATED APPLICATIONS

This application claims the benefit of priority under 35 U.S.C. § 119(e) to U.S. Provisional Application Nos. 63/508,673, filed Jun. 16, 2023; 63/514,480, filed Jul. 19, 2023; and 63/514,485, filed Jul. 19, 2023; the entire contents of each of which are incorporated herein by reference.

### BACKGROUND OF THE INVENTION

With an estimated incidence of over 1 million cases per year and an estimated mortality of 307,000 men per year, prostate cancer is the most common cancer in men and one of the most prevalent cancers worldwide (Mauer, et al., 2016). In the United States alone, there are well over 200 thousand new cases diagnosed annually (Seigel, et al., 2014). Owing in part to serum diagnostic tests for expression of the prostate-specific antigen (PSA) in developing prostate cancer, with proper diagnosis and treatment, the 5-year survival is nearly 99% (seer.cancer.gov).

With greater frequency, the proper diagnosis and monitoring of treatment involves non-invasive molecular imaging. A number of radiotracers for prostate-specific membrane antigen (PSMA) PET imaging for prostate cancer have been developed, including 18F-FACBC (fluciclovine), Ga68 PSMA-11 and DCFPyL. The successful use of DCFPyL (Chen, et al., 2011; Szabo, et al., 2015) and its favorable distribution and imaging characteristics compared to other PSMA targeting radiotracers (Dietlein, et al., 2015) have led to increased demand for this radiotracer.

### SUMMARY OF THE INVENTION

Provided herein are methods and related compositions for the improved synthesis, purification, and/or storage, etc. of [¹⁸F]DCFPyL (also referred to herein as PyL or PYLARIFY). In one aspect, the disclosure provides any one of such methods provided herein.

In another aspect, the disclosure provides any one of the compositions, including kits, as described herein, such as compositions comprising ascorbic acid and [¹⁸F]DCFPyL (or reagents for making [¹⁸F]DCFPyL) as described herein and/or compositions produced by any one of the methods described herein.

In another aspect, the disclosure provides methods of administering to a subject in need thereof any one of the compositions as described herein.

In another aspect, the disclosure provides methods for detecting ¹⁸F, including as a step of any one of the methods provided herein, which method comprises the use of TEAF.

Further provided by this disclosure are kits comprising any one of the compositions as described herein or any one of the compositions as produced by or used in any one of the methods described herein.

### Definitions

As used herein, the term “determining” generally refers to the analysis of molecules or set of molecules or signal or set of signals, for example, quantitatively or qualitatively, and/or the detection of the presence or absence of the molecule or molecules.

The term “diagnostic imaging,” as used herein, refers to a procedure used to detect an imaging agent that can be used in diagnosis of a condition, a disease, and/or a disorder.

The term “diagnosis” as used herein encompasses identification, confirmation, assessment and/or characterization of a condition, a disease, and/or a disorder.

A “kit” comprises a component or a collection of components in one or more containers, such as vials, which can be used by a practicing end user in a clinical or pharmacy setting. A kit provided herein may be used to synthesize a radiopharmaceutical, such as for diagnosis. Thus, a kit may be used by the practicing end user in a clinical or pharmacy setting to synthesize and/or use a radiopharmaceutical in diagnosis. In some embodiments, the kit may provide all the requisite components to synthesize and/or use a radiopharmaceutical except those that are commonly available to the practicing end user, such as water or saline for injection, the radioisotope (e.g., ¹⁸F), equipment for processing the kit during the synthesis and manipulation of the radiopharmaceutical, if required, equipment necessary for administering the radiopharmaceutical to the subject, such as syringes, shielding, imaging equipment, and the like. In some embodiments, imaging agents may be provided to the end user in their final form in a formulation contained typically in one or more containers, such as vials, or one or more syringes, as, for example, an aqueous solution. Thus, is an embodiment, a kit may contain an agent in final form.

As used herein, a “portion of a subject” refers to a particular region of a subject, location of the subject, and the like. For example, a portion of a subject may be the brain, heart, vasculature, cardiac vessels, tumor, etc., of a subject.

As used herein, the term “subject” refers to a human or non-human mammal or animal. Non-human mammals include livestock animals, companion animals, laboratory animals, and non-human primates. Non-human subjects also specifically include, without limitation, horses, cows, pigs, goats, dogs, cats, mice, rats, guinea pigs, gerbils, hamsters, mink, and rabbits. In some embodiments of the invention, a subject is referred to as a “patient.” In some embodiments, a patient or subject may be under the care of a physician or other health care worker, including, but not limited to, someone who has consulted with, received advice from or received a prescription or other recommendation from a physician or other health care worker.

Any of the compounds described herein may be in a variety of forms, such as, but not limited to, salts, solvates, hydrates, tautomers, and isomers. In certain embodiments, the imaging agent is a pharmaceutically acceptable salt of the imaging agent. The term “pharmaceutically acceptable salt” as used herein refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, such description is incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counter ions such as halide, hydroxide, carboxylate, sulfate, phosphate, nitrate, loweralkyl sulfonate and aryl sulfonate.

A “pharmaceutically acceptable carrier” refers to a biocompatible solution, having due regard to sterility, p[Eta], isotonicity, stability, and the like and can include any and all solvents, diluents (including sterile saline, sodium chloride injection, Ringer's injection, dextrose injection, Dextrose and sodium chloride injection, lactated Ringer's injection and other aqueous buffer solutions), dispersion media, coatings, antibacterial and antifungal agents, isotonic agents, and the like. The pharmaceutically acceptable carrier may also contain stabilizers, preservatives, antioxidants, or other additives, which are known to one of skill in the art, or other vehicles as known in the art.

In certain embodiments, the compound is in the form of a hydrate or solvate. The term “hydrate” as used herein refers to a compound non-covalently associated with one or more molecules of water. Likewise, the term “solvate” refers to a compound non-covalently associated with one or more molecules of an organic solvent.

Reference to “about” with respect to a value or parameter herein refers to the usual error range for the respective value or parameter readily known to the skilled person in this technical field. Reference to “about” for a value or parameter herein includes (and describes) aspects that are directed to that value or parameter per se. For example, description referring to “about X” includes description of “X.” In an embodiment of any one of the compositions or methods provided herein, “about” refers to the value or parameter and ±10%, 9%, 8%, 7%, 6%, 5%, 4%, 3%, 2%, or 1%. In an embodiment of any one of the compositions or methods provided herein, any amount recited herein can refer to the amount alone and without “about”.

### BRIEF DESCRIPTION OF THE DRAWINGS

FIG. 1 shows a [¹⁸F]DCFPyL synthesis method.

FIG. 2 shows a chromatogram of [¹⁸F]DCFPyL and free ¹⁸F radiolytic impurity.

FIG. 3 shows a chromatogram of 5 μCi ¹⁸F spiked with TEAF (44 mg/mL).

FIG. 4 shows formation of free ¹⁸F in the presence of ethanol.

FIG. 5 shows a HPLC radiodetector trace of sample with or without a TEAF spike.

FIG. 6 shows a HPLC radiodetector trace of a sample in 35% ethanol in saline.

FIG. 7 shows [¹⁸F]DCFPyL concentration-dependent formation of free ¹⁸F in the presence of 20% ethanol in saline.

FIG. 8 shows a HPLC radiodetector trace of T0 and T4 hr samples from [¹⁸F]DCFPyL in 100 mg/mL ascorbic acid, pH 2.

FIG. 9 shows a HPLC radiodetector trace of T0 and T4 hr samples from [¹⁸F]DCFPyL in 50 mg/mL ascorbic acid, pH 5.8.

FIG. 10 shows [¹⁸F]DCFPyL concentration-dependent formation of free ¹⁸F in the presence of ascorbic acid.

FIG. 11 shows [¹⁸F]DCFPyL concentration-dependent formation of free ¹⁸F in the presence of sodium ascorbate.

FIG. 12 shows a [¹⁸F]DCFPyL drug product production process.

FIG. 13 shows an AIO reagent list and schematic showing reagent positions.

FIG. 14 shows a flow diagram indicating the different points where ascorbic acid was introduced.

FIG. 15 shows T=0 hour radiochromatogram for 147 mCi [¹⁸F]DCFPyL/mL radiolysis of 110 mCi of [¹⁸F]DCFPyL/mL in 50 mg/mL, pH 5.8 ascorbic acid.

FIG. 16 shows a plot of RCP versus time for lots 220816PyL, 220817PyL and 220818PyL.

FIG. 17 shows a plot of ¹⁸F formation versus time for lots 220816PyL, 220817PyL and 220818PyL.

FIG. 18 shows a plot of the formation of the peak at retention time 6.0 minutes versus time for lots 220816PyL, 220817PyL and 220818PyL.

FIG. 19 shows a plot of the formation of the peak at retention time 9.8 minutes versus time for lots 220816PyL, 220817PyL and 220818PyL.

FIG. 20 shows a plot of the formation of the peak at retention time 10.6 minutes versus time for lots 220816PyL, 220817PyL and 220818PyL.

FIG. 21 shows a plot of RCP calculated for inclusion of only the ¹⁸F degradation product versus time for lots 220816PyL, 220817PyL and 220818PyL.

FIG. 22 shows a representative radiochemical chromatogram for [¹⁸F]DCFPyL formulated in 5.0 mg/mL, pH 6, ascorbic acid in saline.

FIG. 23 shows a radiochemical chromatogram for Lot 220908PyL; peak cut from the semi-prep HPLC purification delivered into SWFI in the collection vial then to the FPV containing 0.9% saline, analyzed without further processing (166 mCi/mL).

FIG. 24 shows a T=4 hour radiochemical chromatogram for lot 220909PyL; 5 mg/mL, pH 6 ascorbic acid in collection vial and 10 mg/mL, pH 6 ascorbic acid in 0.9% saline in FPV.

FIG. 25 shows a T=4 hour radiochemical chromatogram for lot 220914PyL; 5 mg/mL, pH 2 ascorbic acid in SWFI in the collection vial; 5 mg/mL, pH 6 ascorbic acid in the saline vial; and 5 mg/mL, pH 6 ascorbic acid in 0.9% saline in the FPV.

FIG. 26 shows a T=4 hour radiochemical chromatogram for lot 220927PyL; 5 mg/mL, pH 2 ascorbic acid in SWFI in the collection vial; 10 mg/mL, pH 6 ascorbic acid in 0.9% saline in the FPV vial.

FIG. 27 shows a T=4 hour radiochemical chromatogram for lot 220928PyL; 5 mg/mL, pH 2 ascorbic acid in SWFI in the collection vial and 10 mg/mL, pH 6 ascorbic acid in the saline vial.

FIG. 28 shows a T=10 hour radiochemical chromatogram for [¹⁸F]DCFPyL (lot 221025PyL) produced with 10 mg/mL, pH 2 ascorbic acid loaded in the collection vial; and 10 mg/mL, pH 5.5 ascorbic acid preloaded in the FPV.

FIG. 29 shows a T=10 hour radiochemical chromatogram for [¹⁸F]DCFPyL (lot 221026PyL) produced with 10 mg/mL, pH 2 ascorbic acid loaded in the collection vial and 10 mg/mL, pH 4.5 ascorbic acid preloaded in the FPV.

FIG. 30 shows a T=10 hour radiochemical chromatogram for [¹⁸F]DCFPyL (lot 230124PyL produced with 10 mg/mL, pH 2 ascorbic acid loaded in the collection vial and 10 mg/mL, pH 7.0 ascorbic acid preloaded in the FPV.

FIG. 31 shows a T=10 hour radiochemical chromatogram for [¹⁸F]DCFPyL (lot 230130PyL) produced with 10 mg/mL, pH 2 ascorbic acid loaded in the collection vial; and 6.6 mg/mL, pH 5.5 ascorbic acid preloaded in the FPV.

FIG. 32 shows a T=10 hour radiochemical chromatogram for [¹⁸F]DCFPyL (lot 230131PyL) produced with 10 mg/mL, pH 2 ascorbic acid loaded in the collection vial; and 15.6 mg/mL, pH 5.5 ascorbic acid preloaded in the FPV.

FIG. 33 shows a lot 220908PyL radiochemical chromatogram; peak cut from the semi-prep HPLC purification delivered into SWFI in the collection vial then to the FPV containing 0.9% saline, analyzed without further processing (115 mCi/mL).

FIG. 34 shows a T=6 hour UV impurity chromatogram of a development lot.

FIG. 35 shows a T=5 day UV impurity chromatogram of a development lot.

FIG. 36 illustrates the generation of ascorbic acid impurity peak over time.

FIG. 37 shows an ascorbic acid matrix solution UV chromatogram with and without F¹⁸ present.

FIG. 38 shows UV-HPLC/MS chromatograms of a development lot. Green highlighted area in mass chromatogram represents background subtraction range. Light blue highlighted area in mass chromatogram represents the retention time range for the unknown impurity (3.6-3.9 min).

FIG. 39 shows UV-HPLC/MS chromatograms of 5 mg/mL ascorbic acid+F¹⁸ solution T=24 hour. Green highlighted area in mass chromatogram represents background subtraction range. Light blue highlighted area in mass chromatogram represents the retention time range for the unknown impurity (3.6-4.0 min).

FIG. 40 shows a flow diagram indicating examples of different points where ascorbic acid can be introduced in a [¹⁸F]-DCFPyL ascorbic acid formulation.

### DETAILED DESCRIPTION OF THE INVENTION

[¹⁸F]DCFPyL (pictured below) has been found to be a powerful imaging agent for the assessment of cancer, such as prostate cancer. However, generally, there is an upper limit of 80 mCi/mL radioactive concentration at end of synthesis (EOS). In order to improve drug supply and/or allow for more doses per lot (lot size may be limited by the maximum drug concentration product vial size (e.g., 50 mL)) and/or improve storage, a product having a higher radioactive concentration, such as >80 mCi/mL at EOS, can be beneficial. In addition, the major radioactive degradation product observed is free ¹⁸F and is formed through radiolysis of [¹⁸F]DCFPyL. Producing [¹⁸F]DCFPyL while minimizing radiolysis can also be beneficial.

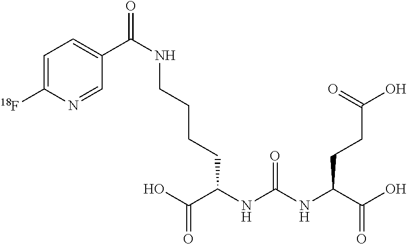 [SMILES: O=C(O)CC[C@H](NC(=O)N[C@@H](CCCCNC(=O)c1ccc([18F])nc1)C(=O)O)C(=O)O]

Provided herein are methods of producing [¹⁸F]DCFPyL that can provide for higher radioactive concentrations and purity with little to no radiolysis, as well as related compositions. These methods and composition can help provide better access to the imaging agent for patients as well as more pure formulations for improved use. Methods provided herein and related compositions include the use of ascorbic acid. Ascorbic acid is a buffering agent with a pKa of 4.2. However, the concentrations and pHs of solutions with ascorbic acid that can result in desirable features, such as maintenance of purity at a desired level of radioactive concentration, for a specific radiopharmaceutical agent, vary and are unique to the specific radiopharmaceutical agent.

Provided herein, are compositions and methods that include the use of ascorbic acid at specific concentrations and pH, such as in the synthesis and/or storage of [¹⁸F]DCFPyL that can result in preparations that have high maintained high purity at high radioactive concentrations as well as other desirable features, which desirable features can be a result not only at end of synthesis (EOS) but also post synthesis, such as 10 hours post synthesis, in an embodiment of any one of the methods or compositions provided herein. Such features can be important for the availability and use of [¹⁸F]DCFPyL in imaging patients.

It has been surprisingly found that certain concentrations of ascorbic acid within certain pH ranges are particularly beneficial in the production [¹⁸F]DCFPyL. The invention is generally directed to at least methods of preparing or synthesizing compositions comprising [¹⁸F]DCFPyL with ascorbic acid at such concentrations and within such pH ranges, as well as related compositions and methods. The disclosure advantageously provides [¹⁸F]DCFPyL formulations which utilize ascorbic acid as a stabilizer that can have increased radioactive concentrations and purity of [¹⁸F]DCFPyL. In addition, the pH ranges can enhance the stability and shelf-life of the compositions and can also minimize severe localized site reactions upon injection, in some embodiments. In some embodiments, it has been found that beneficial features of formulations made with the methods provided herein can be found at end of synthesis of [¹⁸F]DCFPyL as well as well post synthesis, such as 10 hours post synthesis. Thus, in some embodiments, ascorbic acid at the concentrations provided herein and within certain pH ranges can serve as a stabilizer during preparation, transport and/or storage of the radiopharmaceutical compositions of [¹⁸F]DCFPyL with surprisingly beneficial results.

Ascorbic acid is utilized as a stabilizing component of the [¹⁸F]DCFPyL radiopharmaceutical compositions provided herein. Ascorbic acid is known as vitamin C and has been used as an antioxidant to prevent radiolytic decomposition of certain radiopharmaceuticals (WO95/33757; Anticancer Res. 1997, 17, 1783-1796; U.S. Pat. Nos. 5,093,105, and 5,306,482) or radiolabeled peptides (U.S. Pat. Nos. 5,393,512; 5,384,113 and 5,961,955). As used herein, the term “ascorbic acid” includes ascorbic acid itself as well as analogs and salts of the acid known to those of ordinary skill in the art. Ascorbic acid is a readily available FDA inactive ingredient and can be used in pharmaceutical compositions and other formulations used for biological purposes, at levels as high as 200 mg/mL of the final formulation. Previous compositions including ascorbic acid were typically at pH values within biological pH range (e.g., 6-8) during essentially all processing steps, as well as administration to a subject, to reduce the risk of irritation and pain associated with acidic solutions. However, within biological pH range, the ability of ascorbic acid/ascorbate in buffered solutions to stabilize radiopharmaceutical solutions can be surprisingly reduced.

Ascorbic acid as described herein has been found to be a highly effective protectant for PyL. It is important to note that different compounds can be protected differently by different protectants or stabilizers. In addition, for any particular compound, concentrations and pH that are effective for a possible protectant can also vary, including in combination with other components of a composition, such as ethanol, concentrations for which can also vary. For example, ethanol in FDG can stabilize de-fluorination at very low concentrations (Dantas et al 2012, Fawdry 2007), while ethanol in some other products can stabilize in 100% EtoH or 50% ethanol (Scott 2009 AV 19). As another example, ascorbic acid can stabilize FDG from de-fluorination but at 10 mg/mL to get less than 2% @ 10 hr (Fawdry 2007). Ascorbic acid has a pH dependency for the inhibition of radiolytic processes (Castner et al U.S. Pat. No. 9,687,571), so the amount of ascorbic acid needed to radioprotect is compound-dependent and pH-dependent and was not known for PyL until the discoveries described herein were made. Further, the detection of impurities at a level as described herein using HPLC was also not known or recognized.

Some advantages of using ascorbic acid or its analogs in a [¹⁸F]DCFPyL radiopharmaceutical composition disclosed herein include: (1) the ability to prepare radiopharmaceutical compositions at radioactive concentrations of at least 80 mCi/mL; (2) the ability to prepare radiopharmaceutical compositions with a radiochemical purity of at least 90% at 10 hours post synthesis; and/or (3) the ability to prepare radiopharmaceutical compositions that have less than 5% free ¹⁸F. In some embodiments of any one of the compositions or methods provided herein, ascorbate salts may be added to the formulation. In some embodiments of any one of the compositions or methods provided herein, ascorbic acid may be used in the uncharged form, or in compositions in which a higher percentage of ascorbic acid is protonated at the appropriate pH. Without being bound by any particular theory, the efficacy of the antioxidant, in some cases, may be directly related to the non-ionic nature of the hydrogen-oxygen bonds in the antioxidant, with enhanced stability at acidity levels wherein a significant portion of the antioxidant is in protonated form.

In this and any one of the other aspects and embodiments of the invention, ascorbic acid may be present in an acidic form (e.g., as ascorbic acid) and/or basic form (e.g., as ascorbate), depending on pH. For example, at pH values greater than about 4.2 (i.e., the pKa of ascorbic acid), the basic form will be more prevalent than the acidic form. The higher the pH, the higher the proportion that is present as the basic form. Conversely, at pH values less than about 4.2, the acidic form will be more prevalent than the basic form. The lower the pH, the higher the proportion that is present as the acidic form. Accordingly, where the term ascorbic acid is used herein in connection with a composition, it should be understood that the composition may comprise the acidic form of ascorbic acid, the basic form of ascorbic acid, or combinations thereof.

In some embodiments of any one of the methods or compositions provided herein, the basic form (i.e., ascorbate) may be associated with a counter ion. Those of ordinary skill in the art will be aware of pharmaceutically acceptable salts suitable for association with ascorbate and for use with the compositions described herein. Non-limiting examples of pharmaceutically acceptable salts are described herein. In some cases of any one of the methods or compositions provided herein, the counter ion is sodium (e.g., such that the composition comprises sodium ascorbate).

In some embodiments of any one of the methods or compositions provided herein, the radiopharmaceutical compositions provided herein may include ascorbic acid as a stabilizer, in the absence of other stabilizers compounds. In some embodiments of any one of the methods or compositions provided herein, the radiopharmaceutical compositions provided herein may include ascorbic acid as a stabilizer as well as ethanol. In some of these embodiments, the ethanol is maintained at any one of the specific concentrations as provided herein.

In some embodiments of any one of the methods or compositions provided herein, the pH of the composition is about 2.5, about 2.6, about 2.7, about 2.8, about 2.9, about 3, about 3.1, about 3.2, about 3.3, about 3.4, about 3.5, about 4, about 4.5, about 5, about 5.5, about 6, about 6.5, about 7, about 7.1, about 7.2, about 7.3, about 7.4, about 7.5, about 7.6, about 7.7, about 7.8, about 7.9 or about 8. In some embodiments of any one of the methods or compositions provided herein, the pH of the composition is between about 2.5 and about 8. In some embodiments of any one of the methods or compositions provided herein, the pH of the composition is between about 3 and about 8. In some embodiments of any one of the methods or compositions provided herein, the pH of the composition is between about 3 and about 7.5. In some embodiments of any one of the methods or compositions provided herein, the pH of the composition is between about 3.3 and about 7.8. In some embodiments of any one of the methods or compositions provided herein, the pH of the composition is between about 3.5 and about 7.5. In some embodiments of any one of the methods or compositions provided herein, the pH of the composition is between about 4 and about 7.5. In some embodiments of any one of the methods or compositions provided herein, the pH of the composition is between about 4.5 and about 7.5. In some embodiments of any one of the methods or compositions provided herein, the pH of the composition is between about 5 and about 7.5. In some embodiments of any one of the methods or compositions provided herein, the pH of the composition is between about 2.5 and about 7. In some embodiments of any one of the methods or compositions provided herein, the pH of the composition is between about 3 and about 7. In some embodiments of any one of the methods or compositions provided herein, the pH of the composition is between about 3.5 and about 7. In some embodiments of any one of the methods or compositions provided herein, the pH of the composition is between about 4 and about 7. In some embodiments of any one of the methods or compositions provided herein, the pH of the composition is between about 4.5 and about 7. In some embodiments of any one of the methods or compositions provided herein, the pH of the composition is between about 5 and about 7. In some embodiments of any one of the methods or compositions provided herein, the pH of the composition is between about 5.5 and about 7. In some embodiments of any one of the methods or compositions provided herein, the pH of the composition is between about 2.5 and about 6.5. In some embodiments of any one of the methods or compositions provided herein, the pH of the composition is between about 3 and about 6.5. In some embodiments of any one of the methods or compositions provided herein, the pH of the composition is between about 3.5 and about 6.5. In some embodiments of any one of the methods or compositions provided herein, the pH of the composition is between about 4 and about 6.5. In some embodiments of any one of the methods or compositions provided herein, the pH of the composition is between about 4.5 and about 6.5. In some embodiments of any one of the methods or compositions provided herein, the pH of the composition is between about 5 and about 6.5. In some embodiments of any one of the methods or compositions provided herein, the pH of the composition is between about 5.5 and about 6.5. In some embodiments of any one of the methods or compositions provided herein, the pH of the composition is between about 2.5 and about 6. In some embodiments of any one of the methods or compositions provided herein, the pH of the composition is between about 3 and about 6. In some embodiments of any one of the methods or compositions provided herein, the pH of the composition is between about 3.5 and about 6. In some embodiments of any one of the methods or compositions provided herein, the pH of the composition is between about 4 and about 6. In some embodiments of any one of the methods or compositions provided herein, the pH of the composition is between about 4.5 and about 6. In some embodiments of any one of the methods or compositions provided herein, the pH of the composition is between about 5 and about 6. In some embodiments of any one of the methods or compositions provided herein, the pH of the composition is between about 5.5 and about 6. In some embodiments of any one of the methods or compositions provided herein, the pH of the composition is between about 2.5 and about 5.5. In some embodiments of any one of the methods or compositions provided herein, the pH of the composition is between about 3 and about 5.5. In some embodiments of any one of the methods or compositions provided herein, the pH of the composition is between about 3.5 and about 5.5. In some embodiments of any one of the methods or compositions provided herein, the pH of the composition is between about 4 and about 5.5. In some embodiments of any one of the methods or compositions provided herein, the pH of the composition is between about 4.5 and about 5.5. In some embodiments of any one of the methods or compositions provided herein, the pH of the composition is between about 5 and about 5.5. In some embodiments of any one of the methods or compositions provided herein, the pH is any one of the pHs provided herein.

In some embodiments of any one of the methods or compositions provided herein, the pH of the composition is about 2.5, about 3.0, about 3.5, about 4.0, about 4.5, about 5.0, about 5.5, about 6.0, about 6.5, about 7.0, about 7.5 or about 8.0. In some embodiments of any one of the methods or compositions provided herein, the pH of the composition is between about 2.5 and about 7.5. In some embodiments of any one of the methods or compositions provided herein, the pH of the composition is between about 3.0 and about 7.5. In some embodiments of any one of the methods or compositions provided herein, the pH of the composition is between about 3.5 and about 7.5. In some embodiments of any one of the methods or compositions provided herein, the pH of the composition is between about 4.0 and about 7.5. In some embodiments of any one of the methods or compositions provided herein, the pH of the composition is between about 4.5 and about 7.5. In some embodiments of any one of the methods or compositions provided herein, the pH of the composition is between about 5.0 and about 7.5. In some embodiments of any one of the methods or compositions provided herein, the pH of the composition is between about 2.5 and about 7.0. In some embodiments of any one of the methods or compositions provided herein, the pH of the composition is between about 3.0 and about 7.0. In some embodiments of any one of the methods or compositions provided herein, the pH of the composition is between about 3.5 and about 7.0. In some embodiments of any one of the methods or compositions provided herein, the pH of the composition is between about 4.0 and about 7.0. In some embodiments of any one of the methods or compositions provided herein, the pH of the composition is between about 4.5 and about 7.0. In some embodiments of any one of the methods or compositions provided herein, the pH of the composition is between about 5.0 and about 7.0. In some embodiments of any one of the methods or compositions provided herein, the pH of the composition is between about 5.5 and about 7.0. In some embodiments of any one of the methods or compositions provided herein, the pH of the composition is between about 2.5 and about 6.5. In some embodiments of any one of the methods or compositions provided herein, the pH of the composition is between about 3.0 and about 6.5. In some embodiments of any one of the methods or compositions provided herein, the pH of the composition is between about 3.5 and about 6.5. In some embodiments of any one of the methods or compositions provided herein, the pH of the composition is between about 4.0 and about 6.5. In some embodiments of any one of the methods or compositions provided herein, the pH of the composition is between about 4.5 and about 6.5. In some embodiments of any one of the methods or compositions provided herein, the pH of the composition is between about 5.0 and about 6.5. In some embodiments of any one of the methods or compositions provided herein, the pH of the composition is between about 5.5 and about 6.5. In some embodiments of any one of the methods or compositions provided herein, the pH of the composition is between about 2.5 and about 6.0. In some embodiments of any one of the methods or compositions provided herein, the pH of the composition is between about 3.0 and about 6.0. In some embodiments of any one of the methods or compositions provided herein, the pH of the composition is between about 3.5 and about 6.0. In some embodiments of any one of the methods or compositions provided herein, the pH of the composition is between about 4.0 and about 6.0. In some embodiments of any one of the methods or compositions provided herein, the pH of the composition is between about 4.5 and about 6.0. In some embodiments of any one of the methods or compositions provided herein, the pH of the composition is between about 5.0 and about 6.0. In some embodiments of any one of the methods or compositions provided herein, the pH of the composition is between about 5.5 and about 6.0. In some embodiments of any one of the methods or compositions provided herein, the pH of the composition is between about 2.5 and about 5.5. In some embodiments of any one of the methods or compositions provided herein, the pH of the composition is between about 3.0 and about 5.5. In some embodiments of any one of the methods or compositions provided herein, the pH of the composition is between about 3.5 and about 5.5. In some embodiments of any one of the methods or compositions provided herein, the pH of the composition is between about 4.0 and about 5.5. In some embodiments of any one of the methods or compositions provided herein, the pH of the composition is between about 4.5 and about 5.5. In some embodiments of any one of the methods or compositions provided herein, the pH of the composition is between about 5.0 and about 5.5.

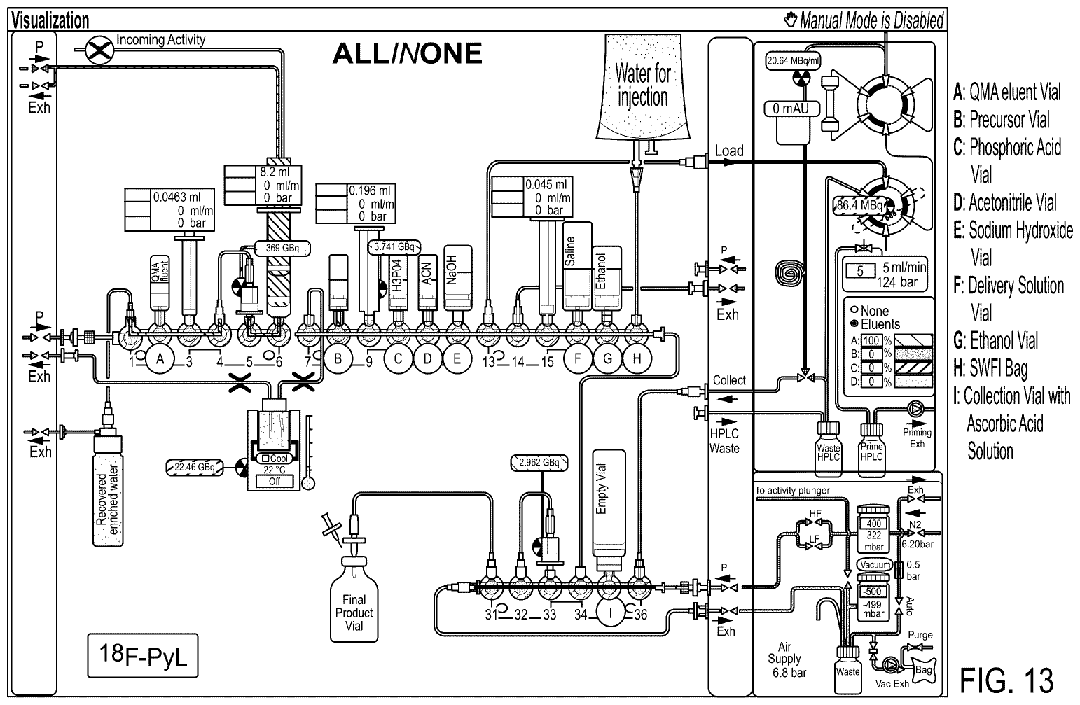

In one embodiment of any one of the foregoing pHs, the pH is of the solution that is in a collection vial, such as a final collection vial (FCV). In one embodiment of any one of the compositions or methods provided herein, the collection vial is after a semi-preparation (e.g., semi-prep with HPLC) but before solvent exchange. As used herein, the FCV refers to a vial that collects a solution comprising [¹⁸F]DCFPyL that has been prepared or synthesized such that the solution is one deemed to be the resulting solution of a preparation or synthesis method, such as a method provided herein. In one embodiment of any one of the methods or compositions provided herein, the solution collected in a FCV is one that can be administered to a subject without further manipulation except for dilution. In one embodiment of any one of the compositions or methods provided herein, the collection vial is a Final Product Vial (FPV). In one embodiment of any one of the compositions or methods provided herein, the FPV is after solvent exchange and is ready for administration (e.g., FIG. 13).

In some embodiments of any one of the methods or compositions provided herein, ascorbic acid is present in a concentration that is about 2 mg/mL, about 2.5 mg/mL, about 3.3 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL, about 10 mg/mL or about 15 mg/mL. In some embodiments of any one of the methods or compositions provided herein, the ascorbic acid concentration of the composition is between about 2.5 and about 7.5, about 3.5 and about 7.5, about 5 and about 7.5, about 2.5 and about 10, about 3.5 and about 10, about 5 and about 10, about 2.5 and about 15, about 3.5 and about 15, or about 5 and about 15 mg/mL. In some embodiments of any one of the methods or compositions provided herein, the ascorbic acid concentration of the composition is between about 2.5 and about 7.5 mg/mL. In some embodiments of any one of the methods or compositions provided herein, the ascorbic acid concentration of the composition is between about 3 and about 7.5 mg/mL. In some embodiments of any one of the methods or compositions provided herein, the ascorbic acid concentration of the composition is between about 3.0 and about 7.5 mg/mL. In some embodiments of any one of the methods or compositions provided herein, the ascorbic acid concentration of the composition is between about 3.3 and about 7.8 mg/mL. In some embodiments of any one of the methods or compositions provided herein, the ascorbic acid concentration of the composition is between about 3.5 and about 7.5 mg/mL. In some embodiments of any one of the methods or compositions provided herein, the ascorbic acid concentration of the composition is between about 4 and about 7.5 mg/mL. In some embodiments of any one of the methods or compositions provided herein, the ascorbic acid concentration of the composition is between about 4.0 and about 7.5 mg/mL. In some embodiments of any one of the methods or compositions provided herein, the ascorbic acid concentration of the composition is between about 4.5 and about 7.5 mg/mL. In some embodiments of any one of the methods or compositions provided herein, the ascorbic acid concentration of the composition is between about 5 and about 7.5 mg/mL. In some embodiments of any one of the methods or compositions provided herein, the ascorbic acid concentration of the composition is between about 5.0 and about 7.5 mg/mL. In some embodiments of any one of the methods or compositions provided herein, the ascorbic acid concentration of the composition is between about 2.5 and about 7 mg/mL. In some embodiments of any one of the methods or compositions provided herein, the ascorbic acid concentration of the composition is between about 2.5 and about 7.0 mg/mL. In some embodiments of any one of the methods or compositions provided herein, the ascorbic acid concentration of the composition is between about 3 and about 7 mg/mL. In some embodiments of any one of the methods or compositions provided herein, the ascorbic acid concentration of the composition is between about 3.0 and about 7.0 mg/mL. In some embodiments of any one of the methods or compositions provided herein, the ascorbic acid concentration of the composition is between about 3.5 and about 7 mg/mL. In some embodiments of any one of the methods or compositions provided herein, the ascorbic acid concentration of the composition is between about 3.5 and about 7.0 mg/mL. In some embodiments of any one of the methods or compositions provided herein, the ascorbic acid concentration of the composition is between about 4 and about 7 mg/mL. In some embodiments of any one of the methods or compositions provided herein, the ascorbic acid concentration of the composition is between about 4.0 and about 7.0 mg/mL. In some embodiments of any one of the methods or compositions provided herein, the ascorbic acid concentration of the composition is between about 4.5 and about 7 mg/mL. In some embodiments of any one of the methods or compositions provided herein, the ascorbic acid concentration of the composition is between about 4.5 and about 7.0 mg/mL. In some embodiments of any one of the methods or compositions provided herein, the ascorbic acid concentration of the composition is between about 5 and about 7 mg/mL. In some embodiments of any one of the methods or compositions provided herein, the ascorbic acid concentration of the composition is between about 5.0 and about 7.0 mg/mL. In some embodiments of any one of the methods or compositions provided herein, the ascorbic acid concentration of the composition is between about 5.5 and about 7 mg/mL. In some embodiments of any one of the methods or compositions provided herein, the ascorbic acid concentration of the composition is between about 5.5 and about 7.0 mg/mL. In some embodiments of any one of the methods or compositions provided herein, the ascorbic acid concentration of the composition is between about 2.5 and about 6.5 mg/mL. In some embodiments of any one of the methods or compositions provided herein, the ascorbic acid concentration of the composition is between about 3 and about 6.5 mg/mL. In some embodiments of any one of the methods or compositions provided herein, the ascorbic acid concentration of the composition is between about 3.0 and about 6.5 mg/mL. In some embodiments of any one of the methods or compositions provided herein, the ascorbic acid concentration of the composition is between about 3.5 and about 6.5 mg/mL. In some embodiments of any one of the methods or compositions provided herein, the ascorbic acid concentration of the composition is between about 4 and about 6.5 mg/mL. In some embodiments of any one of the methods or compositions provided herein, the ascorbic acid concentration of the composition is between about 4.0 and about 6.5 mg/mL. In some embodiments of any one of the methods or compositions provided herein, the ascorbic acid concentration of the composition is between about 4.5 and about 6.5 mg/mL. In some embodiments of any one of the methods or compositions provided herein, the ascorbic acid concentration of the composition is between about 5 and about 6.5 mg/mL. In some embodiments of any one of the methods or compositions provided herein, the ascorbic acid concentration of the composition is between about 5.0 and about 6.5 mg/mL. In some embodiments of any one of the methods or compositions provided herein, the ascorbic acid concentration of the composition is between about 5.5 and about 6.5 mg/mL. In some embodiments of any one of the methods or compositions provided herein, the ascorbic acid concentration of the composition is between about 2.5 and about 6 mg/mL. In some embodiments of any one of the methods or compositions provided herein, the ascorbic acid concentration of the composition is between about 2.5 and about 6.0 mg/mL. In some embodiments of any one of the methods or compositions provided herein, the ascorbic acid concentration of the composition is between about 3 and about 6 mg/mL. In some embodiments of any one of the methods or compositions provided herein, the ascorbic acid concentration of the composition is between about 3.0 and about 6.0 mg/mL. In some embodiments of any one of the methods or compositions provided herein, the ascorbic acid concentration of the composition is between about 3.5 and about 6 mg/mL. In some embodiments of any one of the methods or compositions provided herein, the ascorbic acid concentration of the composition is between about 3.5 and about 6.0 mg/mL. In some embodiments of any one of the methods or compositions provided herein, the ascorbic acid concentration of the composition is between about 4 and about 6 mg/mL. In some embodiments of any one of the methods or compositions provided herein, the ascorbic acid concentration of the composition is between about 4.0 and about 6.0 mg/mL. In some embodiments of any one of the methods or compositions provided herein, the ascorbic acid concentration of the composition is between about 4.5 and about 6 mg/mL. In some embodiments of any one of the methods or compositions provided herein, the ascorbic acid concentration of the composition is between about 4.5 and about 6.0 mg/mL. In some embodiments of any one of the methods or compositions provided herein, the ascorbic acid concentration of the composition is between about 5 and about 6 mg/mL. In some embodiments of any one of the methods or compositions provided herein, the ascorbic acid concentration of the composition is between about 5.0 and about 6.0 mg/mL. In some embodiments of any one of the methods or compositions provided herein, the ascorbic acid concentration of the composition is between about 5.5 and about 6 mg/mL. In some embodiments of any one of the methods or compositions provided herein, the ascorbic acid concentration of the composition is between about 5.5 and about 6.0 mg/mL. In some embodiments of any one of the methods or compositions provided herein, the ascorbic acid concentration of the composition is between about 2.5 and about 5.5 mg/mL. In some embodiments of any one of the methods or compositions provided herein, the ascorbic acid concentration of the composition is between about 3 and about 5.5 mg/mL. In some embodiments of any one of the methods or compositions provided herein, the ascorbic acid concentration of the composition is between about 3.0 and about 5.5 mg/mL. In some embodiments of any one of the methods or compositions provided herein, the ascorbic acid concentration of the composition is between about 3.5 and about 5.5 mg/mL. In some embodiments of any one of the methods or compositions provided herein, the ascorbic acid concentration of the composition is between about 4 and about 5.5 mg/mL. In some embodiments of any one of the methods or compositions provided herein, the ascorbic acid concentration of the composition is between about 4.0 and about 5.5 mg/mL. In some embodiments of any one of the methods or compositions provided herein, the ascorbic acid concentration of the composition is between about 4.5 and about 5.5 mg/mL. In some embodiments of any one of the methods or compositions provided herein, the ascorbic acid concentration of the composition is between about 5 and about 5.5 mg/mL. In some embodiments of any one of the methods or compositions provided herein, the ascorbic acid concentration of the composition is between about 5.0 and about 5.5 mg/mL. In some embodiments of any one of the methods or compositions provided herein, the ascorbic acid concentration is any one of the concentrations provided herein.

In one embodiment of any one of the foregoing ascorbic acid concentrations, the ascorbic acid concentration is of the solution that is in a collection vial, such as a FCV or FPV.

In one aspect are methods of preparing a composition comprising [¹⁸F]DCFPyL of the formula:

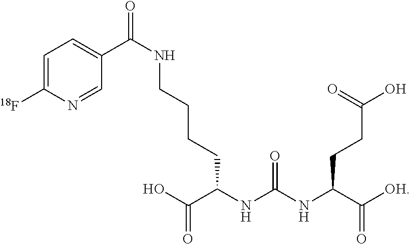 [SMILES: O=C(O)CC[C@H](NC(=O)N[C@@H](CCCCNC(=O)c1ccc([18F])nc1)C(=O)O)C(=O)O]

In one embodiment, such a method comprises adding to a first solution comprising [¹⁸F]DCFPyL a second solution comprising ascorbic acid to form a third solution comprising the [¹⁸F]DCFPyL and ascorbic acid. The concentration of the ascorbic acid and the pH of the second solution may be any one of the concentrations and pHs, respectively, provided herein. Alternatively, the concentration of the ascorbic acid and the pH of the second solution is such that the third solution has any one of the concentrations of ascorbic acid and pHs, respectively, provided herein. Further alternatively, a method of preparing a composition comprising [¹⁸F]DCFPyL may comprise adjusting a solution comprising [¹⁸F]DCFPyL such that the solution has any one of the concentrations of ascorbic acid and pHs, respectively, provided herein. The resulting solution from any one of the foregoing methods may be one that is collected in or transferred to a collection vial, such as a FCV or FPV. The specific concentrations of ascorbic acid and pHs, as provided herein, have been found to result a composition comprising [¹⁸F]DCFPyL with one or more or all of the desirable features provided herein.

In some embodiments, the [¹⁸F]DCFPyL is purified by chromatography (and any one of the methods provided herein can further comprise such purification), prior to addition of the first solution to the second solution or adjusting of the solution as provided herein. In some embodiments of any one of the methods or compositions provided herein, the [¹⁸F]DCFPyL is not purified by chromatography, prior to addition of the first solution to the second solution or adjusting of the solution.

In some embodiments of any one of the methods or compositions provided herein, a solution, such as a first solution, further comprises a solvent, such as one eluted off of a cartridge such as a HLB cartridge. [¹⁸F]DCFPyL and/or the ascorbic acid may be substantially soluble in the solvent. In some embodiments of any one of the methods or compositions provided herein, the composition comprises water. In some embodiments of any one of the methods or compositions provided herein, the composition comprises water and at least one additional solvent, wherein the solvent may be substantially miscible with the water. Non-limiting examples of solvents include, but are not limited to, alcohol solvents (e.g., ethanol, methanol, propanol, isopropanol, tert-butanol). Other non-limiting examples of solvents include acetone, acetic acid, formic acid, dimethyl sulfoxide, dimethyl formamide, acetonitrile, glycol, triethylamine, picoline, and pyridine. In some embodiments of any one of the methods or compositions provided herein, the composition comprises water and a polar solvent substantially miscible with the water.

In some embodiments of any one of the methods or compositions provided herein, the solvent comprises ethanol. In some embodiments of any one of the methods or compositions provided herein, such as where the solvent is eluted or for elution from a cartridge, such as a HLB cartridge, the solution comprises at least about 90%, 91%, 92%, 93%, 94%, 95%, 96%, 97%, 98%, or 99% ethanol (w/v). In some embodiments of the foregoing, the solution comprises 100% ethanol (w/v).

In some embodiments of any one of the methods or compositions provided herein, such as where the solvent is in a collection vial, such as a FCV or FPV, the solution comprises less than or equal to about 30% ethanol (w/v). In some embodiments of any one of the methods or compositions provided herein, the solution comprises less than or equal to about 25% ethanol (w/v). In some embodiments of any one of the methods or compositions provided herein, the solution comprises less than or equal to about 20% ethanol (w/v). In some embodiments of any one of the methods or compositions provided herein, the solution comprises less than or equal to about 15% ethanol (w/v). In some embodiments of any one of the methods or compositions provided herein, the solution comprises less than or equal to about 10% ethanol (w/v). In some embodiments of any one of the methods or compositions provided herein, the solution comprises less than or equal to about 5% ethanol (w/v). In some embodiments of any one of the methods or compositions provided herein, the solution comprises less than or equal to about 3% ethanol (w/v). In one embodiment of any one of the foregoing, the solution comprises at least 3% ethanol (w/v). In some embodiments of any one of the methods or compositions provided herein, the solution comprises ethanol (w/v) at any one of the values provided herein.

In some embodiments of any one of the methods or compositions provided herein, the composition comprises acetonitrile. In some embodiments of any one of the methods or compositions provided herein, such as a solution that is in or transferred to a collection vial, such as a FCV or FPV, the acetonitrile is present at a level that is no more than about 0.05% (w/v), no more than about 0.04% (w/v), no more than about 0.03% (w/v), no more than about 0.02% (w/v) or no more than about 0.01% (w/v). In some embodiments of any one of the methods or compositions provided herein, the solution comprises acetonitrile (w/v) at any one of the values provided herein.

In some embodiments, any one of the methods provided herein can comprise or further comprise steps of applying any one of the solutions provided herein to a solvent exchange cartridge and/or eluting with any one of the solutions provided herein from a solvent exchange cartridge. In one embodiment, any one of the solutions with any one of the ascorbic acid concentrations and any one of the pHs provided herein is applied to the solvent exchange cartridge, such as a HLB cartridge. In another embodiment, any one of the solutions comprising [¹⁸F]DCFPyL is adjusted to have any one of the ascorbic acid concentrations and any one of the pHs provided herein and then applied to the solvent exchange cartridge.

In an embodiment of any one of the foregoing embodiments, the solution that is applied has or is adjusted to have an ascorbic acid concentration of between about 3 mg/mL and about 50 mg/mL, about 3 mg/mL and about 45 mg/mL, about 3 mg/mL and about 40 mg/mL, about 3 mg/mL and about 35 mg/mL, about 3 mg/mL and about 30 mg/mL, about 3 mg/mL and about 25 mg/mL, about 3 mg/mL and about 20 mg/mL, about 3 mg/mL and about 15 mg/mL, about 3 mg/mL and about 10 mg/mL or about 3 mg/mL and about 5 mg/mL. In some embodiments of any one of the methods or compositions provided herein, the ascorbic acid concentration is between about 3.3 and about 7.8 mg/mL. In an embodiment of any one of the foregoing embodiments, the solution that is applied has or is adjusted to have an ascorbic acid concentration of between about 5 mg/mL and about 50 mg/mL, about 10 mg/mL and about 50 mg/mL, about 15 mg/mL and about 50 mg/mL, about 20 mg/mL and about 50 mg/mL, about 25 mg/mL and about 50 mg/mL, about 30 mg/mL and about 50 mg/mL, about 35 mg/mL and about 50 mg/mL, about 40 mg/mL and about 50 mg/mL or about 55 mg/mL and about 50 mg/mL. In an embodiment of any one of the foregoing embodiments, the solution that is applied has or is adjusted to have an ascorbic acid concentration of between about 5 mg/mL and about 45 mg/mL, about 10 mg/mL and about 40 mg/mL, about 15 mg/mL and about 35 mg/mL or about 20 mg/mL and about 30 mg/mL. In an embodiment of any one of the foregoing embodiments, the solution that is applied has or is adjusted to have an ascorbic acid concentration of about 5 mg/mL, about 8 mg/mL, about 10 mg/mL, about 15 mg/mL, about 20 mg/mL, about 25 mg/mL, about 30 mg/mL, about 35 mg/mL, about 40 mg/mL, about 45 mg/mL or about 50 mg/mL. In some embodiments of any one of the methods or compositions provided herein, the ascorbic acid concentration is between about 2.5 and about 7.5, about 3.5 and about 7.5, about 5 and about 7.5, about 2.5 and about 10, about 3.5 and about 10, about 5 and about 10, about 2.5 and about 15, about 3.5 and about 15, or about 5 and about 15 mg/mL. In some embodiments of any one of the methods or compositions provided herein, the ascorbic acid concentration is any one of the values provided herein.

In an embodiment of any one of the foregoing embodiments, the solution that is applied has or is adjusted to have a pH of about 4 or less, about 3.5 or less, about 3 or less, about 2.5 or less, about 2 or less, about 1.5 or less or about 1 or less. In an embodiment of any one of the foregoing embodiments, the solution that is applied has or is adjusted to have a pH of between about 1 and about 4, between about 1.5 and about 4, between about 2 and about 4, between about 2.5 and about 4, between about 3 and about 4 or between about 3.5 and about 4. In an embodiment of any one of the foregoing embodiments, the solution that is applied has or is adjusted to have a pH of between about 1 and about 3.5, between about 1 and about 3, between about 1 and about 2.5, between about 1 and about 2 or between about 1 and about 1.5. In an embodiment of any one of the foregoing embodiments, the solution that is applied has or is adjusted to have a pH of between about 1.5 and about 3.5, between about 1.5 and about 3, between about 1.5 and about 2.5 or between about 1.5 and about 2. In some embodiments of any one of the methods or compositions provided herein, the is any one of the pHs provided herein.

In an embodiment of any one of the foregoing embodiments, the solution further comprises ethanol. In some embodiments, the solution comprises less than or equal to about 30% ethanol (w/v). In some embodiments, the solution comprises less than or equal to about 25% ethanol (w/v). In some embodiments, the solution comprises less than or equal to about 20% ethanol (w/v). In some embodiments, the solution comprises less than or equal to about 15% ethanol (w/v). In some embodiments, the solution comprises less than or equal to about 10% ethanol (w/v). In some embodiments, the solution comprises less than or equal to about 5% ethanol (w/v). In some embodiments, the solution comprises less than or equal to about 3% ethanol (w/v). In one embodiment of any one of the foregoing, the solution comprises at least 3% ethanol (w/v). In some embodiments of any one of the methods or compositions provided herein, the solution comprises ethanol (w/v) at any one of the values provided herein.

In an embodiment of any one of the methods provided herein, the solvent exchange cartridge may be washed after applying the solution comprising [¹⁸F]DCFPyL. In one embodiment of any one of the methods provided herein, [¹⁸F]DCFPyL is substantially retained on the solvent exchange cartridge during the washing. In one embodiment or any one of the methods provided herein, the wash solution is any one of the solutions provided herein with any one of the concentrations of ascorbic acid and any one of the pHs provided herein.

In an embodiment of any one of the methods provided herein, the method further comprises eluting [¹⁸F]DCFPyL from the solvent exchange cartridge with a solvent solution. In one embodiment or any one of the methods provided herein, the solvent solution comprises an alcohol. In some embodiments, the solvent solution comprises any one of the amounts of ethanol as provided herein, such as at least 90% ethanol (w/v). In some embodiments, the solvent solution comprises 100% ethanol (w/v). In some embodiments, the solvent solution comprises less than or equal to about 30% ethanol (w/v). In some embodiments, the solvent solution comprises less than or equal to about 25% ethanol (w/v). In some embodiments, the solvent solution comprises less than or equal to about 20% ethanol (w/v). In some embodiments, the solvent solution comprises less than or equal to about 15% ethanol (w/v). In some embodiments, the solvent solution comprises less than or equal to about 10% ethanol (w/v). In some embodiments, the solvent solution comprises less than or equal to about 5% ethanol (w/v). In some embodiments, the solvent solution comprises less than or equal to about 3% ethanol (w/v). In one embodiment of any one of the foregoing, the solvent solution comprises at least 3% ethanol (w/v). In some embodiments of any one of the methods or compositions provided herein, the solution comprises ethanol (w/v) at any one of the values provided herein.

In an embodiment of any one of the foregoing embodiments, the solvent solution has an ascorbic acid concentration of between about 3 mg/mL and about 50 mg/mL, about 3 mg/mL and about 45 mg/mL, about 3 mg/mL and about 40 mg/mL, about 3 mg/mL and about 35 mg/mL, about 3 mg/mL and about 30 mg/mL, about 3 mg/mL and about 25 mg/mL, about 3 mg/mL and about 20 mg/mL, about 3 mg/mL and about 15 mg/mL, about 3 mg/mL and about 10 mg/mL or about 3 mg/mL and about 5 mg/mL. In some embodiments of any one of the methods or compositions provided herein, the ascorbic acid concentration is between about 3.3 and about 7.8 mg/mL. In an embodiment of any one of the foregoing embodiments, the solvent solution has an ascorbic acid concentration of between about 5 mg/mL and about 50 mg/mL, about 10 mg/mL and about 50 mg/mL, about 15 mg/mL and about 50 mg/mL, about 20 mg/mL and about 50 mg/mL, about 25 mg/mL and about 50 mg/mL, about 30 mg/mL and about 50 mg/mL, about 35 mg/mL and about 50 mg/mL, about 40 mg/mL and about 50 mg/mL or about 55 mg/mL and about 50 mg/mL. In an embodiment of any one of the foregoing embodiments, the solvent solution has an ascorbic acid concentration of between about 5 mg/mL and about 45 mg/mL, about 10 mg/mL and about 40 mg/mL, about 15 mg/mL and about 35 mg/mL or about 20 mg/mL and about 30 mg/mL. In an embodiment of any one of the foregoing embodiments, the solvent solution has an ascorbic acid concentration of about 5 mg/mL, about 8 mg/mL, about 10 mg/mL, about 15 mg/mL, about 20 mg/mL, about 25 mg/mL, about 30 mg/mL, about 35 mg/mL, about 40 mg/mL, about 45 mg/mL or about 50 mg/mL. In some embodiments of any one of the methods or compositions provided herein, the ascorbic acid concentration is between about 2.5 and about 7.5, about 3.5 and about 7.5, about 5 and about 7.5, about 2.5 and about 10, about 3.5 and about 10, about 5 and about 10, about 2.5 and about 15, about 3.5 and about 15, or about 5 and about 15 mg/mL. In some embodiments of any one of the methods or compositions provided herein, the ascorbic acid concentration is any one of the values provided herein. In some embodiments of any one of the methods or compositions provided herein, the ascorbic acid concentration is at any one of the values provided herein.

In an embodiment of any one of the foregoing embodiments, the solvent solution has a pH of about 4 or less, about 3.5 or less, about 3 or less, about 2.5 or less, about 2 or less, about 1.5 or less or about 1 or less. In an embodiment of any one of the foregoing embodiments, the solvent solution has a pH of between about 1 and about 4, between about 1.5 and about 4, between about 2 and about 4, between about 2.5 and about 4, between about 3 and about 4 or between about 3.5 and about 4. In an embodiment of any one of the foregoing embodiments, the solvent solution has a pH of between about 1 and about 3.5, between about 1 and about 3, between about 1 and about 2.5, between about 1 and about 2 or between about 1 and about 1.5. In an embodiment of any one of the foregoing embodiments, the solvent solution has a pH of between about 1.5 and about 3.5, between about 1.5 and about 3, between about 1.5 and about 2.5 or between about 1.5 and about 2. In some embodiments of any one of the methods or compositions provided herein, the pH is any one of the pHs provided herein.

In an embodiment of any one of the methods provided herein, the method further comprises collecting the elution comprising [¹⁸F]DCFPyL in a collection vial, such as a FCV or FPV. In one embodiment of any one of the methods provided herein, the method further comprises maintaining or adjusting the ascorbic acid and pH of the collected solution to any one of the ascorbic acid concentrations and pHs as provided herein.

In some embodiments of any one of the methods provided herein, the pH is maintained or adjusted to be about 2.5, about 3, about 3.5, about 4, about 4.5, about 5, about 5.5, about 6, about 6.5, about 7, about 7.5 or about 8. In some embodiments, the pH is maintained or adjusted to be between about 2.5 and about 7.5. In some embodiments, the pH is maintained or adjusted to be between about 3 and about 7.5. In some embodiments, the pH is maintained or adjusted to be between about 3.5 and about 7.5. In some embodiments, the pH is maintained or adjusted to be between about 4 and about 7.5. In some embodiments, the pH is maintained or adjusted to be between about 4.5 and about 7.5. In some embodiments, the pH is maintained or adjusted to be between about 5 and about 7.5. In some embodiments, the pH is maintained or adjusted to be between about 2.5 and about 7. In some embodiments, the pH is maintained or adjusted to be between about 3 and about 7. In some embodiments, the pH is maintained or adjusted to be between about 3.5 and about 7. In some embodiments, the pH is maintained or adjusted to be between about 4 and about 7. In some embodiments, the pH is maintained or adjusted to be between about 4.5 and about 7. In some embodiments, the pH is maintained or adjusted to be between about 5 and about 7. In some embodiments, the pH is maintained or adjusted to be between about 5.5 and about 7. In some embodiments, the pH is maintained or adjusted to be between about 2.5 and about 6.5. In some embodiments, the pH is maintained or adjusted to be between about 3 and about 6.5. In some embodiments, the pH is maintained or adjusted to be between about 3.5 and about 6.5. In some embodiments, the pH is maintained or adjusted to be between about 4 and about 6.5. In some embodiments, the pH is maintained or adjusted to be between about 4.5 and about 6.5. In some embodiments, the pH is maintained or adjusted to be between about 5 and about 6.5. In some embodiments, the pH is maintained or adjusted to be between about 5.5 and about 6.5. In some embodiments, the pH is maintained or adjusted to be between about 2.5 and about 6. In some embodiments, the pH is maintained or adjusted to be between about 3 and about 6. In some embodiments, the pH is maintained or adjusted to be between about 3.5 and about 6. In some embodiments, the pH is maintained or adjusted to be between about 4 and about 6. In some embodiments, the pH is maintained or adjusted to be between about 4.5 and about 6. In some embodiments, the pH is maintained or adjusted to be between about 5 and about 6. In some embodiments, the pH is maintained or adjusted to be between about 5.5 and about 6. In some embodiments, the pH is maintained or adjusted to be between about 2.5 and about 5.5. In some embodiments, the pH is maintained or adjusted to be between about 3 and about 5.5. In some embodiments, the pH is maintained or adjusted to be between about 3.5 and about 5.5. In some embodiments, the pH is maintained or adjusted to be between about 4 and about 5.5. In some embodiments, the pH is maintained or adjusted to be between about 4.5 and about 5.5. In some embodiments, the pH is maintained or adjusted to be between about 5 and about 5.5. In some embodiments of any one of the methods or compositions provided herein, the pH is any one of the pHs provided herein.

In some embodiments of any one of the methods provided herein, the pH is maintained or adjusted to be about 2.5, about 3.0, about 3.5, about 4.0, about 4.5, about 5.0, about 5.5, about 6.0, about 6.5, about 7.0, about 7.5 or about 8.0. In some embodiments, the pH is maintained or adjusted to be between about 2.5 and about 7.5. In some embodiments, the pH is maintained or adjusted to be between about 3.0 and about 7.5. In some embodiments, the pH is maintained or adjusted to be between about 3.5 and about 7.5. In some embodiments, the pH is maintained or adjusted to be between about 4.0 and about 7.5. In some embodiments, the pH is maintained or adjusted to be between about 4.5 and about 7.5. In some embodiments, the pH is maintained or adjusted to be between about 5.0 and about 7.5. In some embodiments, the pH is maintained or adjusted to be between about 2.5 and about 7.0. In some embodiments, the pH is maintained or adjusted to be between about 3.0 and about 7.0. In some embodiments, the pH is maintained or adjusted to be between about 3.5 and about 7.0. In some embodiments, the pH is maintained or adjusted to be between about 4.0 and about 7.0. In some embodiments, the pH is maintained or adjusted to be between about 4.5 and about 7.0. In some embodiments, the pH is maintained or adjusted to be between about 5.0 and about 7.0. In some embodiments, the pH is maintained or adjusted to be between about 5.5 and about 7.0. In some embodiments, the pH is maintained or adjusted to be between about 2.5 and about 6.5. In some embodiments, the pH is maintained or adjusted to be between about 3.0 and about 6.5. In some embodiments, the pH is maintained or adjusted to be between about 3.5 and about 6.5. In some embodiments, the pH is maintained or adjusted to be between about 4.0 and about 6.5. In some embodiments, the pH is maintained or adjusted to be between about 4.5 and about 6.5. In some embodiments, the pH is maintained or adjusted to be between about 5.0 and about 6.5. In some embodiments, the pH is maintained or adjusted to be between about 5.5 and about 6.5. In some embodiments, the pH is maintained or adjusted to be between about 2.5 and about 6.0. In some embodiments, the pH is maintained or adjusted to be between about 3.0 and about 6.0. In some embodiments, the pH is maintained or adjusted to be between about 3.5 and about 6.0. In some embodiments, the pH is maintained or adjusted to be between about 4.0 and about 6.0. In some embodiments, the pH is maintained or adjusted to be between about 4.5 and about 6.0. In some embodiments, the pH is maintained or adjusted to be between about 5.0 and about 6.0. In some embodiments, the pH is maintained or adjusted to be between about 5.5 and about 6.0. In some embodiments, the pH is maintained or adjusted to be between about 2.5 and about 5.5. In some embodiments, the pH is maintained or adjusted to be between about 3.0 and about 5.5. In some embodiments, the pH is maintained or adjusted to be between about 3.5 and about 5.5. In some embodiments, the pH is maintained or adjusted to be between about 4.0 and about 5.5. In some embodiments, the pH is maintained or adjusted to be between about 4.5 and about 5.5. In some embodiments, the pH is maintained or adjusted to be between about 5.0 and about 5.5. In some embodiments of any one of the methods or compositions provided herein, the pH is any one of the pHs provided herein.

In some embodiments of any one of the methods provided herein, the ascorbic acid is maintained or adjusted to be about 2 mg/mL, about 2.5 mg/mL, about 3.5 mg/mL, about 4 mg/mL, about 4.5 mg/mL, about 5 mg/mL, about 5.5 mg/mL, about 6 mg/mL, about 6.5 mg/mL, about 7 mg/mL, about 7.5 mg/mL, about 8 mg/mL, about 8.5 mg/mL, about 9 mg/mL, about 9.5 mg/mL or about 10 mg/mL. In some embodiments of any one of the methods provided herein, the ascorbic acid is maintained or adjusted to be about 2 mg/mL, about 2.5 mg/mL, about 3.5 mg/mL, about 4.0 mg/mL, about 4.5 mg/mL, about 5.0 mg/mL, about 5.5 mg/mL, about 6.0 mg/mL, about 6.5 mg/mL, about 7.0 mg/mL, about 7.5 mg/mL, about 8.0 mg/mL, about 8.5 mg/mL, about 9.0 mg/mL, about 9.5 mg/mL, about 10 mg/mL or about 15 mg/mL. In some embodiments of any one of the methods or compositions provided herein, the ascorbic acid concentration is maintained or adjusted to be between about 3.3 and about 7.8 mg/mL. In some embodiments of any one of the methods or compositions provided herein, the ascorbic acid concentration is maintained or adjusted to be between about 2.5 and about 7.5, about 3.5 and about 7.5, about 5 and about 7.5, about 2.5 and about 10, about 3.5 and about 10, about 5 and about 10, about 2.5 and about 15, about 3.5 and about 15, or about 5 and about 15 mg/mL. In some embodiments of any one of the methods or compositions provided herein, the ascorbic acid concentration is any one of the values provided herein.

In some embodiments of any one of the methods provided herein, the ascorbic acid concentration is maintained or adjusted to be between about 2.5 and about 7.5 mg/mL. In some embodiments of any one of the methods provided herein, the ascorbic acid concentration is maintained or adjusted to be between about 3 and about 7.5 mg/mL. In some embodiments of any one of the methods provided herein, the ascorbic acid concentration is maintained or adjusted to be between about 3.0 and about 7.5 mg/mL. In some embodiments of any one of the methods provided herein, the ascorbic acid concentration is maintained or adjusted to be between about 3.5 and about 7.5 mg/mL. In some embodiments of any one of the methods provided herein, the ascorbic acid concentration is maintained or adjusted to be between about 4 and about 7.5 mg/mL. In some embodiments of any one of the methods provided herein, the ascorbic acid concentration is maintained or adjusted to be between about 4.0 and about 7.5 mg/mL. In some embodiments of any one of the methods provided herein, the ascorbic acid concentration is maintained or adjusted to be 4.5 and about 7.5 mg/mL. In some embodiments of any one of the methods provided herein, the ascorbic acid concentration is maintained or adjusted to be between about 5 and about 7.5 mg/mL. In some embodiments of any one of the methods provided herein, the ascorbic acid concentration is maintained or adjusted to be between about 5.0 and about 7.5 mg/mL. In some embodiments of any one of the methods provided herein, the ascorbic acid concentration is maintained or adjusted to be between about 2.5 and about 7 mg/mL. In some embodiments of any one of the methods provided herein, the ascorbic acid concentration is maintained or adjusted to be between about 2.5 and about 7.0 mg/mL. In some embodiments of any one of the methods provided herein, the ascorbic acid concentration is maintained or adjusted to be between about 3 and about 7 mg/mL. In some embodiments of any one of the methods provided herein, the ascorbic acid concentration is maintained or adjusted to be between about 3.0 and about 7.0 mg/mL. In some embodiments of any one of the methods provided herein, the ascorbic acid concentration is maintained or adjusted to be between about 3.5 and about 7 mg/mL. In some embodiments of any one of the methods provided herein, the ascorbic acid concentration is maintained or adjusted to be between about 3.5 and about 7.0 mg/mL. In some embodiments of any one of the methods provided herein, the ascorbic acid concentration is maintained or adjusted to be between about 4 and about 7 mg/mL. In some embodiments of any one of the methods provided herein, the ascorbic acid concentration is maintained or adjusted to be between about 4.0 and about 7.0 mg/mL. In some embodiments of any one of the methods provided herein, the ascorbic acid concentration is maintained or adjusted to be between about 4.5 and about 7 mg/mL. In some embodiments of any one of the methods provided herein, the ascorbic acid concentration is maintained or adjusted to be between about 4.5 and about 7.0 mg/mL. In some embodiments of any one of the methods provided herein, the ascorbic acid concentration is maintained or adjusted to be between about 5 and about 7 mg/mL. In some embodiments of any one of the methods provided herein, the ascorbic acid concentration is maintained or adjusted to be between about 5.0 and about 7.0 mg/mL. In some embodiments of any one of the methods provided herein, the ascorbic acid concentration is maintained or adjusted to be between about 5.5 and about 7 mg/mL. In some embodiments of any one of the methods provided herein, the ascorbic acid concentration is maintained or adjusted to be between about 5.5 and about 7.0 mg/mL. In some embodiments of any one of the methods provided herein, the ascorbic acid concentration is maintained or adjusted to be between about 2.5 and about 6.5 mg/mL. In some embodiments of any one of the methods provided herein, the ascorbic acid concentration is maintained or adjusted to be between about 3 and about 6.5 mg/mL. In some embodiments of any one of the methods provided herein, the ascorbic acid concentration is maintained or adjusted to be between about 3.0 and about 6.5 mg/mL. In some embodiments of any one of the methods provided herein, the ascorbic acid concentration is maintained or adjusted to be between about 3.5 and about 6.5 mg/mL. In some embodiments of any one of the methods provided herein, the ascorbic acid concentration is maintained or adjusted to be between about 4 and about 6.5 mg/mL. In some embodiments of any one of the methods provided herein, the ascorbic acid concentration is maintained or adjusted to be between about 4.0 and about 6.5 mg/mL. In some embodiments of any one of the methods provided herein, the ascorbic acid concentration is maintained or adjusted to be between about 4.5 and about 6.5 mg/mL. In some embodiments of any one of the methods provided herein, the ascorbic acid concentration is maintained or adjusted to be between about 5 and about 6.5 mg/mL. In some embodiments of any one of the methods provided herein, the ascorbic acid concentration is maintained or adjusted to be between about 5.0 and about 6.5 mg/mL. In some embodiments of any one of the methods provided herein, the ascorbic acid concentration is maintained or adjusted to be between about 5.5 and about 6.5 mg/mL. In some embodiments of any one of the methods provided herein, the ascorbic acid concentration is maintained or adjusted to be between about 2.5 and about 6 mg/mL. In some embodiments of any one of the methods provided herein, the ascorbic acid concentration is maintained or adjusted to be between about 2.5 and about 6.0 mg/mL. In some embodiments of any one of the methods provided herein, the ascorbic acid concentration is maintained or adjusted to be between about 3 and about 6 mg/mL. In some embodiments of any one of the methods provided herein, the ascorbic acid concentration is maintained or adjusted to be between about 3.0 and about 6.0 mg/mL. In some embodiments of any one of the methods provided herein, the ascorbic acid concentration is maintained or adjusted to be between about 3.5 and about 6 mg/mL. In some embodiments of any one of the methods provided herein, the ascorbic acid concentration is maintained or adjusted to be between about 3.5 and about 6.0 mg/mL. In some embodiments of any one of the methods provided herein, the ascorbic acid concentration is maintained or adjusted to be between about 4 and about 6 mg/mL. In some embodiments of any one of the methods provided herein, the ascorbic acid concentration is maintained or adjusted to be between about 4.0 and about 6.0 mg/mL. In some embodiments of any one of the methods provided herein, the ascorbic acid concentration is maintained or adjusted to be between about 4.5 and about 6 mg/mL. In some embodiments of any one of the methods provided herein, the ascorbic acid concentration is maintained or adjusted to be between about 4.5 and about 6.0 mg/mL. In some embodiments of any one of the methods provided herein, the ascorbic acid concentration is maintained or adjusted to be between about 5 and about 6 mg/mL. In some embodiments of any one of the methods provided herein, the ascorbic acid concentration is maintained or adjusted to be between about 5.0 and about 6.0 mg/mL. In some embodiments of any one of the methods provided herein, the ascorbic acid concentration is maintained or adjusted to be between about 5.5 and about 6 mg/mL. In some embodiments of any one of the methods provided herein, the ascorbic acid concentration is maintained or adjusted to be between about 5.5 and about 6.0 mg/mL. In some embodiments of any one of the methods provided herein, the ascorbic acid concentration is maintained or adjusted to be between about 2.5 and about 5.5 mg/mL. In some embodiments of any one of the methods provided herein, the ascorbic acid concentration is maintained or adjusted to be between about 3 and about 5.5 mg/mL. In some embodiments of any one of the methods provided herein, the ascorbic acid concentration is maintained or adjusted to be between about 3.0 and about 5.5 mg/mL. In some embodiments of any one of the methods provided herein, the ascorbic acid concentration is maintained or adjusted to be between about 3.5 and about 5.5 mg/mL. In some embodiments of any one of the methods provided herein, the ascorbic acid concentration is maintained or adjusted to be between about 4 and about 5.5 mg/mL. In some embodiments of any one of the methods provided herein, the ascorbic acid concentration is maintained or adjusted to be between about 4.0 and about 5.5 mg/mL. In some embodiments of any one of the methods provided herein, the ascorbic acid concentration is maintained or adjusted to be between about 4.5 and about 5.5 mg/mL. In some embodiments of any one of the methods provided herein, the ascorbic acid concentration is maintained or adjusted to be between about 5 and about 5.5 mg/mL. In some embodiments of any one of the methods provided herein, the ascorbic acid concentration is maintained or adjusted to be between about 5.0 and about 5.5 mg/mL. In some embodiments of any one of the methods or compositions provided herein, the ascorbic acid concentration is at any one of the values provided herein.

Without wishing to be bound by theory, the exemplary methods provided herein may also be useful to remove impurities from a composition comprising [¹⁸F]DCFPyL and/or to exchange the solvent in which [¹⁸F]DCFPyL is present, thus allowing for formation of an imaging or diagnostic composition. For example, a solution may be obtained, such as from a purification of [¹⁸F]DCFPyL (e.g., via HPLC or another purification method)), and may comprise impurities and/or solvents which are not suitable for administration to a subject. Accordingly, the impurities may be removed and/or the solvents may be exchanged using a method provided herein.

As a further example, the solution of any one of the methods or compositions provided herein may comprise ascorbic acid, [¹⁸F]DCFPyL, and one or more solvents and/or impurities. In an embodiment of any one of the methods provided herein, the solution may be applied to a solvent exchange cartridge, wherein [¹⁸F]DCFPyL is substantially retained and the other components (e.g., solvents such as acetonitrile and/or impurities) may be removed via elution (e.g., in a step of washing the resin). In an embodiment of any one of the methods provided herein, [¹⁸F]DCFPyL may be recovered by eluting with a solvent solution. In an embodiment of any one of the methods provided herein, the resulting solvent solution containing [¹⁸F]DCFPyL may then be further diluted, if desired, to form an imaging or a diagnostic composition suitable for administration to a subject.

In another example, the solution of any one of the methods or compositions provided herein may comprise acetonitrile (or another solvent, for example, which is not suitable for administration to a subject). In an embodiment of any one of the methods provided herein, the acetonitrile (and/or impurities) may not adhere to the solvent exchange cartridge and may thus be eluted or washed through. Accordingly, the solutions formed by eluting [¹⁸F]DCFPyL from the resin in an embodiment of any one of the methods provided herein may not substantially comprise the acetonitrile (or other solvent). In some embodiments of any one of the methods or compositions provided herein, a first solution may be a composition according to any one aspect or embodiment of the invention described herein.

Accordingly, in embodiments of any one of the methods or compositions provided herein, the solution comprises acetonitrile at a level that is no more than about 0.05% (w/v), no more than about 0.04% (w/v), no more than about 0.03% (w/v), no more than about 0.02% (w/v) or no more than about 0.01% (w/v). In some embodiments of any one of the methods or compositions provided herein, the solution comprises acetonitrile (w/v) at any one of the values provided herein.

The eluting solvent of any one of the methods or compositions provided herein may be any solvent which allows for elution of [¹⁸F]DCFPyL. Generally, [¹⁸F]DCFPyL is substantially soluble in the eluting solvent. In some embodiments of any one of the methods or compositions provided herein, where the solvent in the eluting solution is an alcohol, such as ethanol, the alcohol may be the alcohol contained in the final imaging or diagnostic composition. The ethanol of such embodiments may be at a concentration of any one of the embodiments provided herein. Suitable cartridges and the like are known to those of ordinary skill in the art including HLB and SEP-PAK.

### Radiochemical Purity, Stability, and Radioactive Concentration

The compositions described herein and/or prepared according to the methods described herein may have a high radiochemical purity and/or may maintain the high radiochemical purity for a substantial period of time. As used herein, radiochemical purity refers to the proportion of the amount of radioactivity (from a given radioisotope) present in a specific radiopharmaceutical relative to the total amount of radioactivity (from the same radioisotope) in a composition that comprises the specific radiopharmaceutical. Radiochemical purity can be a measure of the degree of degradation and/or decomposition and/or conversion of the specific radiopharmaceutical into other compounds that may or may not comprise the radioisotope. Radiochemical purity can be the distribution of radioactivity in the parent species versus other radioactive species.

In some embodiments of any one of the methods or compositions provided herein, a composition has a radiochemical purity of at least about 90%. In some embodiments of any one of the methods or compositions provided herein, a composition has a radiochemical purity of at least about 95%. In some embodiments of any one of the methods or compositions provided herein, a composition has a radiochemical purity of at least about 96%. In some embodiments of any one of the methods or compositions provided herein, a composition has a radiochemical purity of at least about 97%. In some embodiments of any one of the methods or compositions provided herein, a composition has a radiochemical purity of at least about 98%. In some embodiments of any one of the methods or compositions provided herein, a composition has a radiochemical purity of at least about 98.5%. In some embodiments of any one of the methods or compositions provided herein, a composition has a radiochemical purity of at least about 98.9%. In some embodiments of any one of the methods or compositions provided herein, a composition has a radiochemical purity of at least about 99%. In some embodiments of any one of the methods or compositions provided herein, a composition has a radiochemical purity of at least about 99.5%. In some embodiments of any one of the methods or compositions provided herein, a composition has a radiochemical purity of at least about 99.9%. In some embodiments of any one of the methods or compositions provided herein, a composition has a radiochemical purity between about 95% and about 98%. In some embodiments of any one of the methods or compositions provided herein, a composition has a radiochemical purity between about 95% and about 98.5%. In some embodiments of any one of the methods or compositions provided herein, a composition has a radiochemical purity between about 95% and about 98.9%. In some embodiments of any one of the methods or compositions provided herein, a composition has a radiochemical purity between about 95% and about 99%. In some embodiments of any one of the methods or compositions provided herein, a composition has a radiochemical purity between about 95% and about 99.5%. In some embodiments of any one of the methods or compositions provided herein, a composition has a radiochemical purity between about 95% and about 99.9%. In some embodiments of any one of the methods or compositions provided herein, a composition has a radiochemical purity between about 95% and about 100%. In some embodiments of any one of the methods or compositions provided herein, a composition has a radiochemical purity of any one of the values provided herein.

Those of ordinary skill in the art will be aware of techniques and systems for determining the radiochemical purity of a composition. In some cases, the radiochemical purity is determined using an HPLC associated with a radio-detector. Generally, the radiochemical purity is determined under ambient conditions (e.g., ambient temperature, ambient humidity, ambient light, etc.), such as in samples stored under such conditions.

In some embodiments of any one of the methods or compositions provided herein, a composition maintains a high radiochemical purity for a substantial period of time. Without wishing to be bound by theory, this may be due to the selection of appropriate composition components and conditions which aid in the stability of the imaging agent. For example, the presence of ascorbic acid and/or ethanol and/or selection of an appropriate composition pH can greatly affect the radiostability of the imaging agent.

In some embodiments of any one of the methods or compositions provided herein, a composition has a radiochemical purity as provided herein at the end of synthesis (EOS). In some embodiments of any one of the methods or compositions provided herein, a composition has a radiochemical purity of at least about 90% over a period of at least about 6 hours, at least 8 hours or at least 10 hours. In some embodiments of any one of the methods or compositions provided herein, a composition has a radiochemical purity of at least about 95% at about 10 hours. In some embodiments of any one of the methods or compositions provided herein, a composition has a radiochemical purity of at least about 97% at about 10 hours. In some embodiments of any one of the methods or compositions provided herein, a composition has a radiochemical purity of at least 99% for at least 10 hours. In such embodiments, the time is measured post synthesis (i.e., the time beyond end of synthesis (EOS)).

In some embodiments of any one of the methods or compositions provided herein, the compositions provided herein exhibit little to no radiolysis, such as at least about 6, 7, 8, 9, or 10 hours post synthesis. The compositions provided herein in any one embodiment has less than about 5% free ¹⁸F, less than about 4.5% free ¹⁸F, less than about 4% free ¹⁸F, less than about 3.5% free ¹⁸F, less than about 3% free ¹⁸F, less than about 2.5% free ¹⁸F, less than about 2% free ¹⁸F, less than about 1.5% free ¹⁸F, less than about 1% free ¹⁸F or less than about 0.5% free ¹⁸F, such as at least about 6, 7, 8, 9, or 10 hours post synthesis. In some embodiments of any one of the methods or compositions provided herein, a composition has any one of the levels of free ¹⁸F provided herein.

In one embodiment of any one of the compositions or methods provided herein, the incorporation of ascorbate in the collection vial prevents radiolysis during transfer and attachment to the solvent exchange cartridge. This can lead to reduced impurities in the drug product at T0. In one embodiment of any one of the compositions or methods provided herein, the presence of ascorbate in the FCV or FPV can stabilize the product during storage for 10 hrs.

In one aspect, a method of detecting free ¹⁸F in a sample is provided herein. In any one of such methods TEAF is used, such as at any one of the concentrations provided herein. In any one of such methods, the composition may be a composition comprising a radiopharmaceutical, such as [¹⁸F]DCFPyL. Such methods comprise adding tetraethylammonium fluoride to a composition to be assessed; and performing an assay on the sample to determine the level of free ¹⁸F, such as with chromatography, such as HPLC, on the composition.

In some embodiments of any one of the methods or compositions provided herein, the radioactive concentration of the composition is at least about 80 mCi/mL, at least about 85 mCi/mL, at least about 90 mCi/mL, at least about 95 mCi/mL, at least about 100 mCi/mL, at least about 105 mCi/mL, at least about 110 mCi/mL, at least about 115 mCi/mL, at least about 120 mCi/mL, at least about 125 mCi/mL, at least about 130 mCi/mL, at least about 135 mCi/mL, at least about 140 mCi/mL, at least about 145 mCi/mL, at least about 150 mCi/mL, at least about 155 mCi/mL, at least about 160 mCi/mL, at least about 165 mCi/mL, at least about 166 mCi/mL, at least about 167 mCi/mL, at least about 168 mCi/mL, at least about 169 mCi/mL or at least about 170 mCi/mL. In some embodiments of any one of the methods or compositions provided herein, the radioactive concentration of the composition is any one of the values provided herein.

In some embodiments of any one of the methods or compositions provided herein, the radioactive concentration of the composition is between about 80 mCi/mL and about 170 mCi/mL, between about 80 mCi/mL and about 165 mCi/mL, between about 80 mCi/mL and about 160 mCi/mL, between about 80 mCi/mL and about 155 mCi/mL, between about 80 mCi/mL and about 150 mCi/mL, between about 80 mCi/mL and about 145 mCi/mL, between about 80 mCi/mL and about 140 mCi/mL, between about 80 mCi/mL and about 135 mCi/mL, between about 80 mCi/mL and about 130 mCi/mL, between about 80 mCi/mL and about 125 mCi/mL, or between about 80 mCi/mL and about 120 mCi/mL. In some embodiments of any one of the methods or compositions provided herein, the radioactive concentration of the composition is between about 85 mCi/mL and about 170 mCi/mL, between about 90 mCi/mL and about 170 mCi/mL, between about 95 mCi/mL and about 170 mCi/mL, between about 100 mCi/mL and about 170 mCi/mL, between about 105 mCi/mL and about 170 mCi/mL, between about 110 mCi/mL and about 170 mCi/mL, between about 115 mCi/mL and about 170 mCi/mL, between about 120 mCi/mL and about 170 mCi/mL, between about 125 mCi/mL and about 170 mCi/mL, between about 130 mCi/mL and about 170 mCi/mL, between about 135 mCi/mL and about 170 mCi/mL, between about 140 mCi/mL and about 170 mCi/mL, between about 145 mCi/mL and about 170 mCi/mL, between about 150 mCi/mL and about 170 mCi/mL, between about 155 mCi/mL and about 170 mCi/mL, between about 160 mCi/mL and about 170 mCi/mL or between about 165 mCi/mL and about 170 mCi/mL. In some embodiments of any one of the methods or compositions provided herein, the radioactive concentration of the composition is between about 85 mCi/mL and about 165 mCi/mL, between about 90 mCi/mL and about 165 mCi/mL, between about 95 mCi/mL and about 165 mCi/mL, between about 100 mCi/mL and about 165 mCi/mL, between about 105 mCi/mL and about 165 mCi/mL, between about 110 mCi/mL and about 165 mCi/mL, between about 115 mCi/mL and about 165 mCi/mL, between about 120 mCi/mL and about 165 mCi/mL, between about 125 mCi/mL and about 165 mCi/mL, between about 130 mCi/mL and about 165 mCi/mL, between about 135 mCi/mL and about 165 mCi/mL, between about 140 mCi/mL and about 165 mCi/mL, between about 145 mCi/mL and about 165 mCi/mL, between about 150 mCi/mL and about 165 mCi/mL, between about 155 mCi/mL and about 165 mCi/mL or between about 160 mCi/mL and about 165 mCi/mL.

In some embodiments of any one of the methods or compositions provided herein, the radioactive concentration of the composition is equal to or less than or equal to about 170 mCi/mL. In some embodiments of any one of the methods or compositions provided herein, the radioactive concentration of the composition is equal to or less than about 169 mCi/mL, equal to or less than about 168 mCi/mL, equal to or less than about 167 mCi/mL, equal to or less than about 166 mCi/mL or equal to or less than about 165 mCi/mL.

As an example, a composition comprising [¹⁸F]DCFPyL and one or more or all of the below features is provided. In an embodiment, the composition has ≥95% [¹⁸F]DCFPyL (radiochemical purity), a pH of 4.5 to 7.0, and 5 to 15 mg/mL ascorbic acid. In an embodiment of any one of the foregoing compositions, the composition has a radioactivity concentration of 1 to 125 mCi/mL at end-of-synthesis (EOS) (e.g., at least 80 mCi/mL, at least 85 mCi/mL, at least 90 mCi/mL, at least 95 mCi/mL, at least 100 mCi/mL, at least 105 mCi/mL, at least 110 mCi/mL, at least 115 mCi/mL, at least 120 mCi/mL, at least 125 mCi/mL, at least 80 mCi/mL to 125 mCi/mL, at least 85 mCi/mL to 125 mCi/mL, at least 90 mCi/mL to 125 mCi/mL, at least 95 mCi/mL to 125 mCi/mL, at least 100 mCi/mL to 125 mCi/mL, at least 105 mCi/mL to 125 mCi/mL, at least 110 mCi/mL to 125 mCi/mL, at least 115 mCi/mL to 125 mCi/mL, or at least 120 mCi/mL to 125 mCi/mL) and/or a specific activity (referenced to EOS)≥1000 mCi/μmol at expiration time. In an embodiment of any one of the foregoing compositions, the composition comprises ethanol≤7.89% w/v and/or acetonitrile≤0.04% w/v. In an embodiment of any one of the foregoing compositions, the composition comprises a total of unknown impurities of ≤1.5 μg/mL and/or an ascorbic acid-related impurity of ≤5.0 μg/mL, such as at relative retention time (RRT) 0.607. In an embodiment of any one of the foregoing compositions, the radioactive identity calculation is within ±10% and/or radionuclidic identity by half-life (T_{1/2}) is 105 to 115 min. The foregoing features as well as the features of the below table may be assayed, measured or assessed with methods known to those of ordinary skill in the art or as provided in the below table, respectively.

TABLE 32
TEST PLAN/SPECIFICATIONS: RELEASE

| | | | |
|---|---|---|---|
| Test | Method | Qty | Acceptance Criteria |

Testing: Physical

| | | | |
|---|---|---|---|
| Appearance | Visual, USP | 1 vial | Clear, colorless to pale yellow, particulate- |
| | | | free solution |
| Membrane Filter | Bubble point | 1 filter | Bubble point ≥ 50 psi |
| Integrity | pressure test | | |

Testing: Chemical

| | | | |
|---|---|---|---|
| Chemical Purity | HPLC/UV | 1 vial¹ | Total of unknown impurities ≤ 1.5 μg/mL |
| pH | pH test with | 1 vial¹ | 4.5 to 7.0 |
| | suitable pH paper | | |
| | or pH meter, USP | | |
| Ascorbic Acid | Color test with AA | 1 vial¹ | 5 to 15 mg/ml |
| | paper strip | | |
| Specified | HPLC/UV | 1 vial¹ | Ascorbic Acid related impurity |
| Unidentified | | | at RRT 0.607 ≤ 5.0 μg/mL |
| Ascorbic Acid | | | |
| Related Impurity | | | |
| Residual Solvents | GC-FID | 1 vial¹ | Ethanol ≤ 7.89% w/v |
| Analysis | | | Acetonitrile ≤ 0.04% w/v |

Testing: Radiochemical

| | | | |
|---|---|---|---|
| Radiochemical | Radiometric | 1 vial¹ | ¹⁸F-DCFPyL ≥ 95% |
| Purity | HPLC | | |
| Radiochemical | Radiometric | 1 vial¹ | Calculation within ±10% |
| Identity* | HPLC | | |
| Radionuclidic | USP | 1 vial¹ | T_{1/2} = 105 to 115 min |
| Identity by Half-life | | | |
| Radioactivity | Dose calibrator | 1 vial¹ | 1 to 125 mCi/mL at EOS |
| Concentration | | | |
| Specific Activity | Calculation from | N/A | ≥1000 mCi/μmol at expiration time |
| (referenced to end- | radioactivity | | |
| of-synthesis) | measurement | | |
| | and chemical | | |
| | concentration by | | |
| | HPLC with UV | | |
| | detection | | |

Testing: Microbiological

| | | | |
|---|---|---|---|
| Endotoxin | Charles River, | 1 cartridge | <11 EU/mL** |
| | USP | | |
| Sterility*** | USP; test must | 2 tubes | Sterile. No growth observed |
| | be started within | | |
| | 30 hours from | | |
| | EOS | | |

¹Testing may be performed on aliquots taken from the same vial.
${}^{*} Radiochemical Identity = \frac{\begin{matrix}Largest RAD peak RT - Avg [ 19 F ] DCFPyL RTin \\ std * 100 \%\end{matrix}}{Avg [ 19 F ] DCFPyLpeak RT in std}$ **Based on a maximum unit dose volume of 10 mL
***Tested post-release

### Imaging Agents and Related Methods

Imaging agents allow for the detection, imaging, and/or monitoring of the presence and/or progression of a condition, pathological disorder, and/or disease. Typically, an imaging agent is administered to a subject in order to provide information relating to at least a portion of the subject (e.g., a human). In some cases, an imaging agent may be used to highlight a specific area of a subject, rendering organs, blood vessels, tissues, and/or other portions more detectable and/or more clearly imaged. By increasing the detectability and/or image quality of the object being studied, the presence and extent of disease and/or condition can be determined.

The imaging agent as provided herein is [¹⁸F]DCFPyL, where the radioisotope, ¹⁸F, is the imaging moiety, particularly positron emission tomography (PET) imaging in an embodiment. The compositions provided herein may be used in nuclear medicine imaging. In some embodiments, the compositions provided may be pharmaceutically acceptable. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

[¹⁸F]DCFPyL may also be present as pharmaceutically acceptable salts in the compositions provided herein. The pharmaceutically acceptable salt may be a derivative of a disclosed compound wherein the parent compound is modified by making acid or base salts thereof. Examples of pharmaceutically acceptable salts include, but are not limited to, mineral or organic acid salts of basic residues such as amines; and alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, and nitric; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, and isethionic.

In some embodiments, [¹⁸F]DCFPyL may be synthesized using an automated synthesis module. Automated synthesis modules will be known to those of ordinary skill in the art. In some cases, an imaging agent may be synthesized according to the teachings of automated synthesis modules described in International Patent Publication No. WO2011/097649, published Aug. 11, 2011, the teachings of which relating to automated synthesis modules being incorporated by reference herein.

In some embodiments, the imaging or diagnostic compositions described herein may find application in methods of imaging, including methods of imaging a subject that includes administering an imaging or diagnostic composition as described herein, and imaging a region of the subject that is of interest. Regions of interest may include, but are not limited to, cancerous tissues, tissues and organs with tumors and/or metastases, the prostate, and blood vessels (e.g., arteries, veins). Also provided herein are methods of imaging one or more cells, organs or tissues comprising contacting the cells, organs or tissues or administering to a subject an effective amount of a compound as provided herein. In some embodiments, the one or more organs or tissues include prostate tissue, kidney tissue, brain tissue, vascular tissue or tumor tissue. In embodiments, the subject is a human subject.

In some embodiments, the imaging method is suitable for imaging by targeting PSMA. In some embodiments, the imaging method is suitable for imaging of cancer, tumor or neoplasm. In some embodiments, the cancer is selected from eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain cancer (e.g., gliomas), throat cancer, skin melanoma, acute lymphocytic leukemia, acute myelogenous leukemia, Ewing's Sarcoma, Kaposi's Sarcoma, basal cell carcinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

The imaging methods provided herein are suitable for imaging any physiological process or feature in which PSMA is involved. In some embodiments, the imaging methods are suitable for identification of areas of tissues or targets which express high concentrations of PSMA. Exemplary applications include imaging glutamateric neurotransmission, presynaptic glutamatergic neurotransmission, malignant tumors or cancers that express PSMA, prostate cancer (including metastasized prostate cancer), and angiogenesis. Solid tumors express PSMA in the neovasculature. Therefore, methods and compositions provided herein can be used to image solid tumors including lung, renal cell, glioblastoma, pancreas, bladder, sarcoma, melanoma, breast, colon, germ cell, pheochromocytoma, esophageal and stomach. PSMA is frequently expressed in endothelial cells of capillary vessels in peritumoral and endotumoral areas of various malignancies such that the methods and compositions provided can be used for imaging such malignancies. Also, certain benign lesions and tissues including endometrium, schwannoma and Barrett's esophagus can be imaged according to the methods and compositions provided.

The methods and compositions for imaging angiogenesis provided are suitable for use in imaging a variety of diseases and disorders in which angiogenesis takes place. Illustrative, non-limiting, examples include tumors, collagen vascular disease, cancer, stroke, vascular malformations, and retinopathy. Methods and compositions of imaging angiogenesis provided are also suitable for use in diagnosis and observation of normal tissue development.

In some embodiments, any one of the methods provided herein may include diagnosing or assisting in diagnosing a disease or condition, assessing and/or selecting and/or changing treatment of a disease or condition, assessing efficacy of treatment of a disease or condition, or imaging in a subject with a known or suspected disease or condition.

In some embodiments of any one of the methods provided herein, a method of imaging includes (a) administering to a subject an imaging or diagnostic composition that includes an imaging agent, and (b) acquiring at least one image of at least a portion of the subject. In some embodiments, any one of the methods provided herein acquiring employs positron emission tomography (PET) for visualizing the distribution of the imaging agent within at least a portion of the subject. As will be understood by those of ordinary skill in the art, imaging may include full body imaging of a subject, or imaging of a specific body region or tissue of the subject that is of interest. For example, if a subject is known to have, or is suspected of having cancer, such as prostate cancer, methods may be used to image organs with a primary tumor, such as the prostate, and/or organs with (or suspected to have) metastases of the subject.

In one embodiment of any one of the compositions or methods provided herein, the amount of PyL is 8-10 mCi. In one embodiment of any one of the compositions or methods provided herein, the amount of PyL administered to a patient is 8-10 or 8, 9 or 10 mCi from a FPV prepared at any one of the concentrations provided herein, such as 80-170 mCi/mL, and containing ascorbate at any one of the concentrations provided herein, such as 5.6 mg/mL ascorbate. In one embodiment of any one of the compositions or methods provided herein, the amount of PyL administered to a patient is 8-10 or 8, 9 or 10 mCi from a FPV prepared at any one of the concentrations provided herein, such as 80-125, 80-130, 80-135, 80-140, 80-145, 80-150, 80-155, 80-160, or 80-165 mCi/mL, and containing ascorbate at any one of the concentrations provided herein, such as 5.6 mg/mL ascorbate, with a pH of any one of the pHs provided herein, such as 4.5 to 7 or 7.0 and with any one of the radiochemical purities provided herein, such as at least 90% (such as up to 10 hours following EOS), and/or any one of the specific activities provided herein, such as of at least 1000 mCi/μmol, at the time of administration. In an embodiment of any one of the foregoing the composition comprises ≤78.9 mg ethanol in 0.9% sodium chloride (injection USP). In one embodiment of any one of the foregoing, imaging with a PET camera occurs 1 hr after administration.

In some embodiments of any one of the methods or compositions provided herein, the radiolabeled compound is detected by positron emission tomography (PET) or position emission tomography/computed tomography (PET/CT). Images can be generated by virtue of differences in the spatial distribution of the imaging agents which accumulate at a site. The spatial distribution may be measured using any means suitable for the particular label, for example, a gamma camera, a PET apparatus, a PET/CT apparatus, and the like. The extent of accumulation of the imaging agent may be quantified using known methods for quantifying radioactive emissions.

In general, a detectably effective amount of a composition provided herein for imaging can be administered to a subject. In accordance with the invention, “a detectably effective amount” is defined as an amount sufficient to yield an acceptable image using equipment which is available for clinical use. A detectably effective amount of a composition provided herein may be administered in one or more injections. The detectably effective amount can vary according to factors such as the degree of susceptibility of the individual, the age, sex, and weight of the individual, idiosyncratic responses of the individual, and the dosimetry. Detectably effective amounts can also vary according to instrument and film-related factors. Optimization of such factors is well within the level of skill in the art. The amount of an imaging agent used for diagnostic purposes and the duration of the imaging study will depend upon the imaging agent, the body mass of the patient, the nature and severity of the condition being treated, the nature of therapeutic treatments which the patient has under-gone, and on the idiosyncratic responses of the patient. Ultimately, the attending physician can decide the amount to administer to each individual patient and the duration of the imaging study.

In one embodiment of any one of the methods or compositions provided herein, the subject is a human, rat, mouse, cat, dog, horse, sheep, cow, monkey, avian, or amphibian. In another embodiment of any one of the methods or compositions provided herein, the cell is in vivo or in vitro. Typical subjects to which compounds of the invention may be administered are mammals, such as primates and humans. For veterinary applications, a wide variety of subjects include, e.g., livestock such as cattle, sheep, goats, cows, swine and the like; poultry such as chickens, ducks, geese, turkeys, and the like; and domesticated animals particularly pets such as dogs and cats. For diagnostic or research applications, a wide variety of mammals are suitable subjects including rodents (e.g., mice, rats, hamsters), rabbits, primates, and swine such as inbred pigs and the like. Additionally, for in vitro applications, such as in vitro diagnostic and research applications, body fluids and cell samples of any of the above subjects are suitable for use, such as human, blood, urine or tissue samples.

### Kits

Also provided are kits comprising any one of the compositions, reagents or combinations of reagents provided herein. In some embodiments, the kit provides packaged pharmaceutical compositions comprising a pharmaceutically acceptable carrier and a composition of the invention. In other embodiments, the kit provides the compounds and reagents necessary to practice any one of the methods provided herein. Thus, in some embodiments, a kit as provided herein comprises the combination of reagents of any one of the methods provided herein. In one embodiment of any one of the kits provided, the kit further comprises indicia comprising at least one of: instructions for performing any one of the methods provided herein, for preparing any one of the compositions provided herein, and/or for using a final composition as provided herein in a method of use, such as any one of the methods of use provided herein. In one embodiment of any one of the kits provided, the kit comprises any one of the compositions provided herein in combination with a pharmaceutically acceptable carrier. The composition of any one of the kits provided may be in solution. In one embodiment of any one of the kits provided, the final composition is provided in a syringe or vial or the kit further comprises a syringe or vial, such as one useful for administration, such as a FCV or FPV.

In one embodiment of any one of the kits provided herein, the kit is for synthesizing PyL. In one embodiment of any one of the kits provided herein, the kit comprises ascorbic acid, such as at any one of the concentrations provided herein, or ascorbate in an amount sufficient to prepare ascorbic acid at any one of the concentrations provided herein. In one embodiment of the foregoing, the kit further comprises PyL precursor, saline and/or phosphoric acid. In one embodiment of the foregoing, the kit further comprises one or more collection vials and/or one or more FCVs and/or FPVs. In one embodiment of any one of the kits provided herein, the kit comprises a cassette for automated synthesis, such as All-in-One synthesis. In one embodiment of any one of the kits provided, the automated synthesis is for synthesizing PyL for PET imaging.

### EXAMPLES

In order that the disclosure described herein may be more fully understood, the following examples are set forth. The synthetic and biological examples described in this application are offered to illustrate the compounds, pharmaceutical compositions, and methods provided herein and are not to be construed in any way as limiting their scope.

### Example 1. Synthesis of [¹⁸F]DCFPyL

The 5-(((S)-6-(tert-butoxy)-5-(3-((S)-1,5-di-tert-butoxy-1,5-dioxopentan-2-yl)ureido)-6-oxohexyl)carbamoyl)-N,N,N-trimethylpyridin-2-aminium trifluoromethanesulfonate precursor was custom synthesized as was the ((2S)-2-[[(1S)-1-carboxy-5-[(6-fluoranylpyridine-3-carbonyl)-amino]-pentyl]-carbamoyl-amino]pentanedioic acid reference standard. Synthesis reagents were from a FDA-approved Trasis kit. Ascorbic acid and citric acid were USP. The synthesis of 2-(3-{1-carboxy-5-[(6-[18F]fluoro-pyridine-3-carbonyl)-amino]-pentyl}ureido)-pentanedioic acid was performed on an All-in-One module (Trasis). Cyclotron produced ¹⁸F was captured.

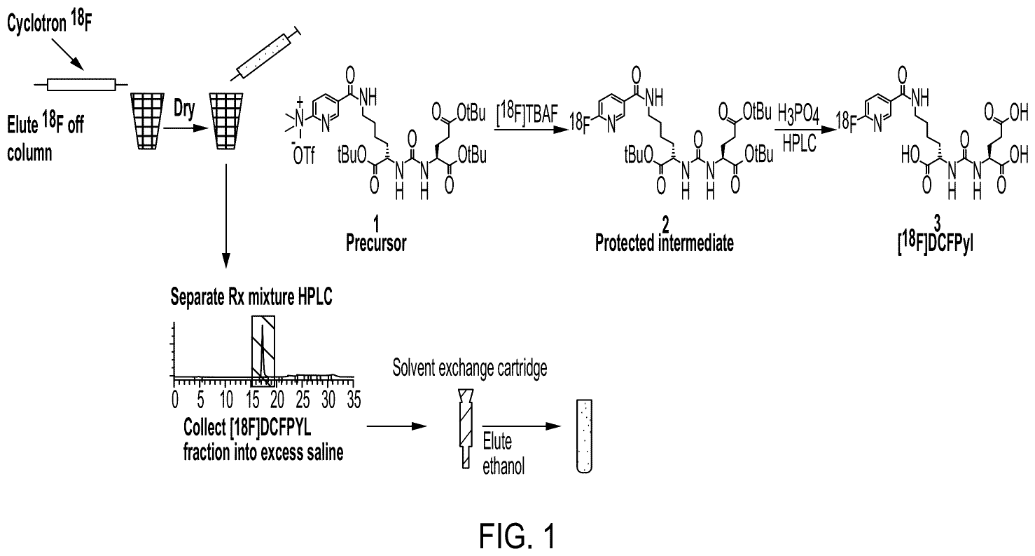

All chemicals and components were loaded onto the ALL-in-One synthesis cassette with cyclotron produced [¹⁸F]fluoride ion captured on a SPE cartridge. The resin cartridge was eluted into the reaction vessel and dried. The reaction vessel was cooled and a solution of the [¹⁸F]DCFPyL precursor was added to the reaction vessel containing the dried [¹⁸F]fluoride, and the solution was heated. After the labeling reaction was complete phosphoric acid (85%) was added to the reaction to facilitate removal of the tert-butoxy protecting groups. Sodium hydroxide was then added to quench the de-protection reaction. The [¹⁸F]DCFPyL was purified from this reaction mixture by semi-preparative HPLC. The [¹⁸F]DCFPyL product peak was collected based on radio-detection and then the solvent was exchanged by retention and elution off a separation cartridge. Elution was in ethanol, and this was then diluted into saline alone or saline containing additional radioprotector (FIG. 1).

Stability was assessed using reverse phase HPLC on a C18 column with a phosphate buffer/acetonitrile gradient mobile phase, and the elution profile was monitored using both UV and radiochemical detectors. Samples were analyzed either by direct injection onto the HPLC, dilution and injection or including the addition of tetraethylammonium fluoride (TEAF) into the sample before injection.

The solution pH was determined either by spotting on a pH paper strip or using a pH probe and meter. Ascorbic acid concentration was verified by paper spotting on an ascorbic acid detection paper strip.

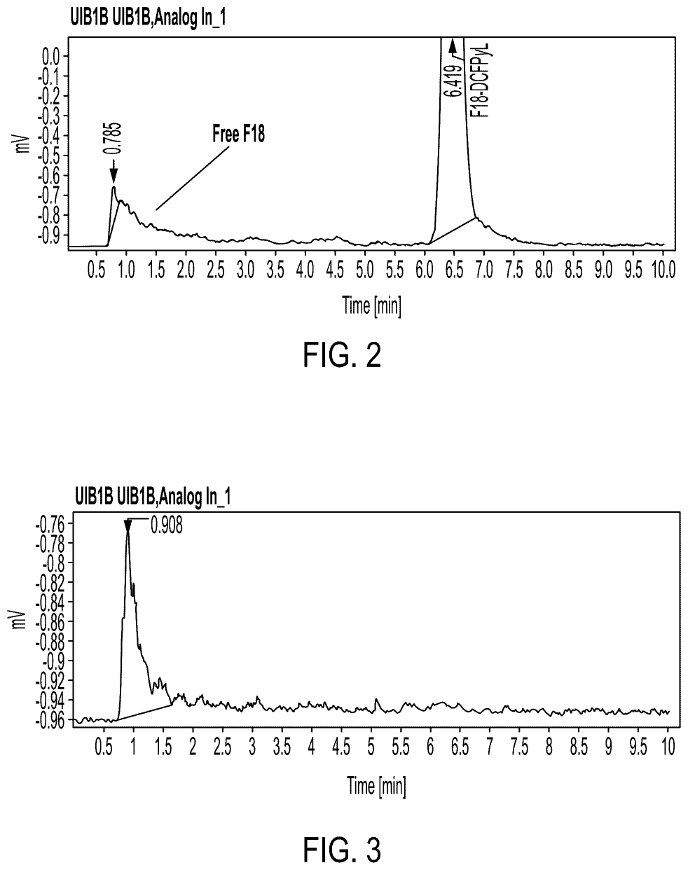

### Example 2. Radiolysis Assessment Method

[¹⁸F]DCFPyL is generally radiolytically stable with the exception that an early running peak on the analytic method was noted during Process Validation (PV) lots. This early running peak exhibited extensive tailing and co-eluted with free ¹⁸F. The bad peak shape resulted in poor integration of the free ¹⁸F peak in the RCP chromatogram and the potential for underestimation of the impurity (FIG. 2).

A method for improving HPLC radiodetector quantification of free ¹⁸F incorporated potassium fluoride (15 mg/mL) in the analytic sample to sharpen the free ¹⁸F peak (Journal of Pharmaceutical and Biomedical Analysis 111 2015 209-214) can be used. However, studies with tetraethylammonium fluoride (TEAF, 44 mg/mL) (FIG. 3) demonstrated improved peak shape. Potassium fluoride resulted in worsening of the [¹⁸F]DCFPyL peak shape, and TEAF was selected.

Known amounts of free ¹⁸F either alone or spiked with TEAF were injected onto the analytic column and the eluent from the column collected over the course of a chromatogram and assaying the radioactivity using a Capintec. The results showed 90.9% and 101.3% recoveries for free ¹⁸F alone and free ¹⁸F spiked with TEAF, respectively. This demonstrates an improvement in free ¹⁸F recovery when samples are spiked with TEAF in addition to the peak shape improvement described above. Adding TEAF into the analytic samples was incorporated into subsequent studies examining potential radiolysis of [¹⁸F]DCFPyL to overcome some of the limitations of free ¹⁸F quantification.

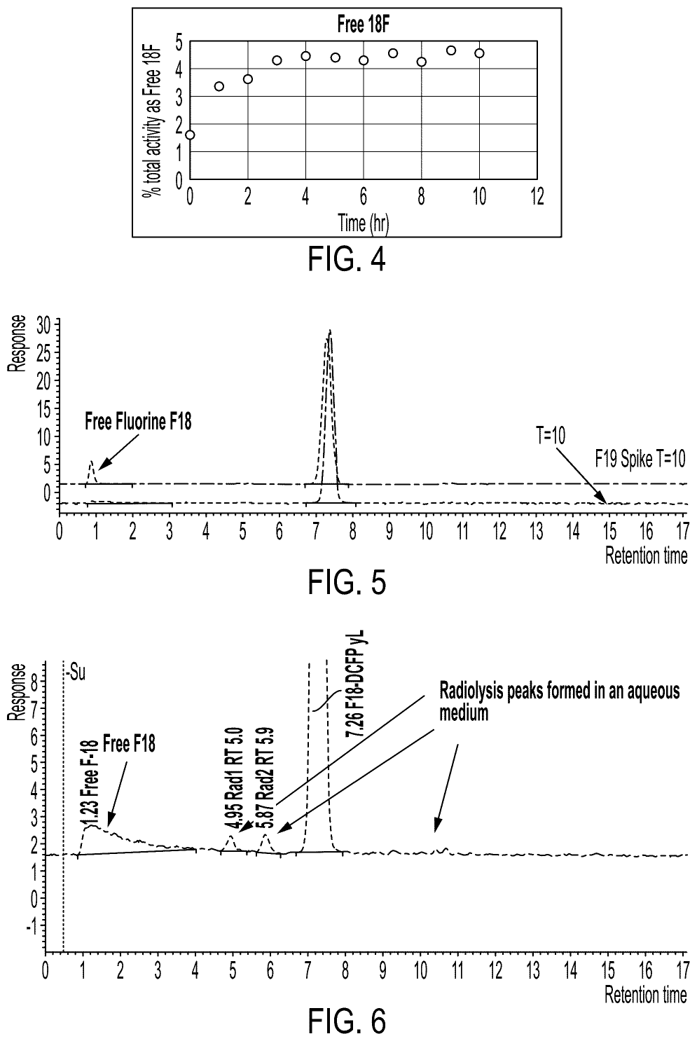

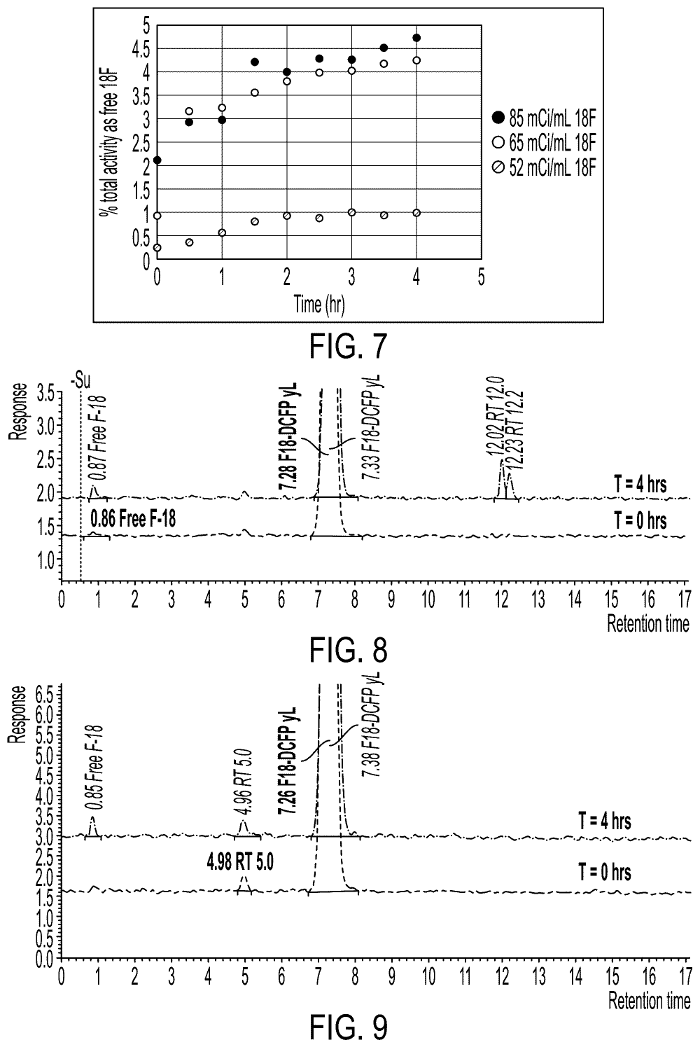

### Example 3. Ethanol as a Radioprotectant

[¹⁸F]DCFPyL samples were synthesized as in Example 1. Final product was at 325 mCi/mL at end of synthesis and maintained in 100% ethanol. Samples were taken every hour over 10 hrs and radiochemical and chemical purity determined by HPLC radiodetection or UV detection, respectively, as in Example 1. A time-dependent formation of free ¹⁸F was apparent (FIG. 4 and Table 1) with no additional radioactive impurities.

TABLE 1
Formation of free 18F in the presence of ethanol

| Solution Ethanol | T = 0 | T = 1 | T = 2 | T = 3 | T = 4 | T = 5 | T = 6 | T = 7 | T = 8 | T = 9 | T = 10 |
|---|---|---|---|---|---|---|---|---|---|---|---|
| 18F-DCFPyL | 98.37 | 96.66 | 96.38 | 95.71 | 95.60 | 95.58 | 96.73 | 96.42 | 95.76 | 95.31 | 95.45 |
| Free F-18 | 1.63 | 3.34 | 3.62 | 4.29 | 4.40 | 4.42 | 4.27 | 4.58 | 4.24 | 4.69 | 4.55 |

The estimation of free ¹⁸F was limited by the poor peak shape of the ¹⁸F coming off the HPLC column. This peak shape was improved by the addition of TEAF. This demonstrates the potential for underestimating free ¹⁸F ( ).

Table 2).

TABLE 2
Impact of adding TEAF to sample before HPLC analysis

| | | | | Peak Area | | Peak Heights | |
|---|---|---|---|---|---|---|---|
| | | +TEAF | | Free | 18F- | Free | 18F- |
| | 10 hr | 10 Hr | | 18F | DCFPyL | 18F | DCFPyL |
| 18F DCFPyL | 95.5 | 92.7 | T = 10 hr | 23.0 | 482.1 | 0.4 | 31.2 |
| Free 18F | 4.6 | 7.4 | T = 10 Hr + TEAF | 37.5 | 472.4 | 4.0 | 25.9 |

### Example 4. Formation of Radioactive Impurities in the Presence of Ethanol and Water

[¹⁸F]DCFPyL samples were synthesized as in Example 1. Final product was at 146.76 mCi/mL at end of synthesis and maintained in 36% ethanol in saline. Samples were taken every 30 minutes over 4 hrs and radiochemical and chemical purity determined by HPLC radiodetection or UV detection, respectively, as in Example 1. A time-dependent formation of free ¹⁸F was apparent (

Table 3) with several additional un-identified radioactive impurities.

TABLE 3
Percent total radioactive detected activity in 35% ethanol in saline

| 36% Ethanol | T = 0 hr | T = 0.5 hr | T = 1 hr | T = 1.6 hr | T = 2 hr | T = 2.5 hr | T = 3 hr | T = 3.5 hr | T = 4 hr |
|---|---|---|---|---|---|---|---|---|---|
| 18F-DCFPyL | 94.17 | 90.65 | 90.71 | 89.59 | 89.50 | 88.79 | 88.94 | 88.90 | 89.06 |
| Free F-18 | 4.68 | 7.73 | 7.69 | 8.78 | 8.67 | 9.66 | 9.49 | 9.56 | 9.27 |
| RT 5.0 | 0.50 | 0.65 | 0.48 | 0.61 | 0.77 | 0.62 | 0.70 | 0.59 | 0.71 |
| RT 5.9 | 0.64 | 0.97 | 1.11 | 1.03 | 1.05 | 0.93 | 0.87 | 0.95 | 0.96 |

RT = retention time of radioactive impurity

### Example 5. Radioactive Concentration Related Formation of Impurities in the Presence of Ethanol and Water

[¹⁸F]DCFPyL samples were synthesized as in Example 1. Final product radioactive concentration ranged from 52 to 85 mCi/mL at end of synthesis and were maintained in 20% ethanol in saline. Samples were taken every 30 minutes over 4 hrs and radiochemical and chemical purity determined by HPLC radiodetection or UV detection, respectively, as in Example 1. A time- and concentration-dependent formation of free ¹⁸F was apparent with several additional un-identified radioactive impurities (

Table 4).

TABLE 4
Percent total detected activity in 20% ethanol in saline

| Solution | T = 0 hr | T = 0.5 hr | T = 1 hr | T = 1.5 hr | T = 2 hr | T = 2.5 hr | T = 3 hr | T = 3.5 hr | T = 4 hr |
|---|---|---|---|---|---|---|---|---|---|
| 85 mCi/mL | | | | | | | | | |
| 18F-DCFPyL | 97.56 | 96.74 | 96.64 | 95.06 | 95.37 | 95.06 | 95.10 | 95.14 | 94.93 |
| Free F-18 | 2.10 | 2.92 | 2.98 | 4.22 | 4.00 | 4.29 | 4.26 | 4.51 | 4.73 |
| RT 5.0 | 0.34 | 0.34 | 0.38 | 0.41 | 0.39 | 0.31 | 0.37 | 0.35 | 0.34 |
| RT 5.9 | <QL | <QL | <QL | 0.31 | 0.24 | 0.35 | 0.27 | <QL | <QL |
| 65 mCi/mL | | | | | | | | | |
| 18F-DCFPyL | 98.75 | 96.43 | 96.15 | 95.78 | 95.91 | 95.36 | 95.64 | 95.44 | 95.50 |
| Free F-18 | 0.91 | 3.17 | 3.22 | 3.55 | 3.78 | 3.97 | 4.03 | 4.17 | 4.23 |
| RT 5.0 | 0.34 | 0.39 | 0.36 | 0.38 | 0.31 | 0.41 | 0.34 | 0.39 | 0.27 |
| RT 5.9 | <QL | <QL | 0.28 | 0.29 | <QL | 0.26 | <QL | <QL | <QL |
| 52 mCi/mL | | | | | | | | | |
| 18F-DCFPyL | 99.31 | 99.18 | 98.93 | 98.70 | 98.62 | 98.61 | 98.53 | 98.55 | 98.53 |
| Free F-18 | 0.23 | 0.36 | 0.56 | 0.80 | 0.91 | 0.86 | 0.99 | 0.94 | 0.98 |
| RT 5.0 | 0.46 | 0.47 | 0.51 | 0.50 | 0.47 | 0.53 | 0.48 | 0.52 | 0.49 |
| RT 5.9 | <QL | <QL | <QL | <QL | <QL | <QL | <QL | <QL | <QL |

RT = retention time of radioactive impurity

### Example 6. Formation of Radioactive Impurities in Ethanol and 100 mg/mL Ascorbic Acid

[18F]DCFPyL samples were synthesized as in Example 1. However, the peak collected from the HPLC was added to ascorbic acid pH 2.0 to give a 100 mg/mL final concentration. Solvent exchange was performed with ascorbic acid added to the washing solution at 100 mg/mL, and the final ethanol elution was diluted to a final ascorbic acid concentration of 100 mg/mL. Final product radioactive concentration was at 256 mCi/mL at end of synthesis and was maintained in ethanol. Samples were taken every 30 minutes over 4 hrs and radiochemical and chemical purity determined by HPLC radiodetection or UV detection respectively as in Example 1. Formation of free 18F was markedly suppressed with only 0.08% accumulating over 4 hrs (Table 5). However, radioactive impurities at retention time (RT) 12 and 12.2 minutes accumulated versus time in the presence of ascorbic acid (FIG. 8).

Table 5 This differs from samples collected in ethanol alone or ethanol in saline where high formation of free ¹⁸F over 4 hrs was observed. However, radioactive impurities at retention time (RT) 12 and 12.2 minutes accumulated versus time in the presence of ascorbic acid (FIG. 8).

TABLE 5
Percent total detected activity in 100 mg/mL ascorbic acid

| 256 mCi/mL | T = 0 | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL | 99.71 | 99.60 | 98.68 | 98.58 | 98.35 | 98.71 | 98.46 | 98.03 | 97.98 |
| Free ¹⁸F | 0.29 | 0.40 | 0.31 | 0.31 | 0.31 | 0.31 | 0.29 | 0.35 | 0.37 |
| RT 5.0 | <QL | <QL | 0.35 | 0.28 | 0.35 | <QL | <QL | <QL | <QL |
| RT 12.0 | <QL | <QL | 0.35 | 0.46 | 0.52 | 0.52 | 0.69 | 0.88 | 0.96 |
| RT 12.2 | <QL | <QL | 0.31 | 0.37 | 0.47 | 0.46 | 0.57 | 0.75 | 0.69 |

### Example 7. Formation of Radioactive Impurities in Ethanol and 50 mg/mL Ascorbic Acid

[¹⁸F]DCFPyL samples were synthesized as in Example 1. However, the final ethanol elution was diluted to a final ascorbic acid concentration of 50 mg/mL at pH 5.8. Final product radioactive concentration was 111 mCi/mL at end of synthesis and maintained in 20% ethanol. Samples were taken every 30 minutes over 4 hrs and radiochemical and chemical purity determined by HPLC radiodetection or UV detection respectively as in Example 1. Formation of free ¹⁸F was markedly suppressed with only 0.26% accumulating over 4 hrs (

Table 6). This differs from samples collected in ethanol alone or ethanol in saline where high formation of free ¹⁸F over 4 hrs was observed. Furthermore, impurities at RT 12 and 12.2 were not apparent, and impurities at RT 5 and 5.9 were also minimal (

Table 6).

TABLE 6
Percent total detected activity in 50 mg/mL ascorbic acid in final product vial

| | T = 0 | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL | 99.73 | 99.69 | 96.73 | 99.61 | 99.67 | 99.63 | 99.25 | 99.46 | 99.37 |
| Free ¹⁸F | <QL | <QL | <QL | <QL | <QL | <QL | 0.35 | 0.24 | 0.26 |
| RT 5.0 | 0.27 | 0.31 | 0.27 | 0.39 | 0.33 | 0.37 | 0.39 | 0.31 | 0.37 |
| RT 5.9 | <QL | <QL | <QL | 0.31 | 0.24 | 0.35 | 0.27 | <QL | <QL |

### Example 8. Formation of Radioactive Impurities in Ethanol and Range of Ascorbic Acid Concentrations

[¹⁸F]DCFPyL samples were synthesized as in Example 1. However, the final ethanol elution was diluted to a final ascorbic acid concentration of 0.5 to 50 mg/mL at pH 5.8. Final product radioactive concentrations were 99 to 139 mCi/mL at end of synthesis and were maintained in 20% ethanol. Samples were taken at time 0, 2, 4, 6 and 10 hrs and radiochemical and chemical purity determined by HPLC radiodetection or UV detection respectively as in Example 1. Formation of free ¹⁸F was inhibited in a concentration-dependent manner but markedly suppressed at all ascorbic acid concentrations tested over 10 hrs (.

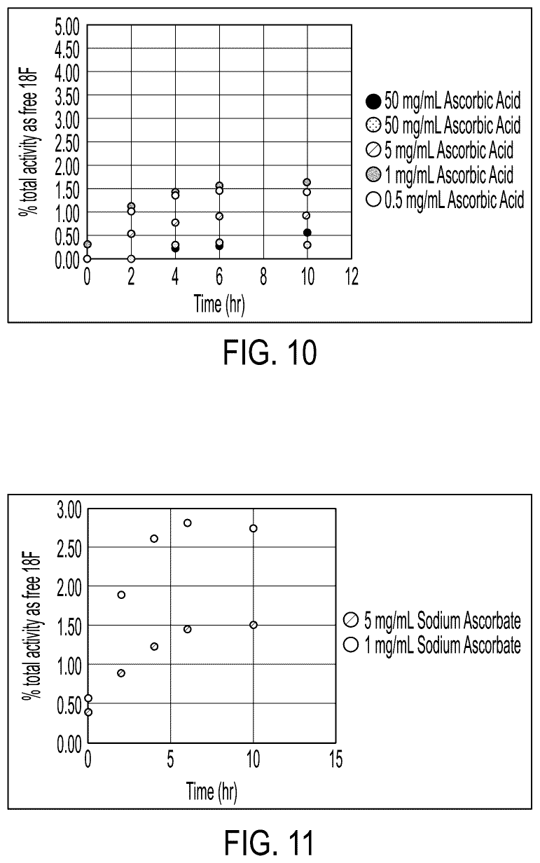

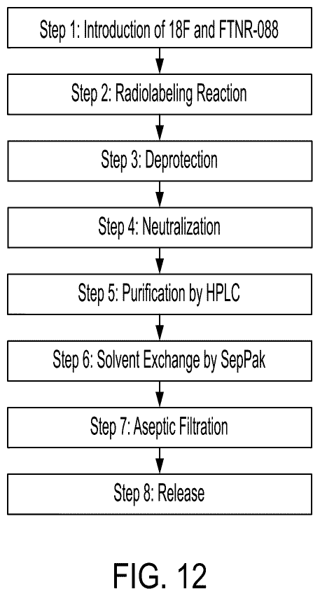

Table 7 and FIG. 10). This differs from samples collected in ethanol alone or ethanol in saline where high formation of free ¹⁸F over 4 hrs was observed. Furthermore, impurities at RT 12 and 12.2 and 5.9 were not apparent and an impurity at RT 5 was also minimal.

TABLE 7
Percent of total radioactivity detected in HPLC
separated samples with ascorbic acid concentration
from 0.5 to 50 mg/mL in the final product vial

| | EOS: | | | | |
|---|---|---|---|---|---|
| | 106.5 | mCi/mL | 50 mg/mL Ascorbic Acid | | |
| | Solution | | | | |
| | % RCP - with MPA (0.25% of main peak) | | | | |
| | Ascorbic Acid | | | | |
| | T = 0/1 | T = 3 | T = 4 | T = 6 | T = 10 |
| 18F-DCFPyL | 100.00 | 100.00 | 99.75 | 99.72 | 99.43 |
| Free F-18 | 0.00 | 0.00 | 0.25 | 0.28 | 0.57 |
| | EOS: | | | | |
| | 139.7 | mCi/mL | 50 mg/mL Ascorbic Acid | | |
| | Solution | | | | |
| | % RCP - with MPA (0.25% of main peak) | | | | |
| | Ascorbic Acid | | | | |
| | T = 0 | T = 2 hr | T = 4 hr | T = 6 hr | T = 10 hr |
| 18F-DCFPyL | 100.00 | 100.00 | 99.69 | 99.64 | 99.34 |
| Free F-18 | 0.00 | 0.00 | 0.31 | 0.36 | 0.30 |
| RT 4.8 | <MPA | <MPA | <MPA | <MPA | 0.36 |
| | EOS: | | | | |
| | 99.0 | mCi/mL | 5 mg/mL Ascorbic Acid | | |
| | Solution | | | | |
| | % RCP - with MPA (0.25% of main peak) | | | | |
| | Absorbic Acid | | | | |
| | T = 0 | T = 2 hr | T = 4 hr | T = 6 hr | T = 10 hr |
| 18F-DCFPyL | 100.00 | 99.45 | 99.23 | 99.09 | 99.05 |
| Free F-18 | 0.00 | 0.55 | 0.77 | 0.91 | 0.95 |
| | EOS: | | | | |
| | 107.0 | mCi/mL | 1 mg/mL Ascorbic Acid | | |
| | Solution | | | | |
| | % RCP - with MPA (0.25% of main peak) | | | | |
| | Ascorbic Acid | | | | |
| | T = 0 | T = 2 hr | T = 4 hr | T = 6 hr | T = 10 hr |
| 18F-DCFPyL | 99.68 | 98.88 | 98.56 | 98.43 | 98.36 |
| Free F-18 | 0.32 | 1.12 | 1.44 | 1.57 | 1.64 |
| | EOS: | | | | |
| | 105.8 | mCi/mL | 0.5 mg/mL Ascorbic Acid | | |
| | Solution | | | | |
| | % RCP - with MPA (0.25% of main peak) | | | | |
| | Ascorbic Acid | | | | |
| | T = 0 | T = 2 hr | T = 4 hr | T = 6 hr | T = 10 hr |
| 18F-DCFPyL | 100.00 | 98.99 | 98.64 | 98.56 | 98.58 |
| Free F-18 | 0.00 | 1.01 | 1.36 | 1.44 | 1.42 |

### Example 9. Formation of Radioactive Impurities in the Presence of Ethanol and Range of Sodium Ascorbate Concentrations

[¹⁸F] DCFPyL samples were synthesized as outlined in Example 1. However, the final ethanol elution was diluted to a final sodium ascorbate concentration of 1, 2.5 or 5 mg/mL. Final product radioactive concentrations at end of synthesis were measured and were maintained in 20% ethanol. Samples were taken at time 0, 2, 4, 6 and 10 hr and radiochemical and chemical purity determined by HPLC radio-detection or UV detection respectively as described in Example 1. Formation of free ¹⁸F was inhibited in a concentration dependent manner and suppressed at all ascorbic acid concentrations tested over 10 hrs. This differs from samples collected in ethanol alone or ethanol in saline where high formation of free ¹⁸F over 4 hrs was seen.

Comparison of sodium ascorbate with ascorbic acid indicates ascorbic acid was more effective at inhibiting de-fluorination of ¹⁸F-DCFPyL. Furthermore, impurities at RT 12, 12.2, 5.9 and 5.0 were not apparent (Table 8).

TABLE 8
Percent of total radioactivity detected with sodium ascorbate
concentration of 1 or 5 mg/mL in the final product vial

| | EOS: | | | | |
|---|---|---|---|---|---|
| | 114.6 | mCi/mL | 5 mg/mL Sodium Ascorbate | | |
| | Solution | | | | |
| | % RCP - with MPA (0.25% of main peak) | | | | |
| | Ascorbate | | | | |
| | T = 0.5 | T = 2 hr | T = 4 hr | T = 6 hr | T = 10 hr |
| 18F-DCFPyL | 99.61 | 99.11 | 98.77 | 98.55 | 98.49 |
| Free F-18 | 0.39 | 0.89 | 1.23 | 1.45 | 1.51 |
| | EOS: | | | | |
| | 111.5 | mCi/mL | 1 mg/mL Sodium Ascorbate | | |
| | Solution | | | | |
| | % RCP - with MPA (0.25% of main peak) | | | | |
| | Ascorbate | | | | |
| | T = 0 | T = 2 hr | T = 4 hr | T = 6 hr | T = 10 hr |
| 18F-DCFPyL | 99.43 | 98.11 | 97.39 | 97.19 | 97.26 |
| Free F-18 | 0.57 | 1.89 | 2.61 | 2.81 | 2.74 |

### Example 10. Formation of Radioactive Impurities in the Presence of Ethanol and Sodium Citrate

[¹⁸F] DCFPyL samples were synthesized as outlined in Example 1. However, the final ethanol elution was diluted to a final sodium citrate concentration of 20 mg/mL. Final product radioactive concentrations was 104.8 mCi/mL at end of synthesis and were maintained in 20% ethanol. Samples were taken at time 0, 2 and 4 hrs and radiochemical and chemical purity determined by HPLC radiodetection or UV detection respectively as described in Example 1. Formation of free ¹⁸F occurred rapidly with 10.22% present at time 0 and increasing to 20.65% at 4 hr (Table 9). This is similar to samples collected in ethanol alone or ethanol in saline where high formation of free ¹⁸F over 4 hrs was seen. The majority of radio-impurity formed was free ¹⁸F.

TABLE 9
Percent of total radioactivity detected in HPLC
separated samples with sodium citrate concentration
of 20 mg/mL in the final product vial

| | EOS: | | |
|---|---|---|---|
| | 104.8 | mCi/mL | 20 mg/mL Sodium Citrate |
| | Solution | | |
| | % RCP - with MPA (0.25% of main peak) | | |
| | Citrate | | |
| | T = 0 | T = 2 hr | T = 4 hr |
| 18F-DCFPyL | 89.78 | 81.14 | 78.68 |
| Free F-18 | 10.22 | 18.24 | 20.65 |
| RT 3.4 | <MPA | 0.30 | 0.32 |
| RT 3.7 | <MPA | 0.31 | 0.35 |

### Example 11. Ascorbic Acid pH and Sodium Ascorbate pH as a Function of Concentration

Solutions at different concentrations ranging from 50 mg/mL to 0.5 mg/mL of Ascor USP ascorbic acid in SWFI and 50 mg/mL to 0.5 mg/mL of sodium ascorbate from Spectrum in saline were prepared. The pH was measured using a Corning pH meter calibrated using 4.0 and 7.0 pH buffer and Macherey-Nagel pH paper with a range of 2.0 to 9.0. The pH range of the Ascor USP ascorbic acid solution used is 5.6 to 6.6, so aliquots of 50 mg/mL, 5 mg/mL, 2.5 mg/mL and 1 mg/mL of sodium ascorbate from Spectrum in saline were pH adjusted with concentrated hydrochloric acid to achieve a pH close to the range (Table 10). On a different day, another set of 2.5 mg/mL sodium ascorbate in saline neat and pH adjusted with concentrated hydrochloric acid was also prepared. The pH of this set was measured using a Corning pH meter calibrated using 4.0 and 7.0 pH buffer, Macherey-Nagel pH paper with a range of 2.0 to 9.0, and EMD pH paper with a range of 2.0 to 9.0 (Table 11).

TABLE 10
pH results measured with pH meter and pH paper

| | Corning | Macherey- |
|---|---|---|
| | pH | Nagel pH |
| Sample | Meter | Paper |
| SWFI | 6.0 | 7.0 |
| Saline | 5.2 | 7.0 |
| 50 mg/mL ascorbic acid in SWFI | 5.7 | 5.5 |
| 5 mg/mL ascorbic acid in SWFI | 5.7 | 6.0 |
| 1 mg/mL ascorbic acid in SWFI | 5.8 | 6.0 |
| 0.5 mg/mL ascorbic acid in SWFI | 5.9 | 6.5 |
| 50 mg/mL sodium ascorbate in saline | 7.2 | 8.5 |
| 5 mg/mL sodium ascorbate in saline | 7.2 | 7.5 |
| 1 mg/mL sodium ascorbate in saline | 6.5 | 7.0 |
| 0.5 mg/mL sodium ascorbate in saline | 6.3 | 7.0 |
| 5 mg/mL sodium ascorbate in saline | 5.1 | 5.5 |
| pH adjusted | | |
| 50 mg/mL sodium ascorbate in saline | 6.0 | 6.0 |
| pH adjusted | | |
| 1 mg/mL sodium ascorbate in saline | 5.0 | 6.0 |
| pH adjusted | | |
| 2.5 mg/mL sodium ascorbate in saline | 6.8 | 7.0 |
| 2.5 mg/mL sodium ascorbate in saline | 5.8 | 7.0 |
| pH adjusted | | |

TABLE 11
pH results measured with pH meter and pH paper

| | Corning | Macherey- | EMD |
|---|---|---|---|
| | pH | Nagel pH | pH |
| Sample | Meter | Paper | paper |
| 2.5 mg/mL sodium ascorbate in saline | 7.3 | 7.0 | 5.5 |
| 2.5 mg/mL sodium ascorbate in saline | 5.9 | 6.0 | 5.0 |
| pH adjusted | | | |

### Example 12. Formulations of [¹⁸F]DCFPyL

A desirable formulation of [¹⁸F]DCFPyL is provided in (Table 12). The formulation is 0.9% sodium chloride, USP, with up to 7.89% ethanol w/v, such as 3% ethanol. A schematic of a Trasis ALLinONE (AIO) synthesis module with reagents and reagent positions is provided in FIG. 13. Generally, there can be an upper limit of 80 mCi/mL radioactive concentration at end of synthesis (EOS). In order to improve drug supply and/or allow for more doses per lot (lot size may be limited by the maximum drug concentration product vial size (e.g., 50 mL)), a product having a higher radioactive concentration, such as >120 mCi/mL at EOS, can be beneficial. In addition, the major radioactive degradation product observed is free ¹⁸F and is formed through radiolysis of [¹⁸F]DCFPyL. Minimizing radiolysis can be beneficial.

TABLE 12
[18F]DCFPyL formulation

| | Quantity | Role in | Quality |
|---|---|---|---|
| Component | per Dose | Formulation | Standard |
| ¹⁸F-DCFPyL | 9 mCi @TOA ≤4 μg | Active drug | GMP |
| | chemical mass^{a} | substance | |
| Ethanol 100% | 1 to 7.89% w/v^{b} | Eluent used in drug | ACS |
| | | substance formulation | ISO |
| | | | Eur Ph Reagent |
| Normal | 92 to 99% w/v^{c} | Eluent/diluent used | USP |
| Saline | | in drug substance | ACS |
| | | formulation | ISO |
| | | | Eur Ph Reagent |

^{a}TOA = Time of Administration
^{b}Depends on batch's specific activity at the end of the synthesis (EOS) and a lag time between EOS and TOA
^{c}The dilution with normal saline post-production is performed to achieve a radiochemical concentration ≤80 mCi/mL at EOS. The degree of dilution varies depending on a starting batch scale (1 Ci to 10 Ci) and process yield.

Studies were undertaken to examine the addition of a radioprotectant to formulations of [¹⁸F]DCFPyL and success of minimizing drug substance radiolysis. While ascorbic acid has been demonstrated to quench high radioactive concentration-induced radiolysis of certain PET products (e.g., US20200222562), its effectiveness with respect to preparations of [¹⁸F]DCFPyL with specific desired features has not been previously evaluated. A comprehensive analysis of the ability of ascorbic acid to stabilize [¹⁸F]DCFPyL, such as at higher radioactive concentrations (e.g, >120 mCi/mL), and the impact on such desirable features of formulations thereof was undertaken.

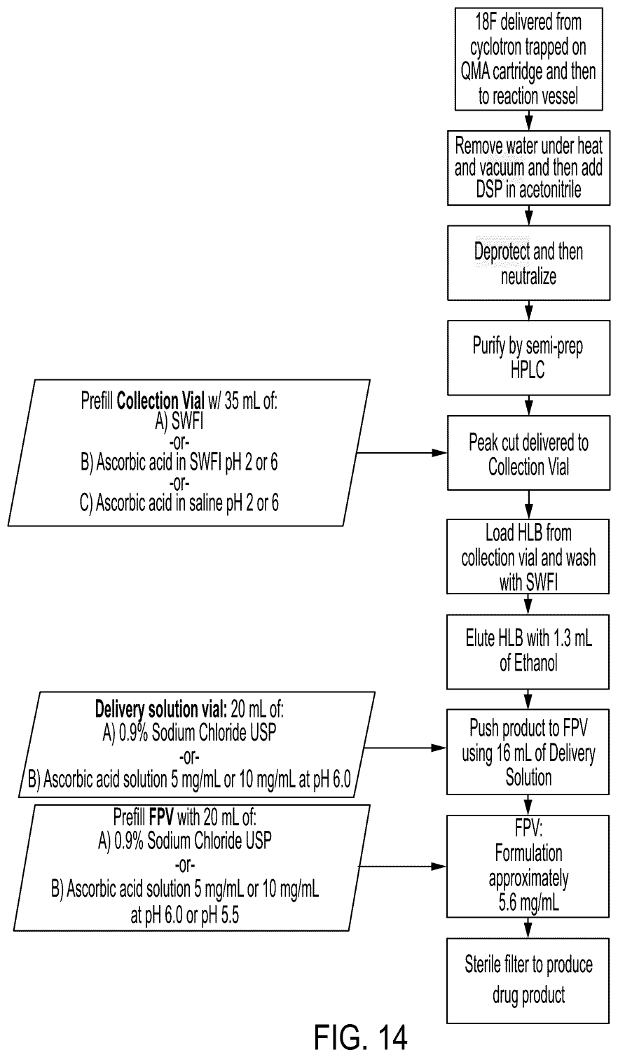

A flow diagram indicating where ascorbic acid was introduced in the production process is shown in FIG. 14. In an embodiment, ascorbic acid may be delivered to the final product vial (FPV) as part of the product delivery from the HLB solvent exchange cartridge (ascorbic acid added to the Delivery Solution Vial; position F on the AIO, FIG. 13) and/or by direct introduction into the FPV. The addition of ascorbic acid to the collection vial used to receive the peak cut from the semi-prep purification also provides a path to stabilizing the product prior to the solvent exchange step, in another embodiment. In another embodiment, ascorbic acid can be used to precondition a cartridge, such as a HLB cartridge, used to perform solvent exchange.

### HPLC Radiochemical and Chemical Impurity Assay

Analytical HPLC method was used. The method employs UV detection at 264 nm for chemical impurities, and an Eckert & Ziegler model B-FC-1000 gamma detector for radiochemical assays.

### [¹⁸F]DCFPyL Specification

The specifications of a desirable [¹⁸F]DCFPyL product are provided in Table 15. A general test plan is also provided (Table 16), though in some cases the test timepoints were modified as indicated.

TABLE 15
[18F]DCFPyL product specification

| | Quantity | Role in | Quality |
|---|---|---|---|
| Component | per Dose | Formulation | Standard |
| ¹⁸F-DCFPyL | 9 mCi @TOA^{a} ≤4 μg | Active drug | GMP |
| | chemical mass^{b} | substance | |
| Ethanol 100% | 1 to 7.89% wt/v | Eluent used in drug | ACS |
| | | substance | ISO |
| | | formulation | Eur Ph Reagent |
| 0.9% Sodium | 91-98% wt/v^{c} | Eluent/Diluent used | USP |
| Chloride, USP | | in drug substance | ACS |
| | | formulation | ISO |
| | | | Eur Ph Reagent |
| Sodium Ascorbate | mg/mL | Radioprotectant | USP |
| | 0.628% wt/v | | ACS |
| | | | ISO |
| | | | Eur Ph Reagent |
| 1N Hydrochloric | 0.0002% wt/v | pH adjustment | USP |
| acid, USP | | | |

^{a}TOA = Time of administration
^{b}Depends on batch's specific activity at the end of the synthesis (EOS) and a lag time between EOS and TOA
^{c}Formula A (Ascorbate) drug product is not diluted.

TABLE 16
Test plan

| | Huntsman Method | |
|---|---|---|
| Test | Reference | Timepoint |
| Appearance | Q015 | 0 |
| Membrane Filter Integrity | Bubble point pressure test | 0 |
| pH | Q014 | 0 and 10 hrs |
| Radiochemical Purity | See Attachment 1 | 0, 2, 4, 6 and 10 hrs |
| Radiochemical Identity | See Attachment 1 | 0 |
| Radionuclidic Identity | Q005 | 0 |
| by Half-life | | |
| Chemical Purity | See Attachment 1 | 0, 10 hours |
| Radioactive Concentration | M526 | 0 |
| Residual Solvent Analysis | Q003 | 0 |
| Specific Activity at Expiration | Q526 | 0 |
| Ascorbic Acid | See Attachment | 0 and 10 hours |

### [¹⁸F]DCFPyL Studies Performed at 18F Starting Activity of Approximately 2 Ci

The feasibility of stabilizing [¹⁸F]DCFPYL using ascorbic acid added to the 1) collection vial used to receive the product peak cut from the semi prep purification step, 2) to precondition the HLB cartridge used to perform solvent exchange, and to the 3) final product vial, was assessed. In order to achieve radioactive concentrations of approximately 120 mCi/mL, the labeled product was eluted from the HLB cartridge used for solvent exchange and delivered using a Nitrogen push to the receiving vial which was preloaded with a minimum of 1.3 mL of formulation matrix (0.9% sodium chloride, USP, or ascorbic acid, USP). The product was then assayed by Capintec and diluted to achieve desired radioactive concentration. This results in a product having a higher ethanol level (approximately 20%) than typically used clinically (e.g., around 3%). While ethanol is known to provide some protection against radiolysis, when compared to an ethanol control, it provided an initial assessment of the impact of ascorbic acid on stabilization.

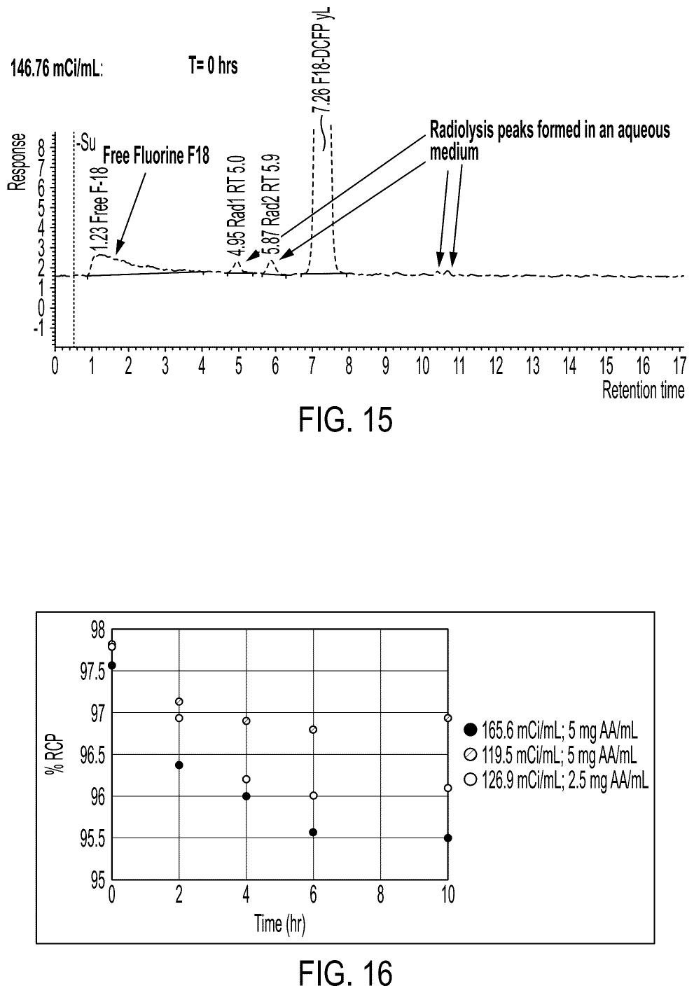

A control lot was produced without incorporation of ascorbic acid and had a final radiochemical concentration of 147 mCi/mL and a 29.8% ethanol level. The product was sampled every 30 minutes and assayed with an extended gradient added to allow detection of hydrophobic impurities. The product was delivered from the HLB into a vial containing 1.3 mL of 0.9% sodium chloride, USP. The product was assayed and diluted to achieve the desired radioactive concentration with 0.9% sodium chloride, USP. The initial radiochemical purity (RCP) was 94% and the T=4 hour RCP was 89%, well below the desirable ≥95% RCP product specification. In addition to free ¹⁸F, there were two additional radioactive impurities that elute before the [¹⁸F]DCFPYL peak at initial time point chromatogram but do not appear to grow over time. These may be process impurities as opposed to ongoing radiolytic degradation. There are two additional peaks below the reportable limit of 0.3% w/w that elute after the [¹⁸F]DCFPYL product peak. Data are provided in Table 17. The T=0 hour chromatogram is provided in FIG. 15.

A lot was produced with 35 mL of 100 mg/mL, pH 2.0 ascorbic acid solution preloaded into the collection vial. The HLB cartridge was also rinsed with 5 mL of the 100 mg/mL, pH 2.0 ascorbic acid solution. The [¹⁸F]DCFPYL was delivered from the HLB into a vial containing 1.3 mL of 0.9% sodium chloride, USP. The product was assayed and diluted to the desired radioactive concentration with 0.9% sodium chloride, USP. Samples were taken every 30 minutes and assayed. For radiochemical purity HPLC analysis the sample had tetraethylammonium fluoride, 44 mg/mL, added to reduce column retention and sharpen the free ¹⁸F peak allowing for better quantitation. The product had a radiochemical concentration of 256 mCi/mL. The initial RCP was 99.7%, and the free ¹⁸F impurity was 0.29%. Only one of the two impurities, seen in the control lot, eluting before the [¹⁸F]DCFPYL peak was sporadically present ranging from <LLQ to 0.35%, but were not seen consistently at reportable levels (>0.3%). The RCP was 98.0% at T=4 hours. Data for this lot are provided in Table 18. This clearly shows that radiolysis was greatly reduced when ascorbic acid was added to the collection vial.

A lot was produced with 35 mL of 100 mg/mL, pH 2.0 ascorbic acid solution preloaded into the collection vial. The HLB cartridge was also rinsed with 5 mL of the 100 mg/mL, pH 2.0 ascorbic acid solution. Finally, the [¹⁸F]DCFPYL was collected in a vial preloaded with 50 mg/mL of pH 5.8 ascorbic acid solution, assayed by Capintec and diluted to the desired concentration with 50 mg/mL of pH 5.8 ascorbic acid solution. The product was sampled every 30 minutes and assayed. For radiochemical purity HPLC analysis the sample had tetraethylammonium fluoride, 44 mg/mL, added to reduce column retention and sharpen the free ¹⁸F peak allowing for better quantitation. The product had a radiochemical concentration of 110 mCi/mL and a 24% (w/w) ethanol level. The initial RCP was 99.7%, and the ¹⁸F impurity was <LLQ. The two impurities eluting before the [¹⁸F]DCFPYL peak, seen in the control lot, were present at levels ranging from <LLQ to 0.37%, but were not consistently at reportable levels (>0.3%). The RCP remained above 99% at T=4 hours. Data for this lot are provided in Table 19. This clearly shows that radiolysis was greatly reduced with the use of ascorbic acid in the collection vessel, wash solution and in the FPV. However, the contribution of ethanol to the stabilization of [¹⁸F]DCFPYL is unknown.

TABLE 17
Radiolysis of 147 mCi of [¹⁸F]DCFPYL/mL in 0.9% sodium chloride, USP

| | T = 0 | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL | 94.17 | 90.65 | 90.71 | 89.59 | 89.50 | 88.79 | 88.94 | 88.90 | 89.06 |
| Free ¹⁸F | 4.68 | 7.73 | 7.69 | 8.78 | 8.67 | 9.66 | 9.49 | 9.56 | 9.27 |
| RT 5.0 | 0.50 | 0.65 | 0.48 | 0.61 | 0.77 | 0.62 | 0.70 | 0.59 | 0.71 |
| RT 5.9 | 0.64 | 0.97 | 1.11 | 1.03 | 1.05 | 0.93 | 0.87 | 0.95 | 0.96 |

TABLE 18
Radiolysis of 256 mCi of [¹⁸F]DCFPYL/mL in in 0.9% sodium chloride, USP^{a}

| 80 mCi/mL | T = 0 | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL | 99.71 | 99.60 | 98.68 | 98.58 | 98.35 | 98.71 | 98.46 | 98.03 | 97.98 |
| Free ¹⁸F | 0.29 | 0.40 | 0.31 | 0.31 | 0.31 | 0.31 | 0.29 | 0.35 | 0.37 |
| RT 5.0 | <QL | <QL | 0.35 | 0.28 | 0.35 | <QL | <QL | <QL | <QL |
| RT 12.0 | <QL | <QL | 0.35 | 0.46 | 0.52 | 0.52 | 0.69 | 0.88 | 0.96 |
| RT 12.2 | <QL | <QL | 0.31 | 0.37 | 0.47 | 0.46 | 0.57 | 0.75 | 0.69 |

^{a}The collection vial was preloaded with 35 mL of 100 mg/mL, pH 2.0 ascorbic acid and receives approximately 5 mL in the peak cut. The HLB cartridge was washed with 5 mL of 100 mg/mL, pH 2.0 ascorbic acid to leave it protected with the radical scavenger solution, and the product was delivered from the HLB into a vial containing 1.3 mL of 0.9% sodium chloride, USP. The product was assayed and diluted to the desired radioactive concentration with 0.9% sodium chloride, USP.

TABLE 19
Radiolysis of 110 mCi of [¹⁸F]DCFPYL/mL in 50 mg/mL pH 5.8 ascorbic acid^{a}

| 80 mCi/mL | T = 0 | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL | 99.73 | 99.69 | 96.73 | 99.61 | 99.67 | 99.63 | 99.25 | 99.46 | 99.37 |
| Free ¹⁸F | <QL | <QL | <QL | <QL | <QL | <QL | 0.35 | 0.24 | 0.26 |
| RT 5.0 | 0.27 | 0.31 | 0.27 | 0.39 | 0.33 | 0.37 | 0.39 | 0.31 | 0.37 |
| RT 5.9 | <QL | <QL | <QL | 0.31 | 0.24 | 0.35 | 0.27 | <QL | <QL |

^{a}The collection vial was preloaded with 35 mL of 100 mg/mL, pH 2.0 ascorbic acid and receives approximately 5 mL in the peak cut. The HLB cartridge was washed with 5 mL of 100 mg/mL, pH 2.0 ascorbic acid to leave it protected with the radical scavenger solution, and the product was delivered from the HLB into a vial containing 1.3 mL of 50 mg/mL, pH 5.8 ascorbic acid. The product was assayed and diluted to the desired radioactive concentration with 50 mg/mL, pH 5.8 ascorbic acid.

### [¹⁸F]DCFPyL Studies Performed at 18F Starting Activities (>10 Ci)

The stability of [¹⁸F]DCFPYL injection was studied at activities greater than the current product limit of 80 mCi/mL. Ascorbic acid was added as a radioprotectant to the FPV and/or to the “delivery solution vial” (Position F, FIG. 13), or was not incorporated in lot production as indicated in Table 20. A listing of solution compositions for these studies is provide in Table 21. Starting activity for the labelling reaction was targeted at 15 Ci with the goal of producing product at radioactive concentrations>120 mCi/mL.

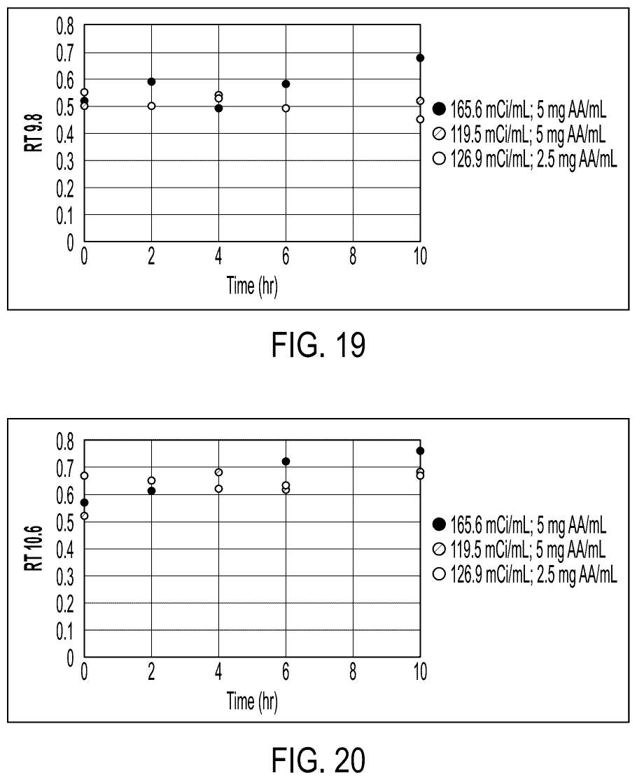

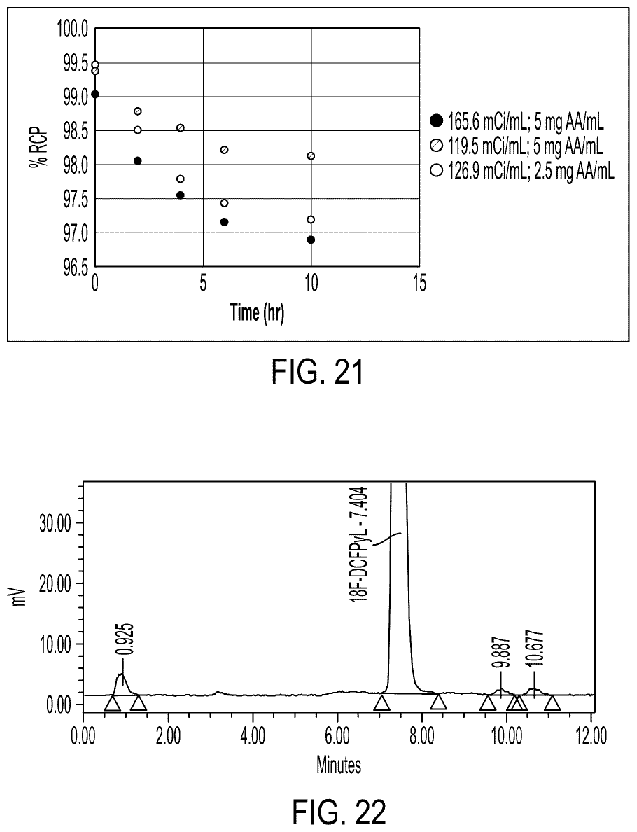

Three lots were produced incorporating pH 6.0 ascorbic acid into the final product vial (FPV). The lots were prepared using ¹⁸F starting activities of 17.6 Ci, 13.5 Ci and 13.4 Ci which resulted in final activity concentrations of 166 mCi/mL, 120 mCi/mL and 127 mCi/mL, respectively. Samples were assayed using the radiochemical HPLC assay at 0, 2, 4, 6 and 10 hours post end of synthesis (EOS). The T=0, T=4 and T=10 hour results for Lot 220816PyL, Lot 220817PyL, and Lot 220818PyL are provided in Table 22. Plots of the results for RCP, ¹⁸F, peaks at retention times (RT) of 6.0, 9.8 and 10.6 minutes are provided in FIGS. 16-FIG. 20, respectively, including additional timepoint data not listed in Table 22.

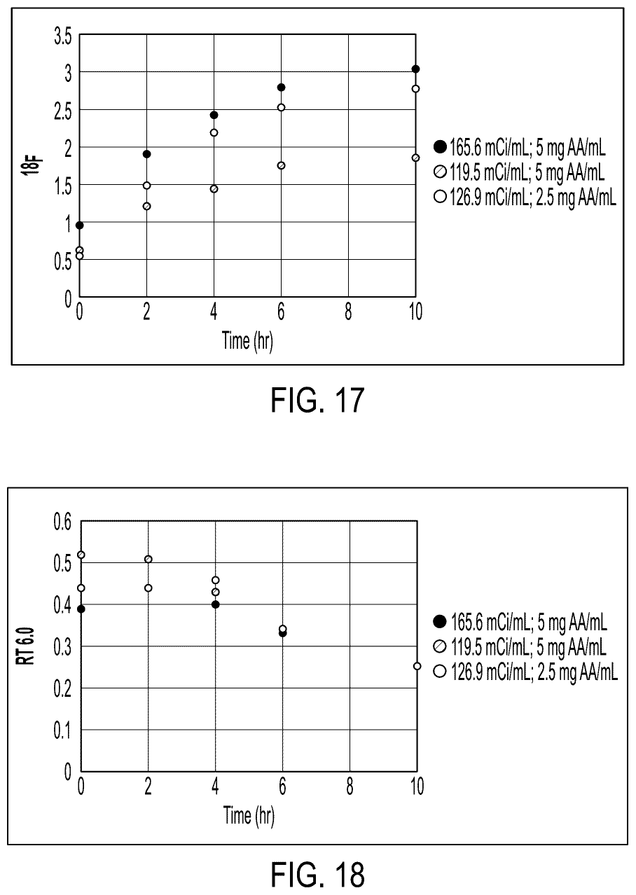

The data and plot for total RCP as a function of time indicates [¹⁸F]DCFPYL is more stable at lower radioactive concentration (T=10 hour RCP of 95.5% and 96.9% at 166 and 120 mCi/mL, respectively) when the ascorbic acid level is at 5.6 mg/mL, while decreasing the ascorbic acid concentration to 2.8 mg/mL for formulations at approximately the same radiochemical concentration (120 to 127 mCi/mL) results in lower RCP (96.9% versus 96.1% RCP), FIG. 16. The lot formulated at 127 mCi/mL in 2.8 mg/mL, pH 6.0 ascorbic acid, had slightly better stability (approximately 0.5% RCP difference) when compared to the lot formulated in 5.6 mg/mL, pH 6.0 ascorbic acid (165 mCi/mL), consistent with greater radiolysis at higher radioactive concentration, FIG. 16. The same lot had slightly poorer stability (approximately 0.5% difference) when compared to the lot formulated in 5.6 mg/mL, pH 6.0 ascorbic acid but having similar radioactive concentration (120 mCi/mL). This is consistent with the lower concentration of ascorbic acid in the formulation providing less radioprotection and is also evident from the plot of 18F formation for the lots, FIG. 17.

The plots for the peaks at retention times of 6.0, 9.8 and 10.6 minutes range between 0.4 and 0.6% at T=0 minutes and show small changes (<0.2%) from their initial levels over the 10 hour course of the study. A representative chromatogram is provided in FIG. 22. The lack of significant change from T=0 to T=10 hours may indicate these three peaks are process impurities. Overall, the three lots did not achieve the desired RCP (>99%) at T=0 hours, with the lower RCP results being driven potentially by the presence of process impurities. However, the lots do demonstrate addition of ascorbic acid at both 2.8 and 5.6 mg/mL to the FPV stabilizes [¹⁸F]DCFPYL degradation even at concentration as high as 165 mCi/mL.

TABLE 20
Position of ascorbic acid additions for FPV ascorbic acid studies

| | | Lot | Lot | Lot | Lot | Lot |
|---|---|---|---|---|---|---|
| Vial | | 220816PyL | 220817PyL | 220818PyL | 220819PyL | 220908PyL |
| Collection | Ascorbic | − | − | − | − | − |
| vial | Acid | | | | | |
| (Position I, | Saline | − | − | − | − | − |
| FIG. 13) | SWFI | + | + | + | + | + |
| Delivery | Ascorbic | − | − | − | + | N/A |
| solution vial | Acid | | | | | |
| (Position F, | Saline | + | + | + | + | N/A |
| FIG. 13) | SWFI | − | − | − | − | N/A |
| FPV | Ascorbic | + | + | + | + | N/A |
| | Acid | | | | | |
| | Saline | + | + | + | + | N/A |
| | SWFI | − | − | − | − | N/A |

TABLE 21
Ascorbic acid, pH 6 added to the FPV

| | Lot | Lot | Lot | Lot | Lot |
|---|---|---|---|---|---|
| Vial | 220816PyL | 220817PyL | 220818PyL | 220819PyL | 220908PyL |
| Collection vial | 35 mL of SWFI | 35 mL of SWFI | 35 mL of SWFI | 35 mL of SWFI | 35 mL of SWFI |
| (Position I, | | | | | |
| FIG. 13) | | | | | |
| Delivery | Fill: 20 mL of | Fill: 20 mL of | Fill: 20 mL of | Fill: 20 mL of | not applicable |
| solution vial | 0.9% saline | 0.9% saline | 0.9% saline | 5 mg/mL | |
| (Position F, | Delivery: 16 | Delivery: 16 | Delivery: 16 | ascorbic acid in | |
| FIG. 13) | mL to FPV | mL to FPV | mL to FPV | 0.9% saline | |
| | | | | Delivery: 16 | |
| | | | | mL to FPV | |
| FPV | 20 mL of 10 | 20 mL of 10 | 20 mL of 5 | 20 mL of 5 | 20 mL of 0.9% |
| | mg/mL ascorbic | mg/mL ascorbic | mg/mL ascorbic | mg/mL ascorbic | saline |
| | acid, pH 6 in | acid, pH 6 in | acid, pH 6 in | acid, pH 6 in | |
| | 0.9% saline | 0.9% saline | 0.9% saline | 0.9% saline | |
| Ascorbic acid | 5 mg/mL^{a} | 5 mg/mL^{a} | 2.8 mg/mL^{b} | 5 mg/m^{a}L | not applicable |
| concentration | | | | | |
| after delivery | | | | | |
| of all | | | | | |
| components | | | | | |

^{a}16 mL saline plus 20 mL of 10 mg/mL ascorbic acid = (20 mL × 10 mg/mL)/36 mL = 5.6 mg/mL ascorbic acid
^{b}16 mL saline plus 20 mL of 5 mg/mL ascorbic acid = (20 mL × 5 mg/mL)/36 mL = 5.6 mg/mL ascorbic acid

TABLE 22
Results for initial lots examining the addition of ascorbic acid to [¹⁸F]DCFPYL formulations

| | Lot 220816PyL | Lot 220817PyL | Lot 220818PyL | Lot 220819PyL | Lot 220908PyL |
|---|---|---|---|---|---|
| Starting Activity | 17.6 Ci | 13.5 Ci | 13.4 Ci | 13.7 Ci | 13.6 Ci |
| mCi/mL | 166 | 120 | 127 | 134 | 166 |
| Collection Vial | SWFI | SWFI | SWFI | SWFI | n/a^{a} |
| Delivery Solution | 0.9% sodium | 0.9% sodium | 0.9% sodium | 5 mg/mL pH 6.0 | n/a^{a} |
| Vial | chloride, USP | chloride, USP | chloride, USP | in 0.9% sodium | |
| | | | | chloride, USP | |
| FPV | 10 mg/mL, pH 6.0 | 10 mg/mL, pH 6.0 | 5 mg/mL, pH 6.0 | 5 mg/mL, pH 6.0 | n/a^{a} |
| | in 0.9% sodium | in 0.9% sodium | in 0.9% sodium | in 0.9% sodium | |
| | chloride, USP | chloride, USP | chloride, USP | chloride, USP | |
| Product ascorbic | 5.6 mg/mL, pH 6.0 | 5.6 mg/mL, pH 6.0 | 2.8 mg/mL, pH 6.0 | 5 mg/mL, pH 6.0 | n/a^{a} |
| acid concentration | | | | | |

| | T = 0 | T = 4 | T = 10 | T = 0 | T = 4 | T = 10 | T = 0 | T = 4 | T = 10 | T = 0 | T = 4 | T = 10 | T = 0 | T = 4 | T = 10 |
|---|---|---|---|---|---|---|---|---|---|---|---|---|---|---|---|
| RCP | 97.6 | 96.0 | 95.5 | 97.8 | 96.9 | 96.9 | 97.8 | 96.2 | 96.1 | 97.9 | 97.0 | nt^{b} | 97.6 | 81.4 | nt^{b} |
| 18F | 0.95 | 2.42 | 3.05 | 0.62 | 1.44 | 1.86 | 0.53 | 2.19 | 2.78 | 0.52 | 1.59 | nt^{b} | 1.12 | 11.07 | nt^{b} |
| RT 6.0 | 0.39 | 0.40 | 0.30 | 0.52 | 0.43 | 0.30 | 0.44 | 0.46 | 0.30 | 0.46 | 0.38 | nt^{b} | 0.64 | 2.13 | nt^{b} |
| RT 9.8 | 0.52 | 0.49 | 0.68 | 0.50 | 0.54 | 0.52 | 0.55 | 0.53 | 0.45 | 0.51 | 0.48 | nt^{b} | 1.08 | 1.99 | nt^{b} |
| RT 10.6 | 0.57 | 0.61 | 0.76 | 0.52 | 0.68 | 0.68 | 0.67 | 0.62 | 0.67 | 0.62 | 0.52 | nt^{b} | <LLQ | 0.72 | nt^{b} |

^{a}n/a = not applicable
^{b}nt = not tested

Addition of ascorbic acid (5 mg/mL) to the saline vial (position F, FIG. 13) that is used to deliver the product from the HLB cartridge to the final product vial (FPV) in addition to adding ascorbic acid (5 mg/mL) to the 20 mL of saline prefilled into the FPV was examined with high [¹⁸F]DCFPyL. Lot 220819PyL was produced with a starting activity of 13.7 Ci, and the radioactive concentration of the product was 134 mCi/mL at EOS. The RCP and free 18F results for Lot 220819PyL (5 mg/mL ascorbic acid in both FPV and delivery solution) and Lot 220817PyL (10 mg/mL ascorbic acid in just the FPV diluted to 5 mg/mL with addition of delivery solution) are similar 97.9% versus 97.8% and 0.52% versus 0.6%, respectively, and indicate delivery of the product from the HLB using pH 6.0 ascorbic acid (5 mg/mL) does not offer additional stabilization, Table 22.

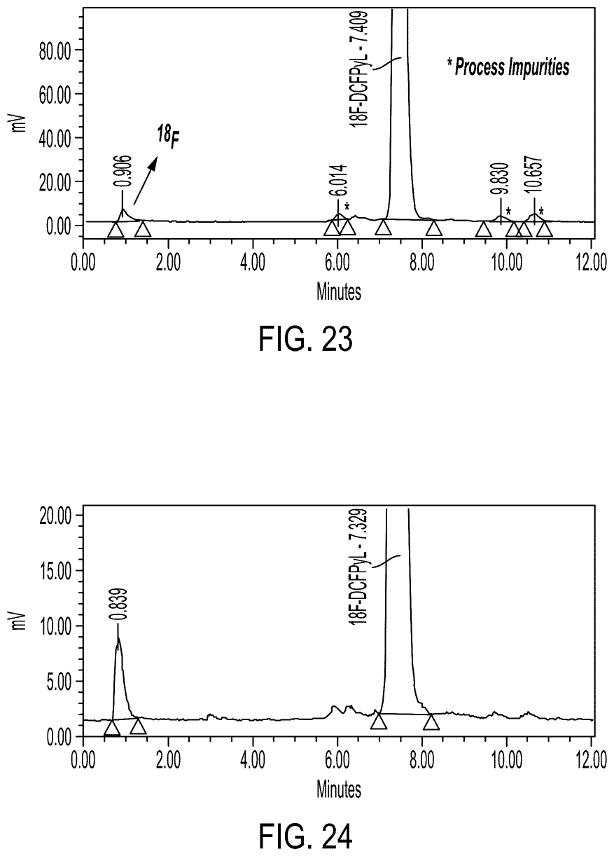

Lot 220908PyL (HCI) was produced to determine if the impurities at approximate RT's of 6.0, 9.8 and 10.6 were present in the peak cut from the semi-prep HPLC purification. The peak cut was delivered into the collection vial containing 35 mL SWFI. The contents of the collection vial were immediately delivered to the FPV containing 16 mL of 0.9% saline and the contents analyzed immediately without further processing. This provided for assessment of whether the impurities observed are process impurities. The peaks at RT's of 6.0, 9.8, and 10.6 minutes were present at RC impurities of 2.13, 1.42, and 0.72%, respectively, and are consistent with levels observed in lots produced with ascorbic acid in the FPV, Table 22. This further supports that the peaks at RT's 6.0, 9.8 and 10.6 are process impurities. The radiochemical chromatogram is provided in FIG. 23.

### Addition of Ascorbic Acid to the Collection Vial (Position I, FIG. 13)

Further studies were designed to determine if the initial RCP for [¹⁸F]DCFPyL could be increased through the stabilization of the purified product prior to the solvent exchange step on the HLB cartridge. Ascorbic acid was added to the collection vial (Position I, FIG. 13) and/or to the “delivery solution vial” (Position F, FIG. 13) as indicated in Table 23. A listing of solution compositions for these studies is provide in Table 24.

Lot 220909Pyl (HCI) was produced using pH 6.0 ascorbic acid loaded into the collection vial and the FPV, respectively. The concentration of ascorbic acid in the FPV after delivery of the product from the HLB cartridge was 5.6 mg/mL. The pH of the product in the collection vial after delivery of the peak cut was 4.7. The radiochemical concentration in the FPV was 89 mCi/mL, which was significantly lower than the >120 mCi/mL expected. The initial RCP was 99%, and remained >97% at 4 hours, Table 25. While this study demonstrated good stability of [¹⁸F]DCFPyL, the radioactive concentration was not high enough to support an assessment of stability at high radioactive concentrations. The process impurities were below reportable levels (<0.3%) at T=4 hours, FIG. 24. The results from this study in conjunction with the results from the analysis of the peak cut analyzed without further processing (Lot 220908PyL), indicate the impurities are degradation products and not process impurities since they can be decreased by stabilizing the product with ascorbate prior to solvent exchange. High levels of radioactivity were detected by the AIO waste line radiodetector during the solvent exchange indicating the product was not well retained on the HLB cartridge. It is hypothesized that [¹⁸F]DCFPyL retention on the HLB cartridge decreases as the pH increases, consistent with increased ionization of [¹⁸F]DCFPyL as the pH is increased.

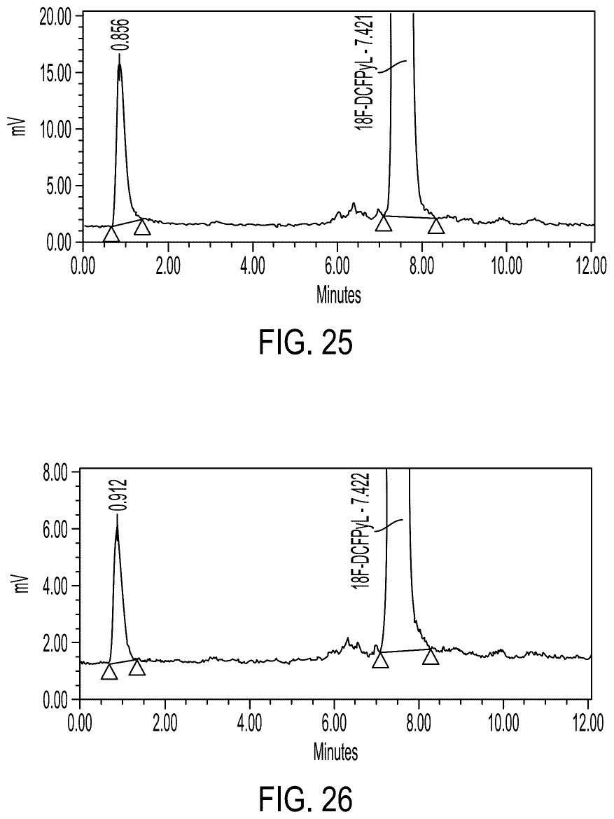

Lot 220914PyL (HCI) was produced with 5.0 mg/mL, pH 6 ascorbic acid in the delivery solution vial and in the FPV. The collection vial contained 5.0 mg/mL, pH 2 ascorbic acid to test whether the pH of the sample loaded onto the HLB cartridge impacts [¹⁸F]DCFPyL retention. The radioactive concentration of the product was 159 mCi/mL, the RCP was 99.0% and 97.6% at T=0 and T=4 hours, respectively. The radiochemical impurities were not at reportable levels, Table 25. The T=4 hour radiochromatogram is provided in FIG. 25. The RCP results are similar to the results for product produced without adding the ascorbic acid to the saline vial (Lot 220909PyL; 99.6% and 98.8% at T=0 and T=4 hours, respectively, Table 25). Lowering the collection vial pH to 2 resulted in retention of the product with virtually no [¹⁸F]DCFPyL being observed in the HLB cartridge washes (based on low radioactivity being detected by the AIO waste line radiodetector).

Lot 220927 (HCI) was prepared using 10.0 mg/mL, pH 6.0 ascorbic acid in the FPV vial and 5.0 mg/mL, pH 2 ascorbic acid loaded into the collection vial. The radiochemical concentration of the lot was 127 mCi, and the RCP was 99.0% and 98.1% at T=0 and T=10 hours, respectively, Table 25. The radiochemical impurities were not at reportable levels though they were near the limit (0.3%); the radiochromatogram is provided in FIG. 26. The results for this lot are similar to the results for Lot 220914PyL (99.0 and 98.6 at T=0 and T=4 hours, respectively [no ascorbic acid in the delivery vial], Table 26), indicating the presence of ascorbic acid in the saline vial does not improve stability relative to only adding it to the collection vial.

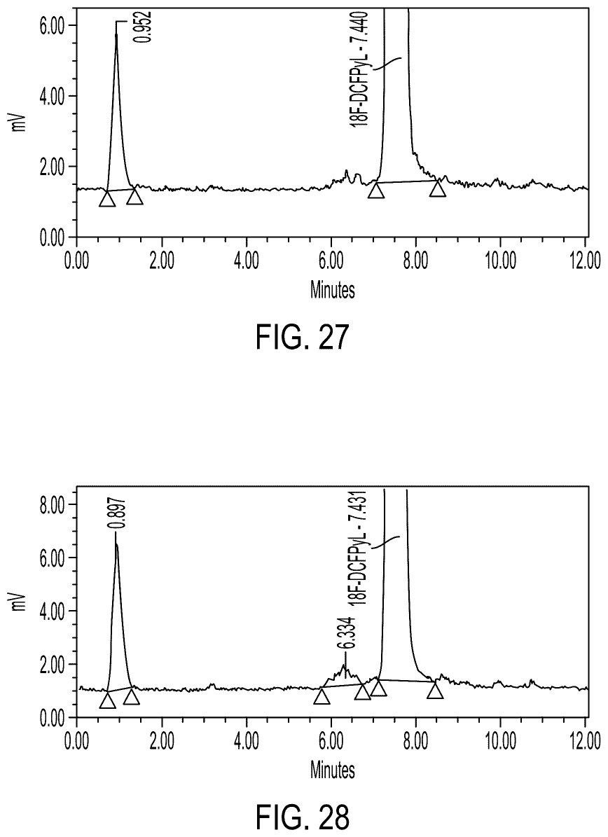

Lot 220928PyL (HCI) was prepared 10 mg/mL ascorbic acid, pH 6.0 in the delivery vial, 5 mg/mL, pH 2.0 ascorbic acid in SWFI in the collection vial and saline in the FPV. The radiochemical concentration was 138 mCi/mL, the RCP was 99.7%, 98.3% and 97.8% at T=0, T=4 and T=10 hours, reproducing the results of Lot 220927Pyl, 99.0%, 98.0% and 98.0%, at T=0, T=4 and T=10 hours, Table 25. There were no reportable radiochemical impurities present; the radiochemical chromatogram is provided in FIG. 27. Addition of ascorbic acid to the saline vial offers no advantage over putting the ascorbic acid in the saline vial.

TABLE 23
Position of ascorbic acid additions for initial collection vial ascorbic acid studies

| | | Lot | Lot | Lot | Lot |
|---|---|---|---|---|---|
| Vial | | 220909PyL | 220914PyL | 220927PyL | 220928PyL |
| Collection | Ascorbic | + | + | + | + |
| vial | Acid | | | | |
| (Position I, | Saline | − | − | − | − |
| FIG. 13) | SWFI | + | + | + | + |
| Delivery | Ascorbic | − | + | − | + |
| solution vial | Acid | | | | |
| (Position F, | Saline | + | + | + | + |
| FIG. 13) | SWFI | − | − | − | − |
| FPV | Ascorbic | + | + | + | − |
| | Acid | | | | |
| | Saline | + | + | + | + |
| | SWFI | − | − | − | − |

TABLE 24
Studies with ascorbic acid added to collection vial (Position I, FIG. 13)

| | Lot | Lot | Lot | Lot |
|---|---|---|---|---|
| Vial | 220909PyL | 220914PyL | 220927PyL | 220928PyL |
| Collection | 35 mL of 5.0 | 35 mL of 5.0 | 35 mL of 5.0 | 35 mL of 5.0 |
| vial | mg/mL pH 6.0 | mg/mL pH 2.0 | mg/mL pH 2.0 | mg/mL pH 2.0 |
| (Position I, | ascorbic acid | ascorbic acid | ascorbic acid | ascorbic acid |
| FIG. 13) | in SWFI | in SWFI | in SWFI | in SWFI |
| Delivery | Fill: 20 mL of 0.9% | Fill: 20 mL of 5.0 | Fill: 20 mL of 0.9% | Fill: 20 mL of 10.0 |
| solution vial | sodium chloride, | mg/mL pH 6.0 | sodium chloride, USP | mg/mL pH 6.0 |
| (Position F, | USP | ascorbic acid in | Delivery: 16 mL | ascorbic acid in 0.9% |
| FIG. 13) | Delivery: 16 mL | 0.9% sodium | to FPV | sodium chloride, USP |
| | to FPV | chloride, USP | | Delivery: 16 mL |
| | | Delivery: 16 mL | | to FPV |
| | | to FPV | | |
| FPV | 20 mL of 10.0 | 20 mL of 5.0 | 20 mL of 10.0 | 20 mL 0.9% |
| | mg/mL ascorbic acid, | mg/mL ascorbic acid, | mg/mL ascorbic acid, | sodium chloride, |
| | pH 6.0 in 0.9% | pH 6.0 in 0.9% | pH 6.0 in 0.9% | USP |
| | sodium chloride, | sodium chloride, | sodium chloride, | |
| | USP | USP | USP | |
| Ascorbic acid | 5.6 mg/mL^{a} | 5.0 mg/mL^{b} | 5.6 mg/mL^{a} | 4.4 mg/mL^{c} |
| concentration | | | | |
| after delivery | | | | |
| of all | | | | |
| components | | | | |

^{a}16 mL 0.9% sodium chloride, USP, plus 20 mL of 10 mg/mL ascorbic acid = (20 mL × 10 mg/mL)/36 mL = 5.6 mg/mL ascorbic acid
^{b}16 mL of 5.0 mg/mL ascorbic acid in 0.9% sodium chloride, USP, plus 20 mL of 5 mg/mL ascorbic acid in 0.9% sodium chloride, USP = [(16 mL × 5 mg/mL) + (20 mL × 5 mg/mL)/36 mL = 5.0 mg/mL ascorbic acid
^{c}16 mL of 10.0 mg/mL ascorbic acid in 0.9% sodium chloride, USP, plus 20 mL of 0.9% sodium chloride, USP = [(16 mL × 5 mg/mL) + (20 mL × 5 mg/mL)/36 mL = 4.4 mg/mL ascorbic acid

TABLE 25
Addition of ascorbic acid to the collection vial

| | Lot 220909PyL | Lot 220914PyL | Lot 220927PyL | Lot 220928PyL |
|---|---|---|---|---|
| Starting activity | 13.7 Ci | 13.9 | 13.7 | 13.5 |
| mCi/mL | 89 | 159 | 127 | 138 |
| Collection Vial | 5.0 mg/mL pH 6.0 | 5.0 mg/mL pH 2.0 | 5.0 mg/mL pH 2.0 | 5.0 mg/mL pH 2.0 |
| | ascorbic acid in SWFI | ascorbic acid in SWFI | ascorbic acid in SWFI | ascorbic acid in SWFI |
| Delivery | 0.9% sodium | 5.0 mg/mL pH 6.0 | 0.9% sodium | 10.0 mg/mL pH 6.0 |
| Solution Vial | chloride, USP | ascorbic acid in 0.9% | chloride, USP | ascorbic acid in 0.9% |
| | | sodium chloride, USP | | sodium chloride, USP |
| FPV | 10.0 mg/mL ascorbic | 5.0 mg/mL ascorbic | 10.0 mg/mL ascorbic | 0.9% sodium |
| | acid, pH 6.0 in 0.9% | acid, pH 6.0 in 0.9% | acid, pH 6.0 in 0.9% | chloride, USP |
| | sodium chloride, USP | sodium chloride, USP | sodium chloride, USP | |
| Product | 5.6 mg/mL | 5.0 mg/mL | 5.6 mg/mL | 4.4 mg/mL |
| ascorbic acid | | | | |
| concentration | | | | |

| | T = 0 | T = 4 | T = 10 | T = 0 | T = 4 | T = 10 | T = 0 | T = 4 | T = 10 | T = 0 | T = 4 | T = 10 |
|---|---|---|---|---|---|---|---|---|---|---|---|---|
| RCP | 99.6 | 98.8 | nt^{a} | 99.0 | 97.6 | nt | 99.0 | 98.0 | 98.0 | 99.7 | 98.3 | 97.8 |
| 18F | 0.39 | 1.17 | nt | 0.97 | 2.00 | nt | 0.59 | 1.70 | 1.95 | 0.32 | 1.70 | 2.09 |
| RT 6.0 | <LLQ^{b} | <LLQ | nt | <LLQ | <LLQ | nt | 0.31 | 0.35 | <LLQ | <LLQ | <LLQ | <LLQ |
| RT 9.8 | <LLQ | <LLQ | nt | <LLQ | <LLQ | nt | <LLQ | <LLQ | <LLQ | <LLQ | <LLQ | <LLQ |
| RT 10.6 | <LLQ | <LLQ | nt | <LLQ | <LLQ | nt | <LLQ | <LLQ | <LLQ | <LLQ | <LLQ | <LLQ |

nt = not tested
LLQ = Lower than Limit of Quantitation

TABLE 26
Addition of ascorbic acid to the collection vial

| | Lot 220909PyL | Lot 220914PyL | Lot 220927PyL | Lot 220928PyL |
|---|---|---|---|---|
| Starting activity | 13.7 Ci | 13.9 | 13.7 | 13.5 |
| mCi/mL | 89 | 159 | 127 | 138 |
| Collection Vial | 5.0 mg/mL pH 6.0 | 5.0 mg/mL pH 2.0 | 5.0 mg/mL pH 2.0 | 5.0 mg/mL pH 2.0 |
| | ascorbic acid in SWFI | ascorbic acid in SWFI | ascorbic acid in SWFI | ascorbic acid in SWFI |
| Delivery | 0.9% sodium chloride, | 5.0 mg/mL pH 6.0 | 0.9% sodium chloride, | 10.0 mg/mL pH 6.0 |
| Solution Vial | USP | ascorbic acid in 0.9% | USP | ascorbic acid in 0.9% |
| | | sodium chloride, USP | | sodium chloride, USP |
| FPV | 10.0 mg/mL ascorbic | 5.0 mg/mL ascorbic | 10.0 mg/mL ascorbic | 0.9% sodium chloride, |
| | acid, pH 6.0 in 0.9% | acid, pH 6.0 in 0.9% | acid, pH 6.0 in 0.9% | USP |
| | sodium chloride, USP | sodium chloride, USP | sodium chloride, USP | |
| Product | 5.6 mg/mL | 5.0 mg/mL | 5.6 mg/mL | 4.4 mg/mL |
| ascorbic acid | | | | |
| concentration | | | | |

| | T = 0 | T = 4 | T = 10 | T = 0 | T = 4 | T = 10 | T = 0 | T = 4 | T = 10 | T = 0 | T = 4 | T = 10 |
|---|---|---|---|---|---|---|---|---|---|---|---|---|
| RCP | 99.6 | 98.8 | nt^{a} | 99.0 | 97.6 | nt^{a} | 99.0 | 98.0 | 98.0 | 99.7 | 98.3 | 97.8 |
| 18F | 0.39 | 1.17 | nt^{a} | 0.97 | 2.00 | nt^{a} | 0.59 | 1.70 | 1.95 | 0.32 | 1.70 | 2.09 |
| RT 6.0 | <LLQ^{b} | <LLQ^{b} | nt^{a} | <LLQ^{b} | <LLQ^{b} | nt^{a} | 0.31 | 0.35 | <LLQ^{b} | <LLQ^{b} | <LLQ^{b} | <LLQ^{b} |
| RT 9.8 | <LLQ^{b} | <LLQ^{b} | nt^{a} | <LLQ^{b} | <LLQ^{b} | nt^{a} | <LLQ^{b} | <LLQ^{b} | <LLQ^{b} | <LLQ^{b} | <LLQ^{b} | <LLQ^{b} |
| RT 10.6 | <LLQ^{b} | <LLQ^{b} | nt^{a} | <LLQ^{b} | <LLQ^{b} | nt^{a} | <LLQ^{b} | <LLQ^{b} | <LLQ^{b} | <LLQ^{b} | <LLQ^{b} | <LLQ^{b} |

^{a}nt = not tested
^{b}LLQ = Lower than Limit of Quantitation

### Effect of FPV pH and Ascorbic Acid Concentration on Stability of Formulations

Based on the studies that showed a pH drift to approximately pH 6.8 at T=10 hours, a pH target for the formulation of 5.5 was chosen for the ascorbic acid formulation. Production included loading the collection vial with 35 mL of 10 mg/mL, pH 2 ascorbic acid in 0.9% sodium chloride, USP, and preloading 20 mL of 10 mg/mL, pH 5.5 ascorbic acid in 0.9% sodium chloride, USP, into the FPV. The resulting ascorbic acid concentration in the product is 5.6 mg/mL after delivery of the purified product from the HLB solvent exchange cartridge to the FPV.

The impact of FPV pH on the stability of [¹⁸F]DCFPyL was studied at the target pH and the pH limits of 4.5 and 7.0. Lots were prepared at pH 4.5, 5.5 and 7.0, and samples were analyzed at 0, 2, 4, 6, and 10 hours for RCP and pH. Table 27 provides a summary of the composition of the delivery solution vial, the collection vial and the FPV.

Lot 221025Pyl was prepared with 10 mg/mL, pH 2.0 ascorbic acid in the collection vial and 10.0 mg/mL, pH 5.5 ascorbic acid in the FPV. The radiochemical concentration of the product was 133 mCi/mL. The RCP was 98.9% at 0 hours and 97.4% at 10 hours, Table 28. There was one radiochemical impurity present at ˜0.5% through 4 hours that was not present after 4 hours. No other impurities were present at a reportable level; the T=10 hour chromatogram is provided in FIG. 28.

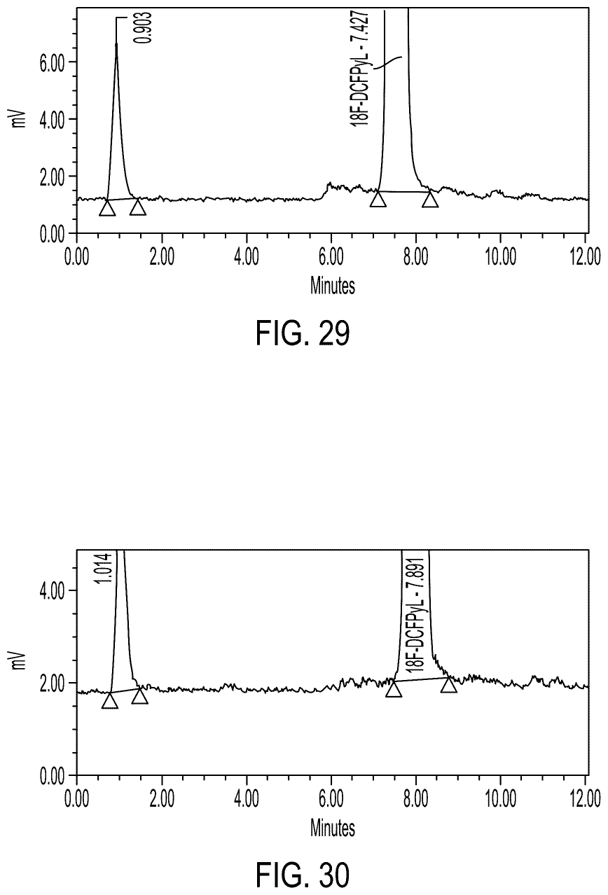

Lot 221026Pyl was prepared with 10 mg/mL, pH 2.0 ascorbic acid in the collection vial and 10.0 mg/mL, pH 4.5 ascorbic acid in the FPV. The radiochemical concentration of the product was 133 mCi/mL. The RCP was 99.4% at 0 hours and 98.0% at 10 hours, Table 28. There were no reportable radiochemical impurities present throughout the study. The T=10 hour chromatogram is provided in FIG. 29.

Lot 230124Pyl was prepared with 10 mg/mL, pH 2.0 ascorbic acid in the collection vial and 10.0 mg/mL, pH 7.5 ascorbic acid in the FPV The radiochemical concentration of the product was 162 mCi/mL. The RCP was 99.2% at 0 hours and 97.4% at 10 hours, Table 28. There were no reportable radiochemical impurities present throughout the study. The T=10 hour chromatogram is provided in FIG. 30.

TABLE 27
Studies of [18F]DCFPyL product at different pHs

| Vial | Lot 221026PyL | Lot 221025PyL | Lot 230124PyL |
|---|---|---|---|
| Collection vial | 35 mL of 10.0 mg/mL pH 2.0 | 35 mL of 10.0 mg/mL pH 2.0 | 35 mL of 10.0 mg/mL pH 2.0 |
| (Position I, | ascorbic acid in 0.9% | ascorbic acid in 0.9% | ascorbic acid in 0.9% |
| FIG. 13) | sodium chloride, USP | sodium chloride, USP | sodium chloride, USP |
| Delivery | Fill: 20 mL of 0.9% | Fill: 20 mL of 0.9% | Fill: 20 mL of 0.9% |
| solution vial | sodium chloride, USP | sodium chloride, USP | sodium chloride, USP |
| (Position F, | Delivery: 16 mL to FPV | Delivery: 16 mL to FPV | Delivery: 16 mL to FPV |
| FIG. 13) | | | |
| FPV | 20 mL of 10.0 mg/mL | 20 mL of 10.0 mg/mL | 20 mL of 10.0 mg/mL |
| | ascorbic acid, | ascorbic acid, | ascorbic acid, |
| | pH 4.5 in 0.9% | pH 5.5 in 0.9% | pH 7.0 in 0.9% |
| | sodium chloride, USP | sodium chloride, USP | sodium chloride, USP |
| Product | 5.6 mg/mL, pH 4.5 | 5.6 mg/mL, pH 5.5 | 5.6 mg/mL, pH 7.0 |
| ascorbic acid | | | |
| concentration^{a} | | | |
| and pH | | | |

^{a}16 mL 0.9% sodium chloride, USP, plus 20 mL of 10 mg/mL ascorbic acid = (20 mL × 10 mg/mL)/36 mL = 56 mg/mL ascorbic acid

TABLE 28
Data for [18F]DCFPyL produced at different pHs

| | Lot 221026PyL | Lot 221025PyL | Lot 230124PyL |
|---|---|---|---|
| pH | 4.5 | 5.5 | 7.0 |
| Starting activity | 14.7 | 14.7 | 16.5 |
| mCi/mL | 133 | 133 | 162 |
| Collection Vial | 10.0 mg/mL pH 2.0 | 10.0 mg/mL pH 2.0 | 10.0 mg/mL pH 2.0 |
| | ascorbic acid in 0.9% | ascorbic acid in 0.9% | ascorbic acid in 0.9% |
| | sodium chloride, USP | sodium chloride, USP | sodium chloride, USP |
| Delivery | 0.9% sodium | 0.9% sodium | 0.9% sodium |
| Solution Vial | chloride, USP | chloride, USP | chloride, USP |
| FPV | 10.0 mg/mL ascorbic | 5.0 mg/mL ascorbic | 10.0 mg/mL ascorbic |
| | acid, pH 4.5 in 0.9% | acid, pH 5.5 in 0.9% | acid, pH 7.0 in 0.9% |
| | sodium chloride, USP | sodium chloride, USP | sodium chloride, USP |
| Product | 5.6 mg/mL | 5.6 mg/mL | 5.6 mg/mL |
| ascorbic acid | | | |
| concentration | | | |

| Timepoint (hr) | T = 0 | T = 2 | T = 4 | T = 6 | T = 10 | T = 0 | T = 2 | T = 4 |
|---|---|---|---|---|---|---|---|---|
| RCP | 99.4 | 98.8 | 98.3 | 98.1 | 98.0 | 98.7 | 98.2 | 98.1 |
| 18F | 0.61 | 1.22 | 1.68 | 1.92 | 2.04 | 0.71 | 1.33 | 1.89 |
| RT 6.0 | <LLQ^{a} | <LLQ^{a} | <LLQ^{a} | <LLQ^{a} | <LLQ^{a} | 0.42 | 0.52 | <LLQ^{a} |
| RT 9.8 | <LLQ^{a} | <LLQ^{a} | <LLQ^{a} | <LLQ^{a} | <LLQ^{a} | <LLQ^{a} | <LLQ^{a} | <LLQ^{a} |
| RT 10.6 | <LLQ^{a} | <LLQ^{a} | <LLQ^{a} | <LLQ^{a} | <LLQ^{a} | <LLQ^{a} | <LLQ^{a} | <LLQ^{a} |

| Timepoint (hr) | T = 6 | T = 10 | T = 0 | T = 2 | T = 4 | T = 6 | T = 10 |
|---|---|---|---|---|---|---|---|
| RCP | 97.9 | 97.4 | 99.2 | 98.2 | 97.8 | 97.3 | 97.4 |
| 18F | 2.10 | 2.08 | 0.83 | 1.84 | 2.24 | 2.71 | 2.65 |
| RT 6.0 | <LLQ^{a} | 0.53^{a} | <LLQ^{a} | <LLQ^{a} | <LLQ^{a} | <LLQ^{a} | <LLQ^{a} |
| RT 9.8 | <LLQ^{a} | <LLQ^{a} | <LLQ^{a} | <LLQ^{a} | <LLQ^{a} | <LLQ^{a} | <LLQ^{a} |
| RT 10.6 | <LLQ^{a} | <LLQ^{a} | <LLQ^{a} | <LLQ^{a} | <LLQ^{a} | <LLQ^{a} | <LLQ^{a} |

^{a}LLQ = Lower than Limit of Quantitation

[¹⁸F]DCFPyL produced at the extremes of the pH specification (4.5 and 7.0) for radiochemical concentrations up to 165 mCi/mL is projected to meet the RCP and chemical impurity product specifications for related substances over 10 hours post EOS. The lowest actual RCP value was 97.4%, which was also at the highest pH (7.5) and highest radioactive concentration (162 mCi/mL). This study supports the use of a 5.5 mg/mL, pH 5.5 ascorbic acid formulation in an embodiment and provides confirmation that product at or near the pH specification acceptance limits of 4.0 to 7.0 will meet the RCP specification of ≥96% at concentrations up to 165 mCi/mL.

The impact of ascorbic acid on the stability of [¹⁸F]DCFPyL was studied at the target ascorbic acid level and at the specification limits of 3.0 and 7.0 mg/mL. Lots were prepared, and samples were analyzed at 0, 2, 4, 6, and 10 hours for RCP and pH. Table 29 provides a summary of the composition of the delivery solution vial, the collection vial and the FPV.

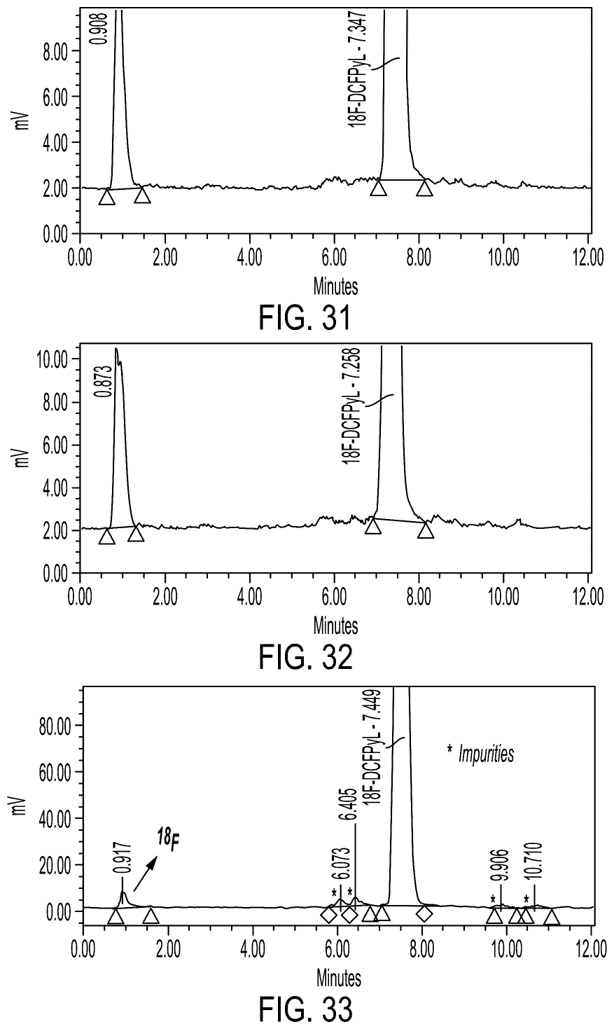

Lot 230130PyL was prepared with 10 mg/mL, pH 2.0 ascorbic acid in the collection vial and 3.3 mg/mL, pH 5.5 ascorbic acid in the FPV. The radiochemical concentration of the product was 150 mCi/mL. The RCP was 99.0% at 0 hours and 96.7% at 10 hours, Table 30. No other radiochemical impurities were present at a reportable level; the T=10 hour chromatogram is provided in FIG. 31.

Lot 221026PyL was prepared with 10 mg/mL, pH 2.0 ascorbic acid in the collection vial and 10.0 mg/mL, pH 5.5 ascorbic acid in the FPV. The radiochemical concentration of the product was 133 mCi/mL. The RCP was 99.4% at 0 hours and 98.0% at 10 hours, Table 30. There were no reportable radiochemical impurities present throughout the study. The T=10 hour chromatogram is provided in FIG. 29.

Lot 230131PyL was prepared with 10 mg/mL, pH 2.0 ascorbic acid in the collection vial and 14.0 mg/mL, pH 5.5 ascorbic acid in the FPV. The radiochemical concentration of the product was 165 mCi/mL. The RCP was 99.0% at 0 hours and 97.3% at 10 hours, Table 30. There were no reportable radiochemical impurities present throughout the study. The T=10 hour chromatogram is provided in FIG. 32.

TABLE 29
Studies of [18F]DCFPyL product at different ascorbic acid levels

| Vial | Lot 230130PyL | Lot 221025PyL | Lot 230131PyL |
|---|---|---|---|
| Collection vial | 35 mL of 10.0 mg/mL pH 2.0 | 35 mL of 10.0 mg/mL pH 2.0 | 35 mL of 10.0 mg/mL pH 2.0 |
| (Position I, | ascorbic acid in 0.9% | ascorbic acid in 0.9% | ascorbic acid in 0.9% |
| FIG. 13) | sodium chloride, USP | sodium chloride, USP | sodium chloride, USP |
| Delivery | Fill: 20 mL of 0.9% | Fill: 20 mL of 0.9% | Fill: 20 mL of 0.9% |
| solution vial | sodium chloride, USP | sodium chloride, USP | sodium chloride, USP |
| (Position F, | Delivery: 16 mL to FPV | Delivery: 16 mL to FPV | Delivery: 16 mL to FPV |
| FIG. 13) | | | |
| FPV | 20 mL of 6.0 mg/mL | 20 mL of 10.0 mg/mL | 20 mL of 14.0 mg/mL |
| | ascorbic acid, | ascorbic acid, | ascorbic acid, |
| | pH 4.5 in 0.9% | pH 5.5 in 0.9% | pH 7.0 in 0.9% |
| | sodium chloride, USP | sodium chloride, USP | sodium chloride, USP |
| Product | 3.3 mg/mL, pH 5.5^{a} | 5.6 mg/mL, pH 5.5^{b} | 7.8 mg/mL, pH 5.5^{c} |
| ascorbic acid | | | |
| concentration | | | |
| and pH | | | |

^{a}16 mL 0.9% sodium chloride, USP, plus 20 mL of 6 mg/mL ascorbic acid = (20 mL × 10 mg/mL)/36 mL = 3.3 mg/mL ascorbic acid
^{b}16 mL 0.9% sodium chloride, USP, plus 20 mL of 10 mg/mL ascorbic acid = (20 mL × 10 mg/mL)/36 mL = 5.6 mg/mL ascorbic acid
^{5.5c}16 mL 0.9% sodium chloride, USP, plus 20 mL of 14 mg/mL ascorbic acid = (20 mL × 10 mg/mL)/36 mL = 7.8 mg/mL ascorbic acid

TABLE 30
Data for [18F]DCFPyL produced at different ascorbic acid concentrations

| | Lot 230130PyL | Lot 221025PyL | Lot 230131PyL |
|---|---|---|---|
| pH | 5.5 | 5.5 | 5.5 |
| Starting | 15.6 | 14.7 | 16.4 |
| activity | | | |
| mCi/mL | 150 | 133 | 165 |
| Collection | 10.0 mg/mL pH 2.0 ascorbic acid in | 10.0 mg/mL pH 2.0 ascorbic acid in | 10.0 mg/mL pH 2.0 ascorbic acid in |
| Vial | 0.9% sodium chloride, USP | 0.9% sodium chloride, USP | 0.9% sodium chloride, USP |
| Delivery | 0.9% sodium chloride, USP | 0.9% sodium chloride, USP | 0.9% sodium chloride, USP |
| Solution | | | |
| Vial | | | |
| FPV | 6.0 mg/mL ascorbic acid, pH 4.5 in | 10.0 mg/mL ascorbic acid, pH 5.5 in | 14.0 mg/mL ascorbic acid, pH 7.0 in |
| | 0.9% sodium chloride, USP | 0.9% sodium chloride, USP | 0.9% sodium chloride, USP |
| Product | 3.3 mg/mL | 5.6 mg/mL | 7.8 mg/mL |
| ascorbic acid | | | |
| concentration | | | |

| Timepoint (hr) | T = 0 | T = 2 | T = 4 | T = 6 | T = 10 | T = 0 | T = 2 | T = 4 |
|---|---|---|---|---|---|---|---|---|
| RCP | 99.0 | 98.0 | 97.4 | 96.9 | 96.7 | 98.7 | 98.2 | 98.1 |
| 18F | 1.05 | 2.02 | 2.58 | 3.11 | 3.35 | 0.71 | 1.33 | 1.89 |
| RT 6.0 | <LLQ^{a} | <LLQ^{a} | <LLQ^{a} | <LLQ^{a} | <LLQ^{a} | 0.42 | 0.52 | <LLQ^{a} |
| RT 9.8 | <LLQ^{a} | <LLQ^{a} | <LLQ^{a} | <LLQ^{a} | <LLQ^{a} | <LLQ^{a} | <LLQ^{a} | <LLQ^{a} |
| RT 10.6 | <LLQ^{a} | <LLQ^{a} | <LLQ^{a} | <LLQ^{a} | <LLQ^{a} | <LLQ^{a} | <LLQ^{a} | <LLQ^{a} |

| Timepoint (hr) | T = 6 | T = 10 | T = 0 | T = 2 | T = 4 | T = 6 | T = 10 |
|---|---|---|---|---|---|---|---|
| RCP | 97.9 | 97.9 | 99.0 | 98.2 | 97.7 | 97.6 | 97.3 |
| 18F | 2.10 | 2.09 | 0.94 | 1.80 | 2.33 | 2.39 | 2.75 |
| RT 6.0 | <LLQ^{a} | <LLQ^{a} | <LLQ^{a} | <LLQ^{a} | <LLQ^{a} | <LLQ^{a} | <LLQ^{a} |
| RT 9.8 | <LLQ^{a} | <LLQ^{a} | <LLQ^{a} | <LLQ^{a} | <LLQ^{a} | <LLQ^{a} | <LLQ^{a} |
| RT 10.6 | <LLQ^{a} | <LLQ^{a} | <LLQ^{a} | <LLQ^{a} | <LLQ^{a} | <LLQ^{a} | <LLQ^{a} |

^{a}LLQ = Lower than Limit of Quantitation

[¹⁸F]DCFPyL produced at ascorbic acid target and specification levels (3.0, 5.0 and 7.0 mg/mL) for radiochemical concentrations up to 165 mCi/mL is projected to meet the RCP and chemical impurity product specifications for related substances over 10 hours post EOS. The lowest actual RCP value was 96.7% for the lot having a 3.0 mg/mL ascorbic acid concentration and a radioactive concentration of 150 mCi/mL. This study supports 5.5 mg/mL, pH 5.5 ascorbic acid formulation in an embodiment and provides confirmation that product at or near ascorbic acid specification acceptance limits of 3.0 to 7.0 mg/mL will meet the RCP specification of ≥95% at concentrations up to 165 mCi/mL.

### Effect of pH on [¹⁸F]DCFPyL Retention on the HLB Cartridge

The goal of this study was to characterize the impact of pH on the retention of [¹⁸F]DCFPyL on the HLB solvent exchange cartridge. Three separate 10 mg/mL ascorbic acid solutions were prepared. The pH of one solution was adjusted to 2 and a second to 4 by adding 1N HCl. A third solution was used without adjusting its native pH of 7.2. Using a BD syringe, 10 mL of each solution was spiked with [¹⁹F]DCFPyL (50 μg) by adding 0.5 mL of a 100 μg/mL stock solution. Each spiked solution was loaded onto a HLB cartridge at a rate of approximately 2 mL/min. The eluent from each HLB cartridge was individually collected, and is identified as the “load solution.” Each HLB cartridge was then rinsed with 10 mL water at a rate of approximately 2 mL/min. The eluent from each HLB cartridge was individually collected and identified as the “wash solution.” Each HLB cartridge was then individually eluted with 1 mL of ethanol, the samples collected and are identified as the “product sample.” The load solution and wash solution samples were analyzed using a drug product chemical impurity assay after a 1 to 10 mL dilution with saline, while the ethanolic product samples were analyzed following a 0.5 to 10 dilution with saline.

The [¹⁹F]DCFPyL concentration of the load and recovery samples varied as a function of pH. At pH of 7.2 the ¹⁹F-DCFPYL was not retained by the HLB cartridge and was recovered in the load sample. A small amount of [¹⁹F]DCFPyL was recovered in the wash samples (3.8 μg/mL), while there was no recovery in the product samples. In the pH 4 samples a portion of the [¹⁹F]DCFPyL was recovered in the load sample (approximately 31%<1 μg) and a larger amount in the product sample (approximately 68% of the total amount recovered from all samples). At pH 2, [¹⁹F]DCFPyL was not detected in the load or wash samples. The [¹⁹F]DCFPyL was recovered in the product solution. A pH 2 HLB cartridge was eluted with a second volume of ethanol with 98% recovery. An additional recovery of [¹⁹F]DCFPyL was observed (approximately 25% of the amount recovered in the first elution).

The recovers were based on the concentration in the samples and the volumes of the solutions applied. Loss of volume in dead space or adsorption of [19F]DCFPyL on surfaces may explain overall recoveries accounting for less than 100%. Nonetheless, it is clear retention is optimized at lower pH (e.g., pH 2). These studies indicate the pH of the collection vial solution (the solution loaded onto the HLB solvent exchange cartridge) can be set to pH 2 in an embodiment.

TABLE 31
Recoveries from the solvent exchange HLB cartridge
for load, rinse and product samples

| Sample | μg of ¹⁹F-DCFPyL recovered (%) |
|---|---|
| pH 7 Control | 43.759 |
| HLB pH 7 Load | 38.239 (87.4%) |
| HLB Saline pH 7 Rinse | Not Detected (0.0%) |
| HLB pH 7 Product | Not Detected (0.0%) |
| Combined recovery for | 87.4% |
| pH 7 samples | |
| pH 4 Control | 47.665 |
| HLB pH 4 Load | 10.504 (22.0%) |
| HLB pH 4 Rinse | Not Detected (0.0%) |
| HLB pH 4 Product | 27.989 (57.7%) |
| Combined recovery for | 80.8% |
| pH 4 samples | |
| pH 2 Control | 50.976 |
| HLB pH 2 Load | Not Detected (0.0%) |
| HLB pH 2 Rinse | Not Detected (0.0%) |
| HLB pH 2 Product, first | 35.423 (69.5%) |
| Ethanol elution | |
| HLB pH 2 Product, second | 10.946 (21.5%) |
| Ethanol elution | |
| HLB pH 2 Product, third | 3.629 (7.1%) |
| Ethanol elution | |
| Combined recovery for | 69.5% |
| pH 2 one Ethanol elution | |
| Combined recovery for | 91.0% |
| pH 2 two Ethanol elutions | |
| Combined recovery for | 98.1% |
| pH 2 three Ethanol elutions | |

### Example 13. Formulations of [¹⁸F]DCFPyL

A composition of PYLARIFY is provided in Table 33. The formulation is 0.9% Sodium Chloride, USP, with up to 7.89% ethanol, w/v (typical lots contain approximately 3% ethanol). A process scheme is provided in FIG. 13. A schematic of the Trasis ALLinONE (AIO) synthesis module with reagents and reagent positions indicated is provided in FIG. 14. This product has an upper limit of 80 mCi/mL radioactive concentration at end of synthesis (EOS). The major radioactive degradation product observed is free ¹⁸F and is formed through radiolysis of ¹⁸F-DCFPyL. The rapid commercial uptake of PYLARIFY has resulted in the need for multiple lots to be produced by some of the commercial PMFs each day.

In order to ensure no restriction of drug supply occurs, a product having a higher radioactive concentration (>120 mCi/mL) at EOS is desirable since it would provide more doses per lot. Studies were undertaken to examine the addition of a radioprotectant to the drug product to minimize possible drug substance radiolysis in order to achieve higher radioactive concentrations. The ability of ascorbic acid to stabilize PYLARIFY at radioactive concentrations>120 mCi/mL was examined. A flow diagram indicating the different places ascorbic acid was introduced in the production process in order to protect the product at points of high radioactivity. Ascorbic acid can be delivered to the final product vial (FPV) as part of the product delivery from the HLB solvent exchange cartridge (ascorbic acid added to the Delivery Solution Vial; position F on the AIO, FIG. 14) and/or by direct introduction into the FPV. In any one of the methods provided herein ascorbic acid is introduced at any one of the concentrations provided herein at such steps. The addition of ascorbic acid to the collection vial used to receive the peak cut from the semi-prep purification also provides a potential path to stabilizing the product prior to the solvent exchange step. In any one of the methods provided herein ascorbic acid is introduced at any one of the concentrations provided herein at such a step.

TABLE 33
PYLARIFY exemplary formulation composition

| Component | Quantity per Dose | Role in Formulation | Quality Standard |
|---|---|---|---|
| ¹⁸F-DCFPyL | 9 mCi @TOA ≤4 μg | Active drug | GMP |
| | chemical mass^{a} | substance | |
| Ethanol 100% | 1 to 7.89% w/v^{b} | Eluent used in | ACS |
| | | drug substance | ISO |
| | | formulation | Eur Ph Reagent |
| 0.9% Sodium Chloride, | 92 to 99% w/v^{c} | Eluent/diluent used | USP |
| USP | | in drug substance | ACS |
| | | formulation | ISO |
| | | | Eur Ph Reagent |

^{a}TOA = Time of Administration
^{b}Depends on batch's specific activity at the end of the synthesis (EOS) and a lag time between EOS and TOA
^{c}The dilution with normal saline post-production is performed to achieve a radiochemical concentration ≤80 mCi/mL at EOS. The degree of dilution varies depending on a starting batch scale (1 Ci to 10 Ci) and process yield.

Exemplary PYLARIFY product specifications are provided in Table 34. A general test plan is provided Table 35 as an example. In embodiments, the test timepoints can be modified.

TABLE 34
PYLARIFY Product Specification

| Test | Acceptance Criteria |
|---|---|
| Appearance | Clear, colorless, particulate-free solution |
| Membrane Filter | ≥50 psi |
| integrity test | |
| pH | 4.5-7.0 |
| Radiochemical Purity | ≥95% |
| Radiochemical Identity^{a} | Calculation within ± 10% |
| Radionuclidic Identity | T_{1/2} = 105-115 min |
| by Half-life | |
| Chemical Purity | Total of unknown impurities ≤1.5 μg/mL |
| Radioactive Concentration | ≥80 mCi/mL at EOS |
| Residual Solvent Analysis | Ethanol: ≤7.89% w/v |
| | Acetonitrile: ≤0.04% w/v |
| Specific Activity at | ≥1000 mCi/μmol at expiration time |
| Expiration^{b} | |
| Ascorbic Acid | Specification can include this test |
| | for the 18F-DCFPyL Ascorbic Acid |
| | formulation |

^{a}Radiochemical Identity = (1 − [RT of hot peak]/[RT of cold peak])*100%
^{b}Expiration time of <10 hours due to specific activity, does not preclude the use of the batch for the purposes of this protocol.

TABLE 312
Test Plan

| | Huntsman Method | |
|---|---|---|
| Test | Reference | Timepoint |
| Appearance | Q015 | 0 |
| Membrane Filter Integrity | Bubble point | 0 |
| | pressure test | |
| pH | Q014 | 0 and 10 hrs |
| Radiochemical Purity | Q529 | 0, 2, 4, 6 and 10 hrs |
| Radiochemical Identity | Q529 | 0 |
| Radionuclidic Identity | Q005 | 0 |
| by Half-life | | |
| Chemical Purity | Q529 | 0, 10 hours |
| Radioactive Concentration | M531 | 0 |
| Residual Solvent Analysis | Q003 | 0 |
| Specific Activity at | Q529 | 0 |
| Expiration | | |
| Ascorbic Acid | Q528 | 0 and 10 hours |

### Preliminary Studies Performed at 18F Starting Activity of Approximately 2 Ci

A feasibility of stabilizing PYLARIFY using ascorbic acid added to: 1) the collection vial used to receive the product peak cut from the semi prep purification step, 2) the preconditioning step for the HLB cartridge used to perform solvent exchange and/or 3) the final product vial was performed. In any one of the methods provided herein ascorbic acid is introduced at any one of the concentrations provided herein at such steps.

The first experiment was done to understand the stability of the product in ethanol as a baseline against which improvements could be measured. It also provided information about the radiochemical impurities that may form at a very high radioactive concentration before the product is exposed to an aqueous medium. The product was collected in a 10 mL GRACE headspace vial which was stoppered and crimped. The final product volume was 1.41 mL, the final product activity at end of synthesis 514 mCi and the final radioactive concentration (RAC) 364.5 mCi/mL for the lot produced. In order to analyze the product at each time point, the stopper was decrimped and aliquots taken by pipette and diluted with saline (at this point the product was no longer in a nitrogen atmosphere but exposed to air). The results of the study are given in

Table 313.

TABLE 313
Forced Radiolysis of ¹⁸F-DCFPyL under High Activity Concentrations

| | T = 0^{a} | T = 1 | T = 2 | T = 3 | T = 4 | T = 5 | T = 6 | T = 7 | T = 8 | T = 9 | T = 10 | T = 10^{b} |
|---|---|---|---|---|---|---|---|---|---|---|---|---|
| ¹⁸F-DCFPyL^{c} | 98.37 | 96.66 | 96.38 | 95.71 | 95.60 | 95.58 | 95.73 | 95.42 | 95.76 | 95.31 | 95.45 | 92.65 |
| Free ¹⁸F^{c} | 1.63 | 3.34 | 3.62 | 4.29 | 4.40 | 4.42 | 4.27 | 4.58 | 4.24 | 4.69 | 4.55 | 7.35 |

^{a}All times are in hours
^{b}TEAF added to sharpen the 18F peak - 44 mg of TEAF plus 1 mL of Product (44 mg/mL)
^{c}All values are area %

The data showed that in ethanol the only radio impurity formed is the ¹⁸F. The ¹⁸F is PGP-5912 being displaced over time, the levels rising initially and then plateau over time. Ethanol is a radical scavenger but in this study fluorine is predominantly displaced within four hours post EOS and is demonstrated to occur in the presence of the ethanol radical scavenger. The displacement of a halogen from a pyridine ring is known to occur under harsh basic conditions such as with sodium methoxide or ethoxide solutions. These can only be present in an aqueous medium. In the case of the elution of the HLB cartridge with during processing, any water remaining in the HLB can be incorporated into the elution band.

Examination of the chromatograms confirms that it is difficult to integrate the broad ¹⁸F peak and that the plateau in the formation of ¹⁸F could be caused by the challenge associated with its integration. When TEAF (tetraethyl ammonium fluoride) was added to the product at the 10 hour time point, the peak was sharper and easier to integrate. The levels of the radiolysis impurity at this point were at 7.35% versus 4.55% when TEAF was not added to the sample. It is worth noting that the ethanol eluting the HLB contains 2-3 Ci of ¹⁸F-DCFPyL as it exits the HLB cartridge suggesting the potential for radiolysis in ethanol alone is much higher than in the bulk product solution. Chromatograms of the injection product at the 10 hour time point, with and without the TEAF, are given in FIG. 5.

Subsequent pilot studies were performed where the maximum ¹⁸F handling, to minimize radiation exposure, is limited to approximately 2 Ci. In order to achieve radioactive concentrations of approximately 120 mCi/mL, the labeled product was eluted from the HLB cartridge used for solvent exchange and delivered using a Nitrogen push to the receiving vial which was preloaded with a minimum of 1.3 mL of formulation matrix (0.9% Sodium Chloride, USP, or Ascorbic Acid, USP). The product was then assayed using a Capintec dose calibrator and diluted to achieve the desired radioactive concentration. This results in a product having a higher ethanol level (approximately 20%). While ethanol is known to provide some protection against radiolysis, studies at high ethanol levels nevertheless provide an approach to the initial assessment of the stabilization using ascorbic acid.

A control lot was produced without incorporation of ascorbic acid and had a final radiochemical concentration of 147 mCi/mL and a 29.8% ethanol level. The product was sampled every 30 minutes and assayed with an extended gradient added to allow detection of hydrophobic impurities. The product was delivered from the HLB into a vial containing 1.3 mL of 0.9% Sodium Chloride, USP. The product was assayed and diluted to achieve the desired radioactive concentration with 0.9% Sodium Chloride, USP. The initial radiochemical purity (RCP) was 94% and the T=4 hour RCP was 89%, well below the ≥95% RCP product specification. In addition to free ¹⁸F, there were two additional radioactive impurities that elute before the ¹⁸F-DCFPyL peak at initial time point chromatogram but do not appear to grow over time. These may be process impurities as opposed to ongoing radiolytic degradation. There are two additional peaks below the reportable limit of 0.3% w/w, that elute after the ¹⁸F-DCFPyL product peak. Data are provided in Table 37. The T=0 hour chromatogram is provided in FIG. 6.

A lot was produced with 35 mL of 100 mg/mL pH 2.0 ascorbic acid solution preloaded into the collection vial. The HLB cartridge was also rinsed with 5 mL of the 100 mg/mL pH 2.0 ascorbic acid solution. Then 25 mL of 100 mg/mL pH 2.0 ascorbic acid was filled into the original delivery solution vial (Position F, FIG. 13). Once the product was captured on the HLB this ascorbic acid solution was used to wash the HLB and do the solvent exchange instead of SWFI. Then the ¹⁸F-DCFPyL was delivered from the HLB into the empty product vial in 100% ethanol. The product was assayed. In order to analyze the sample, 12. 5 μL of the product in 100% ethanol needed was diluted with 237.5 μL of saline. Samples were taken every 30 minutes and assayed. Tetraethylammonium fluoride, 44 mg/mL, was added to the samples for the radiochemical purity HPLC analysis in order to reduce column retention and sharpen the ¹⁸F peak to achieve better quantitation. The product had a radiochemical concentration of 256 mCi/mL. The initial RCP was 99.7% and the ¹⁸F impurity was 0.29%. Only one of the two impurities, seen in the control lot, eluting before the ¹⁸F-DCFPyL peak was sporadically present ranging from <LLQ to 0.35%, but were not seen consistently at reportable levels (>0.3%). The RCP was 98.0% at T=4 hours. Data for this lot are provided in Table 38. This clearly shows that radiolysis was greatly reduced when ascorbic acid was added to the collection vial. The T=0 and T=4 hour chromatograms are shown overlaid in FIG. 8.

A lot was produced with 35 mL of 100 mg/mL pH 2.0 ascorbic acid solution preloaded into the collection vial. The HLB cartridge was also rinsed with 5 mL of the 100 mg/mL pH 2.0 ascorbic acid solution. Finally, the ¹⁸F-DCFPyL was collected in a final product vial preloaded with 50 mg/mL of pH 5.8 ascorbic acid solution, assayed by Capintec and diluted to the desired concentration with 50 mg/mL of pH 5.8 ascorbic acid solution. The product was sampled every 30 minutes and assayed and had tetraethylammonium fluoride, 44 mg/mL, added. The product had a radiochemical concentration of 110 mCi/mL and a 24% (w/w) ethanol level. The initial RCP was 99.7% and the ¹⁸F impurity was <LLQ. The two impurities eluting before the ¹⁸F-DCFPyL peak, seen in the control lot, were present at levels ranging from <LLQ to 0.37%, but were not consistently at reportable levels (>0.3%). The RCP remained above 99% at T=4 hours. Data for this lot are provided in Table 39. The T=0 and T4 hour chromatograms are shown overlaid in FIG. 9. This clearly shows that radiolysis was greatly reduced with the use of ascorbic acid in the collection vessel, wash solution and in the FPV. However, the contribution of ethanol to the stabilization of ¹⁸F-DCFPyL was unknown.

TABLE 314
Radiolysis of 147 mCi of ¹⁸F-DCFPyL/mL in 0.9% Sodium Chloride, USP

| | T = 0^{a} | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL^{b} | 94.17 | 90.65 | 90.71 | 89.59 | 89.50 | 88.79 | 88.94 | 88.90 | 89.06 |
| Free ¹⁸F^{b} | 4.68 | 7.73 | 7.69 | 8.78 | 8.67 | 9.66 | 9.49 | 9.56 | 9.27 |
| RT 5.0 min^{b} | 0.50 | 0.65 | 0.48 | 0.61 | 0.77 | 0.62 | 0.70 | 0.59 | 0.71 |
| RT 5.9 min^{b} | 0.64 | 0.97 | 1.11 | 1.03 | 1.05 | 0.93 | 0.87 | 0.95 | 0.96 |

^{a}All times are in hours
^{b}All values are area %

TABLE 315
Radiolysis of 256 mCi of ¹⁸F-DCFPyL/mL in Ethanol

| 256 mCi/mL^{a} | T = 0^{b} | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL^{c} | 99.71 | 99.60 | 98.68 | 98.58 | 98.20 | 98.71 | 98.46 | 97.96 | 97.95 |
| Free ¹⁸F^{c} | 0.29 | 0.40 | 0.31 | 0.31 | 0.31 | 0.31 | 0.29 | 0.42 | 0.40 |
| RT 5.0 min^{c} | <QL | <QL | 0.35 | 0.28 | 0.50 | <QL | <QL | <QL | <QL |
| RT 12.0 min^{c} | <QL | <QL | 0.35 | 0.46 | 0.52 | 0.52 | 0.69 | 0.87 | 0.96 |
| RT 12.2 min^{c} | <QL | <QL | 0.31 | 0.37 | 0.47 | 0.46 | 0.57 | 0.75 | 0.69 |

^{a}The collection vial was preloaded with 35 mL of 100 mg/mL pH 2.0 ascorbic acid and receives approximately 5 mL in the peak cut. The HLB cartridge was washed with 5 mL of 100 mg/mL pH 2.0 ascorbic acid to leave it protected with the radical scavenger solution, and the product was delivered in ethanol from the HLB into a vial. The product was assayed.
^{b}All times are in hours
^{c}All values are area %

TABLE 316
Radiolysis of 110 mCi of ¹⁸F-DCFPyL/mL in 50 mg/mL pH 5.8 ascorbic acid

| 110 mCi/mL^{a} | T = 0^{b} | T = 0.5 | T = 1.0 | T = 1.5 | T = 2.0 | T = 2.5 | T = 3.0 | T = 3.5 | T = 4.0 |
|---|---|---|---|---|---|---|---|---|---|
| ¹⁸F-DCFPyL^{c} | 99.73 | 99.72 | 96.74 | 99.66 | 99.76 | 99.66 | 99.30 | 99.46 | 99.43 |
| Free ¹⁸F^{c} | <QL | <QL | <QL | <QL | <QL | <QL | 0.35 | 0.24 | 0.26 |
| RT 5.0 min^{c} | 0.27 | 0.28 | 0.26 | 0.34 | 0.24 | 0.34 | 0.35 | 0.31 | 0.31 |
| RT 5.9 min^{c} | <QL | <QL | <QL | <QL | <QL | <QL | <QL | <QL | <QL |

^{a}The collection vial was preloaded with 35 mL of 100 mg/mL pH 2.0 ascorbic acid and receives approximately 5 mL in the peak cut. The HLB cartridge was washed with 5 mL of 100 mg/mL pH 2.0 ascorbic acid to leave it protected with the radical scavenger solution, and the product was delivered from the HLB into a vial containing 1.3 mL of 50 mg/mL pH 5.8 ascorbic acid. The product was assayed and diluted to the desired radioactive concentration with 50 mg/mL pH 5.8 ascorbic acid.
^{b}All times are in hours
^{c}All values are area %

### Initial Lots Examining Addition of Ascorbic Acid to the PYLARIFY Formulation

The stability of ¹⁸F-DCFPyL Injection was studied at activities greater than the currently approved product limit of 80 mCi/mL. Ascorbic acid was added as a radio-protectant to the FPV to demonstrate the feasibility of stabilizing product and then also to the delivery solution vial to determine if additional stabilization was achievable by adding ascorbate to the saline (Position F, FIG. 13) used to deliver the product from the HPB cartridge to the FPV as indicated in Table 40. The last lot in this sequence examined the stability of the peak cut in the collection vial (not delivered to FPV containing ascorbate). A listing of solution compositions for these studies is provided in Table 41. Starting activity for the labelling reaction was targeted at 15 Ci with the goal of producing product at radioactive concentrations>120 mCi/mL.

Three lots were produced incorporating pH 6.0 ascorbic acid into the final product vial (FPV). The lots were prepared using ¹⁸F starting activities of 17.6 Ci, 13.5 Ci and 13.4 Ci which resulted in final activity concentrations of 166 mCi/mL, 120 mCi/mL and 127 mCi/mL, respectively. Samples were assayed using the radiochemical HPLC assay at 0, 2, 4, 6 and 10 hours post end of synthesis (EOS). The T=0, T=4 and T=10 hour results for Lot 220816PyL, Lot 220817PyL, and Lot 220818PyL are provided in Table 42. Plots of the results for RCP, ¹⁸F, peaks at retention times (RT) of 6.0, 9.8 and 10.6 minutes are provided in FIGS. 16-20, respectively, including additional timepoint data not listed in Table 42.

The data and plot for total RCP as a function of time indicates ¹⁸F-DCFPyL is more stable at lower radioactive concentration (T=10 hour RCP of 95.5% and 96.9% at 166 and 120 mCi/mL, respectively) when the ascorbic acid level is at 5.6 mg/mL, while decreasing the ascorbic acid concentration to 2.8 mg/mL for formulations at approximately the same radiochemical concentration (120 to 127 mCi/mL) results in lower RCP (96.9% versus 96.1% RCP). The lot formulated at 127 mCi/mL in 2.8 mg/mL pH 6.0 ascorbic acid, had slightly better stability (approximately 0.5% RCP difference) when compared to the lot formulated in 5.6 mg/mL pH 6.0 ascorbic acid (165 mCi/mL), consistent with greater radiolysis at higher radioactive concentration. The same lot had slightly poorer stability (approximately 0.5% difference) when compared to the lot formulated in 5.6 mg/mL pH 6.0 ascorbic acid but having similar radioactive concentration (120 mCi/mL). This is consistent with the lower concentration of ascorbic acid in the formulation providing less radioprotection and is also evident from the plot of ¹⁸F formation for the lots.

The plots for the peaks at retention times of 6.0, 9.8 and 10.6 minutes range between 0.4 and 0.6% at T=0 minutes and show small changes (<0.2%) from their initial levels over the 10 hour course of the study. A representative chromatogram is provided in FIG. 20. The lack of significant change from T=0 to T=10 hours may indicate these three peaks are process impurities or formed before transfer to the FPV. Overall, the three lots did not achieve the desired RCP (>99%) at T=0 hours, with the lower RCP results being driven potentially by the presence of process impurities. However, the lots do demonstrate addition of ascorbic acid at both 2.8 and 5.6 mg/mL to the FPV stabilizes ¹⁸F-DCFPyL degradation even at concentrations as high as 165 mCi/mL.

Addition of ascorbic acid (5 mg/mL) to the saline vial (position F, FIG. 13) that is used to deliver the product from the HLB cartridge to the final product vial (FPV) in addition to adding ascorbic acid (5 mg/mL) to the 20 mL of saline prefilled into the FPV was examined with high 18F-DCFPyL concentrations. Lot 220819PyL was produced with a starting activity of 13.7 Ci, and the radioactive concentration of the product was 134 mCi/mL at EOS. The RCP and ¹⁸F results for Lot 220819PyL (5 mg/mL ascorbic acid in both FPV and delivery solution) and Lot 220817PyL (10 mg/mL ascorbic acid in just the FPV diluted to 5 mg/mL with addition of delivery solution) are similar 97.9% versus 97.8% and 0.52% versus 0.6%, respectively, and indicate delivery of the product from the HLB using pH 6.0 ascorbic acid (5 mg/mL) does not offer additional stabilization, Table 42.

Lot 220908PyL (HCI) was produced to determine if the impurities at approximate RT's of 6.0, 9.8 and 10.6 were present in the peak cut from the semi-prep HPLC purification. The peak cut was delivered into the collection vial containing 35 mL SWFI. The contents of the collection vial were immediately delivered to the FPV containing 16 mL of 0.9% saline and the contents analyzed immediately without further processing. This provided for assessment of whether the impurities observed are process or degradation impurities. The peaks at RT's of 6.0, 9.8, and 10.6 minutes were present at 2.13, 1.42, and 0.72%, respectively and are consistent with levels observed in lots produced with ascorbic acid in the FPV, Table 42. The radiochemical chromatogram is provided in FIG. 22.

TABLE 17
Position of ascorbic acid additions for initial FPV ascorbic acid studies

| Vial | | Lot 220816PyL | Lot 220817PyL | Lot 220818PyL | Lot 220819PyL | Lot 220908PyL |
|---|---|---|---|---|---|---|
| Collection | Ascorbic | − | − | − | − | − |
| vial | Acid | | | | | |
| (Position I, | Saline | − | − | − | − | − |
| FIG. 13) | SWFI | + | + | + | + | + |
| Delivery | Ascorbic | − | − | − | + | not applicable^{a} |
| solution vial | Acid | | | | | |
| (Position F, | Saline | + | + | + | + | not applicable^{a} |
| FIG. 13) | SWFI | − | − | − | − | not applicable^{a} |
| FPV | Ascorbic Acid | + | + | + | + | not applicable^{a} |
| | Saline | + | + | + | + | not applicable^{a} |
| | SWFI | − | − | − | − | not applicable^{a} |

^{a}This lot was designed to examine radiochemical impurities in the semi-prep HPLC peak cut and did not perform the solvent exchange or load to the FPV.

TABLE 18
Ascorbic acid, pH 6 added to the FPV

| Vial | Lot 220816PyL | Lot 220817PyL | Lot 220818PyL | Lot 220819PyL | Lot 220908PyL |
|---|---|---|---|---|---|
| Collection vial | 35 mL of SWFI | 35 mL of SWFI | 35 mL of SWFI | 35 mL of SWFI | 35 mL of SWFI |
| (Position I, | | | | | |
| FIG. 13) | | | | | |
| Delivery | Fill: 20 mL of | Fill: 20 mL of | Fill: 20 mL of | Fill: 20 mL of | not applicable^{a} |
| solution vial | 0.9% saline | 0.9% saline | 0.9% saline | 5 mg/mL | |
| (Position F, | Delivery: 16 | Delivery: 16 | Delivery: 16 | ascorbic acid in | |
| FIG. 13) | mL to FPV | mL to FPV | mL to FPV | 0.9% saline | |
| | | | | Delivery: 16 | |
| | | | | mL to FPV | |
| FPV | 20 mL of 10 | 20 mL of 10 | 20 mL of 5 | 20 mL of 5 | 20 mL of 0.9% |
| | mg/mL ascorbic | mg/mL ascorbic | mg/mL ascorbic | mg/mL ascorbic | saline |
| | acid, pH 6 in | acid, pH 6 in | acid, pH 6 in | acid, pH 6 in | |
| | 0.9% saline | 0.9% saline | 0.9% saline | 0.9% saline | |
| Ascorbic acid | 5.6 mg/mL^{b} | 5.6 mg/mL^{a} | 2.8 mg/mL^{c} | 5.6 mg/m^{a}L | not applicable |
| concentration | | | | | |
| after delivery | | | | | |
| of all | | | | | |
| components | | | | | |

^{a}This lot was designed to examine radiochemical impurities in the semi-prep HPLC peak cut and did not perform the solvent exchange or load to the FPV.
^{a}16 mL saline plus 20 mL of 10 mg/mL ascorbic acid = (20 mL × 10 mg/mL)/36 mL = 5.6 mg/mL ascorbic acid
^{b}16 mL saline plus 20 mL of 5 mg/mL ascorbic acid = (20 mL × 5 mg/mL)/36 mL = 2.8 mg/mL ascorbic acid

TABLE 19
Results for initial lots examining the addition of ascorbic acid to the PYLARIFY formulation

| | Lot 220816PyL | Lot 220817PyL | Lot 220818PyL | Lot 220819PyL | Lot 220908PyL |
|---|---|---|---|---|---|
| Starting Activity | 17.6 Ci | 13.4 Ci | 13.4 Ci | 13.7 Ci | 13.6 Ci |
| mCi/mL | 166 | 120 | 127 | 134 | 115 |
| Collection Vial | SWFI | SWFI | SWFI | SWFI | n/a^{a} |
| Delivery Solution Vial | 0.9% Sodium | 0.9% Sodium | 0.9% Sodium | 5 mg/mL pH 6.0 in | n/a^{a} |
| | Chloride, USP | Chloride, USP | Chloride, USP | 0.9% Sodium | |
| | | | | Chloride, USP | |
| FPV | 10 mg/mL, pH 6.0 in | 10 mg/mL, pH 6.0 in | 5 mg/mL, pH 6.0 in | 5 mg/mL, pH 6.0 in | n/a^{a} |
| | 0.9% Sodium | 0.9% Sodium | 0.9% Sodium | 0.9% Sodium | |
| | Chloride, USP | Chloride, USP | Chloride, USP | Chloride, USP | |
| Product | 5.6 mg/mL, pH 6.0 | 5.6 mg/mL, pH 6.0 | 2.8 mg/mL, pH 6.0 | 5 mg/mL, pH 6.0 | n/a |
| ascorbic acid | | | | | |
| concentration | | | | | |

| | T = 0^{b} | T = 4 | T = 10 | T = 0 | T = 4 | T = 10 | T = 0 | T = 4 | T = 10 | T = 0 | T = 4 | T = 10 | T = 0 | T = 4 | T = 10 |
|---|---|---|---|---|---|---|---|---|---|---|---|---|---|---|---|
| RCP^{c} | 97.6 | 96.0 | 95.5 | 97.8 | 96.9 | 96.9 | 97.8 | 96.2 | 96.1 | 97.9 | 97.0 | nt^{d} | 97.6 | 81.4 | nt^{d} |
| ¹⁸F^{c} | 0.95 | 2.42 | 3.05 | 0.62 | 1.44 | 1.86 | 0.53 | 2.19 | 2.78 | 0.52 | 1.59 | nt^{d} | 1.12 | 11.07 | nt^{d} |
| RT 6.0 min^{c} | 0.39 | 0.40 | nd^{e} | 0.52 | 0.43 | nd^{e} | 0.44 | 0.46 | nd^{e} | 0.46 | 0.38 | nt^{d} | 0.64 | 2.13 | nt^{d} |
| RT 9.8 min^{c} | 0.52 | 0.49 | 0.68 | 0.50 | 0.54 | 0.52 | 0.55 | 0.53 | 0.45 | 0.51 | 0.48 | nt^{d} | nd^{e} | 1.42 | nt^{d} |
| RT 10.6 min^{c} | 0.57 | 0.68 | 0.76 | 0.52 | 0.68 | 0.68 | 0.67 | 0.62 | 0.67 | 0.62 | 0.52 | nt^{d} | nd^{e} | 0.72 | nt^{d} |

^{a}n/a = not applicable
^{b}All times are in hours
^{c}All values are area %
^{d}nt = not tested
^{e}nd = not detected
^{d}In saline degradation is greater at an activity >80 mCi/mL. So this sample had greater degradation. To keep data set consistent peak at RT 6.4 at T = 0 hr was not tabulated. The level was 0.61%. At T = 4 hrs there were additional peaks at RT 5.0 (0.29%), RT 6.4 (1.99%), RT 8.0 (0.45%), RT 9.2 (0.55%).

### Addition of Ascorbic Acid to the Collection Vial (Position I, FIG. 13)

Further studies were designed to determine if the initial RCP for PYLARIFY could be increased through the stabilization of the purified product prior to the solvent exchange step on the HLB cartridge. Based on the study completed previously, this seemed like a way to stabilize the product before it gets loaded on the HLB. Ascorbic acid was added to the collection vial (Position I, FIG. 13) and/or to the delivery solution vial in addition to the FPV (Position F, FIG. 13) as indicated in Table 43. A last lot in this sequence examined adding the ascorbate to the formulation only via the delivery solution vial, thus eliminating the preload of ascorbic acid to the FPV. A listing of solution compositions for these studies is provide in Table 44.

Lot 220909Pyl (HCI) was produced using pH 6.0 ascorbic acid loaded into both the collection vial and the FPV. The concentration of ascorbic acid in the FPV after delivery of the product from the HLB cartridge was 5.6 mg/mL. The pH of the product in the collection vial after delivery of the peak cut was 4.7. The radiochemical concentration in the FPV was 89 mCi/mL, which was significantly lower than the >120 mCi/mL expected. The initial RCP was 99%, and >97% at 4 hours, Table 45. While this study demonstrated good stability of the ¹⁸F-DCFPyL, the radioactive concentration was not high enough to support an assessment of stability at high radioactive concentrations. The impurities were below reportable levels (<0.3%) at T=4 hours, FIG. 24. The results from this study indicate the impurities can be decreased by stabilizing the product with ascorbate prior to solvent exchange. During lot 220909Pyl (HCl) production, high levels of radioactivity were detected by the AIO waste line radiodetector during the solvent exchange indicating the product was not well retained on the HLB cartridge. It is hypothesized that ¹⁸F-DCFPyL retention on the HLB cartridge decreases as the pH increases, consistent with increased ionization of ¹⁸F-DCFPyL as the pH is increased.

Lot 220914PyL (HCI) was produced with 5.0 mg/mL pH 6 ascorbic acid in the delivery solution vial and in the FPV. The collection vial contained 5.0 mg/mL pH 2 ascorbic acid, to test whether the pH of the sample loaded onto the HLB cartridge impacts ¹⁸F-DCFPyL retention. The radioactive concentration of the product was 159 mCi/mL, the RCP was 99.0% and 97.6% at T=0 and T=4 hours, respectively. The radiochemical impurities were not at reportable levels, Table 45. The T=4 hour radiochromatogram is provided in FIG. 25. The RCP results are similar to the results for product produced without adding the ascorbic acid to the delivery solution vial (Lot 220909PyL; 99.6% and 98.8% at T=0 and T=4 hours, respectively, Table 45). Lowering the collection vial pH to 2 resulted in retention of the product with virtually no ¹⁸F-DCFPyL being observed in the HLB cartridge washes (based on low radioactivity being detected by the AIO waste line radiodetector).

Lot 220927PyL (HCI) was prepared using 10.0 mg/mL pH 6.0 ascorbic acid in the FPV vial and 5.0 mg/mL pH 2 ascorbic acid loaded into the collection vial. The radiochemical concentration of the lot was 127 mCi and the RCP was 99.0% and 98.1% at T=0 and T=10 hours, respectively, Table 45. The radiochemical impurities were not at reportable levels though they were near the limit (0.3%); the radiochromatogram is provided in FIG. 26. The results for this lot are similar to the results for Lot 220914PyL (99.0 and 98.6 at T=0 and T=4 hours, respectively [no ascorbic acid in the delivery vial], Table 45), indicating the presence of ascorbic acid in the delivery vial does not improve stability relative to only adding it to the collection vial.

Lot 220928PyL (HCI) was prepared using 10 mg/mL ascorbic acid, pH 6.0 in the delivery solvent vial, 5 mg/mL pH 2.0 ascorbic acid in SWFI in the collection vial and saline in the FPV. The radiochemical concentration was 138 mCi/mL, the RCP was 99.7%, 98.3% and 97.8% at T=0, T=4 and T=10 hours, reproducing the results of Lot 220927Pyl, 99.0%, 98.0% and 98.0%, at T=0, T=4 and T=10 hours, Table 45. There were no reportable radiochemical impurities present; the radiochemical chromatogram is provided in FIG. 27. Addition of ascorbic acid to the delivery vial offers no advantage over putting the ascorbic acid in the FPV (Final Product Vial) vial.

TABLE 20
Position of ascorbic acid additions for initial Collection Vial ascorbic acid studies

| | | Lot | Lot | Lot | Lot |
|---|---|---|---|---|---|
| Vial | | 220909Pyl | 220914Pyl | 220927PyL | 220928PyL |
| Collection vial | Ascorbic Acid | + | + | + | + |
| (Position I, | Saline | − | − | − | − |
| FIG. 13) | SWFI | + | + | + | + |
| Delivery solution | Ascorbic Acid | − | + | − | + |
| vial (Position F, | Saline | + | + | + | + |
| FIG. 13) | SWFI | − | − | − | − |
| FPV | Ascorbic Acid | + | + | + | − |
| | Saline | + | + | + | + |
| | SWFI | − | − | − | − |

TABLE 21
Studies with ascorbic acid added to collection vial (Position I, FIG. 13)

| Vial | Lot 220909Pyl | Lot 220914Pyl | Lot 220927PyL | Lot 220928PyL |
|---|---|---|---|---|
| Collection vial | 35 mL of 5.0 | 35 mL of 5.0 | 35 mL of 5.0 | 35 mL of 5.0 |
| (Position I, | mg/mL pH 6.0 | mg/mL pH 2.0 | mg/mL pH 2.0 | mg/mL pH 2.0 |
| FIG. 13) | ascorbic acid | ascorbic acid | ascorbic acid | ascorbic acid |
| | in SWFI | in SWFI | in SWFI | in SWFI |
| Delivery | Fill: 20 mL of 0.9% | Fill: 20 mL of 5.0 | Fill: 20 mL of 0.9% | Fill: 20 mL of 10.0 |
| solution vial | Sodium Chloride, | mg/mL pH 6.0 | Sodium Chloride, | mg/mL pH 6.0 |
| (Position F, | USP | ascorbic acid in | USP | ascorbic acid in 0.9% |
| FIG. 13) | Delivery: 16 mL | 0.9% Sodium | Delivery: 16 mL | Sodium Chloride, USP |
| | to FPV | Chloride, USP | to FPV | Delivery: 16 mL |
| | | Delivery: 16 mL | | to FPV |
| | | to FPV | | |
| FPV | 20 mL of 10.0 | 20 mL of 5.0 | 20 mL of 10.0 | 20 mL 0.9% Sodium |
| | mg/mL ascorbic acid, | mg/mL ascorbic | mg/mL ascorbic acid, | Chloride, USP |
| | pH 6.0 in 0.9% | acid, pH 6.0 in 0.9% | pH 6.0 in 0.9% | |
| | Sodium Chloride, | Sodium Chloride, | Sodium Chloride, | |
| | USP | USP | USP | |
| Ascorbic acid | 5.6 mg/mL^{a} | 5.0 mg/mL^{b} | 5.6 mg/mL^{a} | 4.4 mg/mL^{c} |
| concentration | | | | |
| after delivery | | | | |
| of all | | | | |
| components | | | | |

^{a}16 mL 0.9% Sodium Chloride, USP, plus 20 mL of 10 mg/mL ascorbic acid = (20 mL × 10 mg/mL)/36 mL = 5.6 mg/mL ascorbic acid
^{b}16 mL of 5.0 mg/mL ascorbic acid in 0.9% Sodium Chloride, USP, plus 20 mL of 5 mg/mL ascorbic acid in 0.9% Sodium Chloride, USP = [(16 mL × 5 mg/mL) + (20 mL × 5 mg/mL)/36 mL = 5.0 mg/mL ascorbic acid
^{c}16 mL of 10.0 mg/mL ascorbic acid in 0.9% Sodium Chloride, USP, plus 20 mL of 0.9% Sodium Chloride, USP = [(16 mL × 5 mg/mL) + (20 mL × 5 mg/mL)/36 mL = 4.4 mg/mL ascorbic acid

TABLE 22
Addition of ascorbic acid to the collection vial

| | Lot 220909Pyl | Lot 220914Pyl | Lot 220927PyL | Lot 220928PyL |
|---|---|---|---|---|
| Starting activity | 13.7 Ci | 14.0 | 13.7 | 13.5 |
| mCi/mL | 89 | 160 | 127 | 138 |
| Collection Vial | 5.0 mg/mL pH 6.0 | 5.0 mg/mL pH 2.0 | 5.0 mg/mL pH 2.0 | 5.0 mg/mL pH 2.0 |
| | ascorbic acid in SWFI | ascorbic acid in SWFI | ascorbic acid in SWFI | ascorbic acid in SWFI |
| Delivery Solution | 0.9% Sodium Chloride, | 5.0 mg/mL pH 6.0 | 0.9% Sodium Chloride, | 10.0 mg/mL pH 6.0 |
| Vial | USP | ascorbic acid in 0.9% | USP | ascorbic acid in 0.9% |
| | | Sodium Chloride, USP | | Sodium Chloride, USP |
| FPV | 10.0 mg/mL ascorbic | 5.0 mg/mL ascorbic | 10.0 mg/mL ascorbic | 0.9% Sodium Chloride, |
| | acid, pH 6.0 in 0.9% | acid, pH 6.0 in 0.9% | acid, pH 6.0 in 0.9% | USP |
| | Sodium Chloride, USP | Sodium Chloride, USP | Sodium Chloride, USP | |
| Product | 5.6 mg/mL | 5.0 mg/mL | 5.6 mg/mL | 4.4 mg/mL |
| ascorbic acid | | | | |
| concentration | | | | |

| | T = 0^{a} | T = 4 | T = 10 | T = 0 | T = 4 | T = 10 | T = 0 | T = 4 | T = 10 | T = 0 | T = 4 | T = 10 |
|---|---|---|---|---|---|---|---|---|---|---|---|---|
| RCP^{b} | 99.6 | 98.8 | nt^{c} | 99.0 | 97.6 | nt | 99.1 | 98.0 | 98.1 | 99.7 | 98.3 | 97.9 |
| 18F^{b} | 0.39 | 1.17 | nt | 0.97 | 2.42 | nt | 0.59 | 1.70 | 1.95 | 0.32 | 1.70 | 2.09 |
| RT 6.0 min^{b} | nd^{d} | nd^{d} | nt | nd^{d} | nd^{d} | nt | 0.31 | 0.35 | nd^{d} | nd^{d} | nd^{d} | nd^{d} |
| RT 9.8 min^{b} | nd^{d} | nd^{d} | nt | nd^{d} | nd^{d} | nt | nd^{d} | nd^{d} | nd^{d} | nd^{d} | nd^{d} | nd^{d} |
| RT 10.6 min^{b} | nd^{d} | nd^{d} | nt | nd^{d} | nd^{d} | nt | nd^{d} | nd^{d} | nd^{d} | nd^{d} | nd^{d} | nd^{d} |

^{a}All times are in hours
^{b}All values are area %
nt = not tested
^{d}nd = not detected

### Effect of FPV pH and Ascorbic Acid Concentration on Stability for the Target Formulation

Based on the above studies that showed a pH drift from an initial target pH of 6.0 at EOS to approximately pH 6.8 at T=10 hours, a pH target for the formulation was lowered to 5.5 for the ascorbic acid formulation. Production can include loading the collection vial with 35 mL of 10 mg/mL, pH 2 ascorbic acid in 0.9% Sodium Chloride, USP, and can preload 20 mL of 10 mg/mL pH 5.5 ascorbic acid in 0.9% Sodium Chloride, USP, into the FPV. The ascorbic acid concentration in the product can be 5.6 mg/mL after delivery of the purified product from the HLB solvent exchange cartridge to the FPV.

The impact of FPV pH on the stability of ¹⁸F-DCFPyL was studied at the target pH and the pH specification limits of 4.5 and 7.0. Lots were prepared at pH 4.5, 5.5 and 7.0 and samples were analyzed at 0, 2, 4, 6, and 10 hours for RCP and 0 and 10 hours for pH. See Table 46 for a summary of the composition of the delivery solution vial, the collection vial and the FPV.

Lot 221025Pyl was prepared with 10 mg/mL pH 2.0 ascorbic acid in the collection vial and 10.0 mg/mL pH 5.5 ascorbic acid in the FPV. The radiochemical concentration of the product was 133 mCi/mL. The RCP was 98.9% at 0 hours and 97.4% at 10 hours, Table 47. There was one radiochemical impurity present at ˜0.5% through 4 hours that was not present after 4 hours. No other impurities were present at a reportable level; the T=10 hour chromatogram is provided in FIG. 28.

Lot 221026Pyl was prepared with 10 mg/mL pH 2.0 ascorbic acid in the collection vial and 10.0 mg/mL pH 4.5 ascorbic acid in the FPV. The radiochemical concentration of the product was 133 mCi/mL. The RCP was 99.4% at 0 hours and 98.0% at 10 hours, Table 47. There were no reportable radiochemical impurities present throughout the study. The T=10 hour chromatogram is provided in FIG. 29.

Lot 230124Pyl was prepared with 10 mg/mL pH 2.0 ascorbic acid in the collection vial and 10.0 mg/mL pH 7.5 ascorbic acid in the FPV. The radiochemical concentration of the product was 162 mCi/mL. The RCP was 99.2% at 0 hours and 97.4% at 10 hours, Table 47. There were no reportable radiochemical impurities present throughout the study. The T=10 hour chromatogram is provided in FIG. 30.

TABLE 23
Studies of PYLARIFY product at different pH's

| Vial | Lot 221026Pyl | Lot 221025Pyl | Lot 230124PyL |
|---|---|---|---|
| Collection vial | 35 mL of 10.0 mg/mL pH 2.0 | 35 mL of 10.0 mg/mL pH 2.0 | 35 mL of 10.0 mg/mL pH 2.0 |
| (Position I, | ascorbic acid in 0.9% | ascorbic acid in 0.9% | ascorbic acid in 0.9% |
| FIG. 13) | Sodium Chloride, USP | Sodium Chloride, USP | Sodium Chloride, USP |
| Delivery | Fill: 20 mL of 0.9% | Fill: 20 mL of 0.9% | Fill: 20 mL of 0.9% |
| solution vial | Sodium Chloride, USP | Sodium Chloride, USP | Sodium Chloride, USP |
| (Position F, | Delivery: 16 mL to FPV | Delivery: 16 mL to FPV | Delivery: 16 mL to FPV |
| FIG. 13) | | | |
| FPV | 20 mL of 10.0 mg/mL | 20 mL of 10.0 mg/mL | 20 mL of 10.0 mg/mL |
| | ascorbic acid, | ascorbic acid, | ascorbic acid, |
| | pH 4.5 in 0.9% | pH 5.5 in 0.9% | pH 7.0 in 0.9% |
| | Sodium Chloride, USP | Sodium Chloride, USP | Sodium Chloride, USP |
| Product | 5.6 mg/mL, pH 4.5 | 5.6 mg/mL, pH 5.5 | 5.6 mg/mL, pH 7.0 |
| ascorbic acid | | | |
| concentration^{a} | | | |
| and pH | | | |

^{a}16 mL 0.9% Sodium Chloride, USP, plus 20 mL of 10 mg/mL ascorbic acid = (20 mL × 10 mg/mL)/36 mL = 5.6 mg/mL ascorbic acid

TABLE 24
Data for PYLARIFY produced at different pH's

| | Lot 221026Pyl | | Lot 221025Pyl | | Lot 230124PyL | |
|---|---|---|---|---|---|---|
| Timepoint (hr) | T = 0 | T = 10 | T = 0 | T = 10 | T = 0 | T = 10 |
| pH | 4.9 | 5.0 | 5.9 | 6.3 | 7.6 | 6.8 |

| Starting activity | 14.7 | 14.7 | 16.5 |
|---|---|---|---|
| mCi/mL | 133 | 133 | 162 |
| Collection Vial | 10.0 mg/mL pH 2.0 ascorbic | 10.0 mg/mL pH 2.0 ascorbic | 10.0 mg/mL pH 2.0 ascorbic |
| | acid in 0.9% Sodium | acid in 0.9% Sodium | acid in 0.9% Sodium |
| | Chloride, USP | Chloride, USP | Chloride, USP |
| Delivery | 0.9% Sodium Chloride, USP | 0.9% Sodium Chloride, USP | 0.9% Sodium Chloride, USP |
| Solution Vial | | | |
| FPV | 10.0 mg/mL ascorbic acid, pH 4.5 | 10.0 mg/mL ascorbic acid, pH 5.5 | 10.0 mg/mL ascorbic acid, pH 7.0 |
| | in 0.9% Sodium Chloride, USP | in 0.9% Sodium Chloride, USP | in 0.9% Sodium Chloride, USP |
| Product | 5.6 mg/mL | 5.6 mg/mL | 5.6 mg/mL |
| ascorbic acid | | | |
| concentration | | | |

| Timepoint (hr) | T = 0^{a} | T = 2 | T = 4 | T = 6 | T = 10 | T = 0 | T = 2 | T = 4 | T = 6 | T = 10 | T = 0 | T = 2 | T = 4 | T = 6 | T = 10 |
|---|---|---|---|---|---|---|---|---|---|---|---|---|---|---|---|
| RCP^{b} | 99.4 | 98.8 | 98.3 | 98.1 | 98.0 | 98.9 | 98.2 | 98.1 | 97.9 | 97.4 | 99.2 | 98.2 | 97.8 | 97.3 | 97.4 |
| 18F^{b} | 0.61 | 1.22 | 1.68 | 1.92 | 2.04 | 0.71 | 1.33 | 1.89 | 2.10 | 2.08 | 0.83 | 1.84 | 2.24 | 2.71 | 2.65 |
| RT 6.0 mi^{b} | nd^{c} | nd^{c} | nd^{c} | nd^{c} | nd^{c} | 0.42 | 0.52 | nd^{c} | nd^{c} | 0.53 | nd^{c} | nd^{c} | nd^{c} | nd^{c} | nd^{c} |
| RT 9.8 min^{b} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} |
| RT 10.6 min^{b} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} |

^{a}All times are in hours,
^{b}All values are area %,
^{c}nd = not detected

### Discussion of pH Studies

¹⁸F-DCFPyL produced at the extremes of the pH specification (4.5 and 7.0) for radiochemical concentrations up to 165 mCi/mL is expected to meet the RCP and chemical impurity product specifications for related substances over 10 hours post EOS. The lowest actual RCP value was 97.4% which was also at the highest pH (7.5) and highest radioactive concentration (162 mCi/mL). This study supports a example target 5.5 mg/mL, pH 5.5, ascorbic acid formulation and provides confirmation that products at or near the pH specification acceptance limits of 4.0 to 7.0 will meet the RCP specification of ≥95% at concentrations up to 165 mCi/mL.

### Ascorbic Acid Concentration Studies

The impact of ascorbic acid on the stability of ¹⁸F-DCFPyL was studied at the example target ascorbic acid level and at the limits of 3.0 and 7.0 mg/mL. Lots were prepared and samples were analyzed at 0, 2, 4, 6, and 10 hours for RCP and at 0 and 10 hours for pH. See Table 48 for a summary of the composition of the delivery solution vial, the collection vial and the FPV.

Lot 230130Pyl was prepared with 10 mg/mL pH 2.0 ascorbic acid in the collection vial and 3.3 mg/mL pH 5.5 ascorbic acid in the FPV. The radiochemical concentration of the product was 150 mCi/mL. The RCP was 99.0% at 0 hours and 96.7% at 10 hours, Table 49. No other radiochemical impurities were present at a reportable level; the T=10 hour chromatogram is provided in FIG. 31.

Lot 221026Pyl was prepared with 10 mg/mL pH 2.0 ascorbic acid in the collection vial and 10.0 mg/mL pH 5.5 ascorbic acid in the FPV The radiochemical concentration of the product was 133 mCi/mL. The RCP was 99.4% at 0 hours and 98.0% at 10 hours, Table 49. There were no reportable radiochemical impurities present throughout the study.

Lot 230131Pyl was prepared with 10 mg/mL pH 2.0 ascorbic acid in the collection vial and 14.0 mg/mL pH 5.5 ascorbic acid in the FPV. The radiochemical concentration of the product was 165 mCi/mL. The RCP was 99.0% at 0 hours and 97.3% at 10 hours, Table 49. There were no reportable radiochemical impurities present throughout the study. The T=10 hour chromatogram is provided in FIG. 32.

TABLE 25
Studies of PYLARIFY product at different ascorbic acid levels

| Vial | Lot 230130Pyl | Lot 221025Pyl | Lot 230131Pyl |
|---|---|---|---|
| Collection vial | 35 mL of 10.0 mg/mL pH 2.0 | 35 mL of 10.0 mg/mL pH 2.0 | 35 mL of 10.0 mg/mL pH 2.0 |
| (Position I, | ascorbic acid in 0.9% | ascorbic acid in 0.9% | ascorbic acid in 0.9% |
| FIG. 13) | Sodium Chloride, USP | Sodium Chloride, USP | Sodium Chloride, USP |
| Delivery | Fill: 20 mL of 0.9% | Fill: 20 mL of 0.9% | Fill: 20 mL of 0.9% |
| solution vial | Sodium Chloride, USP | Sodium Chloride, USP | Sodium Chloride, USP |
| (Position F, | Delivery: 16 mL to FPV | Delivery: 16 mL to FPV | Delivery: 16 mL to FPV |
| FIG. 13) | | | |
| FPV | 20 mL of 6.0 mg/mL | 20 mL of 10.0 mg/mL | 20 mL of 14.0 mg/mL |
| | ascorbic acid, | ascorbic acid, | ascorbic acid, |
| | pH 5.5 in 0.9% | pH 5.5 in 0.9% | pH 5.5 in 0.9% |
| | Sodium Chloride, USP | Sodium Chloride, USP | Sodium Chloride, USP |
| Product | 3.3 mg/mL, pH 5.5^{a} | 5.6 mg/mL, pH 5.5^{b} | 7.8 mg/mL, pH 5.5^{c} |
| ascorbic acid | | | |
| concentration | | | |
| and pH | | | |

^{a}16 mL 0.9% Sodium Chloride, USP, plus 20 mL of 6 mg/mL ascorbic acid = (20 mL × 6 mg/mL)/36 mL = 3.3 mg/mL ascorbic acid
^{b}16 mL 0.9% Sodium Chloride, USP, plus 20 mL of 10 mg/mL ascorbic acid = (20 mL × 10 mg/mL)/36 mL = 5.6 mg/mL ascorbic acid
^{c}16 mL 0.9% Sodium Chloride, USP, plus 20 mL of 14 mg/mL ascorbic acid = (20 mL × 14 mg/mL)/36 mL = 7.8 mg/mL ascorbic acid

TABLE 26
Data for PYLARIFY produced at different ascorbic acid concentrations

| | Lot 230130Pyl | | Lot 221025Pyl | | Lot 230131PyL | |
|---|---|---|---|---|---|---|
| Timepoint (hr) | T = 0 | T = 10 | T = 0 | T = 10 | T = 0 | T = 10 |
| pH | 6.0 | 6.4 | 5.9 | 6.3 | 5.9 | 6.0 |

| Starting activity | 15.6 | 14.7 | 16.4 |
|---|---|---|---|
| mCi/mL | 150 | 133 | 165 |
| Collection Vial | 10.0 mg/mL pH 2.0 ascorbic | 10.0 mg/mL pH 2.0 ascorbic | 10.0 mg/mL pH 2.0 ascorbic |
| | acid in 0.9% Sodium | acid in 0.9% Sodium | acid in 0.9% Sodium |
| | Chloride, USP | Chloride, USP | Chloride, USP |
| Delivery | 0.9% Sodium Chloride, USP | 0.9% Sodium Chloride, USP | 0.9% Sodium Chloride, USP |
| Solution Vial | | | |
| FPV | 6.0 mg/mL ascorbic acid, pH 4.5 | 10.0 mg/mL ascorbic acid, pH 5.5 | 14.0 mg/mL ascorbic acid, pH 7.0 |
| | in 0.9% Sodium Chloride, USP | in 0.9% Sodium Chloride, USP | in 0.9% Sodium Chloride, USP |
| Product | 3.3 mg/mL | 5.6 mg/mL | 7.8 mg/mL |
| ascorbic acid | | | |
| concentration | | | |

| Timepoint (hr) | T = 0^{a} | T = 2 | T = 4 | T = 6 | T = 10 | T = 0 | T = 2 | T = 4 | T = 6 | T = 10 | T = 0 | T = 2 | T = 4 | T = 6 | T = 10 |
|---|---|---|---|---|---|---|---|---|---|---|---|---|---|---|---|
| RCP^{b} | 99.0 | 98.0 | 97.4 | 96.9 | 96.7 | 98.9 | 98.2 | 98.1 | 97.9 | 97.4 | 99.1 | 98.2 | 97.8 | 97.3 | 97.3 |
| 18F^{b} | 1.05 | 2.02 | 2.58 | 3.11 | 3.35 | 0.71 | 1.33 | 1.89 | 2.10 | 2.08 | 0.94 | 1.84 | 2.24 | 2.71 | 2.75 |
| RT 6.0 min^{b} | nd^{c} | nd^{c} | nd^{c} | nd^{c} | nd^{c} | 0.42 | 0.52 | nd^{c} | nd^{c} | nd^{c} | nd^{c} | nd^{c} | nd^{c} | nd^{c} | nd^{c} |
| RT 9.8 min^{b} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} |
| RT 10.6 min^{b} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} | nd^{c} |

^{a}All times are in hours,
^{b}All values are in area %,
^{c}nd = not detected

¹⁸F-DCFPyL produced at the ascorbic acid example target and specification levels (3.3, 5.5 and 7.8 mg/mL) for radiochemical concentrations up to 165 mCi/mL is expected to meet the RCP and chemical impurity product specifications for related substances over 10 hours post EOS. The lowest actual RCP value was 96.7% for the lot having a 3.3 mg/mL ascorbic acid concentration and a radioactive concentration of 150 mCi/mL. This study supports an example target 5.6 mg/mL, pH 5.5, ascorbic acid formulation and provides confirmation that product at or near the ascorbic acid specification acceptance limits of 3.3 to 7.8 mg/mL will meet the RCP specification of ≥95% at concentrations up to 165 mCi/mL.

### Study on Effect of pH on ¹⁸F-DCFPyL Retention on the HLB Cartridge

The goal of this study was to characterize the impact of pH on the retention of ¹⁸F-DCFPyL on the HLB solvent exchange cartridge. Three separate 10 mg/mL ascorbic acid solutions were prepared. The pH of one solution was adjusted to 2 and a second to 4 by adding 1N HCl. A third solution was used without adjusting its native pH of 7.2. At this pH the solution will be mainly sodium ascorbate. Using a BD syringe, 10 mL of each solution was spiked with ¹⁹F-DCFPyL (50 μg) by adding 0.5 mL of a 100 μg/mL stock solution. Each spiked solution was loaded onto an HLB cartridge at a rate of approximately 2 mL/min and the solution collected after the HLB column is identified as the load solution. Each HLB cartridge was then rinsed with 10 mL water at a rate of approximately 2 mL/min and the solution collected after the HLB column is identified as the wash solution. Each HLB cartridge was then individually eluted with 1 mL of ethanol, the solution collected after the HLB column is identified as the product sample. The load solution and wash solution samples were analyzed using the drug product chemical impurity assay after a 1 to 10 mL dilution with saline, while the ethanolic product samples were analyzed following a 0.5 to 10 mL dilution with saline.

The ¹⁹F-DCFPyL concentration of the load and recovery samples varied as a function of pH. At a pH of 7.2 the ¹⁹F-DCFPYL was not meaningfully retained by the HLB cartridge and was recovered in the load sample (collected after the column). A small amount of ¹⁹F-DCFPyL was recovered in the wash samples (3.8 μg/mL) while there was no recovery in the product samples. At pH 4, a portion of the ¹⁹F-DCFPyL was recovered in the load sample (approximately 31%; <1 μg) and a larger amount in the product sample (approximately 68% of the total amount recovered from all samples). At pH 2, ¹⁹F-DCFPYL was not detected in the load or wash samples. The ¹⁹F-DCFPYL was recovered in the product solution. Due to the overall recovery (concentration) being significantly less than the amount applied to the HLB column a second ethanol elution volume was used. An additional recovery of ¹⁹F-DCFPyL was observed (approximately 25% of the amount recovered in the first elution). The results are summarized in Table 50.

The volumes of the different solutions were determined to allow calculation of the mass recoveries. The results clearly indicate that there is only partial retention of ¹⁹F-DCFPyL on the HLB cartridge at pH 4 and higher. At lower pH (e.g. pH 2) retention on the HLB column was apparent. These studies indicate the pH of the collection vial solution (the solution loaded onto the HLB solvent exchange cartridge) can be set to pH 2.

TABLE 27
Recoveries from the solvent exchange HLB cartridge
for Load, Rinse and Product samples

| Sample | μg of ¹⁹F-DCFPyL recovered (%) |
|---|---|
| pH 7 Control | 43.759 |
| HLB pH 7 Load | 38.239 (87.4%) |
| HLB Saline pH 7 Rinse | Not Detected (0.0%) |
| HLB pH 7 Product | Not Detected (0.0%) |
| Combined recovery for | 87.4% |
| pH 7 samples | |
| pH 4 Control | 47.665 |
| HLB pH 4 Load | 10.504 (22.0%) |
| HLB pH 4 Rinse | Not Detected (0.0%) |
| HLB pH 4 Product | 27.989 (57.7%) |
| Combined recovery for | 80.8% |
| pH 4 samples | |
| pH 2 Control | 50.976 |
| HLB pH 2 Load | Not Detected (0.0%) |
| HLB pH 2 Rinse | Not Detected (0.0%) |
| HLB pH 2 Product, first | 35.423 (69.5%) |
| Ethanol elution | |
| HLB pH 2 Product, second | 10.946 (21.5%) |
| Ethanol elution | |
| HLB pH 2 Product, third | 3.629 (7.1%) |
| Ethanol elution | |
| Combined recovery for | 69.5% |
| pH 2 one Ethanol elution | |
| Combined recovery for | 91.0% |
| pH 2 two Ethanol elutions | |
| Combined recovery for | 98.1% |
| pH 2 three Ethanol elutions | |

### Investigation of Ascorbic Acid Chemical Impurity

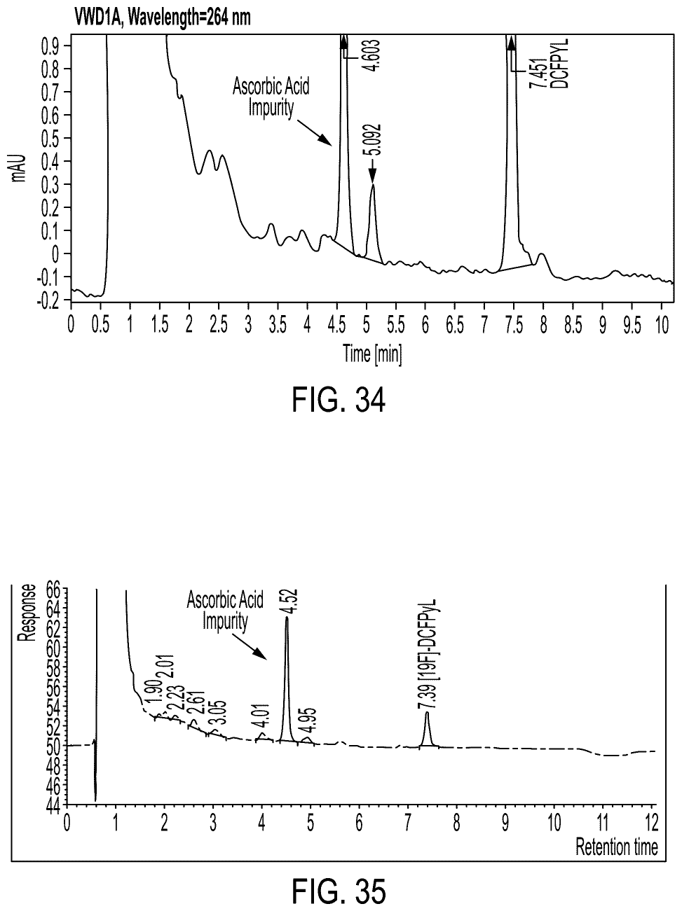

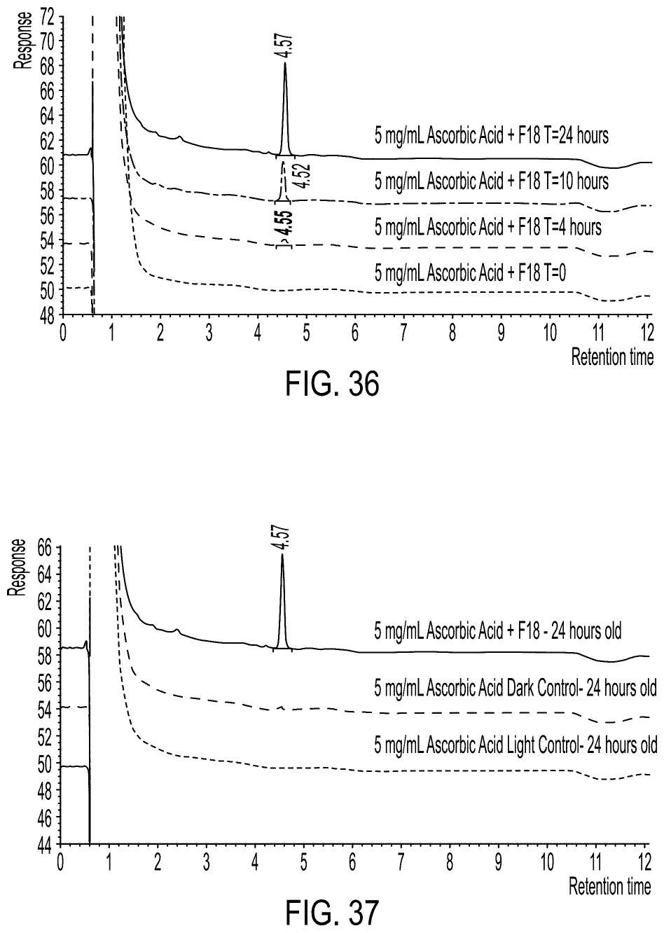

During a development lot of [¹⁸F]-DCFPyL prepared at 163.0 mCi/mL, a chemical impurity was observed at a retention time of approximately 4.6 minutes on the UV trace at 0.9 μg/mL, FIG. 34. Since the peak has not been previously observed in preparations in the absence of ascorbic acid it is likely ascorbic acid related and was further investigated. A decayed sample from the lot produced was analyzed further and confirmed the presence of the peak, FIG. 35. To exclude the impurity involved PLYLARIFY and demonstrate the formation from ascorbic acid, free ¹⁸F (129 mCi/mL) was incubated for different time periods in a 5 mg/mL, pH 6, ascorbic acid solution. The sample was assayed for chemical impurities at T=0, 4, 10 and 24 hours when held inverted at ambient conditions. The 4.6 min RT peak as assessed by HPLC UV detection was not present at T=0 hours, and increased in a time related manner through 24 hr with an approximate concentration of 1.75 μg/mL (assuming a relative response factor of 1 to the [¹⁹F]-DCFPyL standard) at T=24 hours (see FIG. 36). The impurity only formed with radioactivity exposure but not when stored in light or dark at ambient conditions (see FIG. 37). Since the ascorbic acid related UV impurity peak can be formed in the absence of PYLARIFY by incubation with ¹⁸F, does not match a radioactive peak and is not formed by exposure to light or dark it is concluded it is formed from ascorbic acid by radiolysis.

The degradation of ascorbic acid has been studied (Analytical Biochemistry, 265, 238-245 (1998), Journal of Chromatography A, 881 299-307 (2000). Oxidation of ascorbic acid to dehydroascorbic acid (DHA) is a key species in ascorbate degradation. Dehydroascorbic acid was obtained from Apollo Scientific (Catalog #BIB6039), and analyzed using a HPLC UV chemical impurity method. The DHA did not elute at the same time as the impurity.

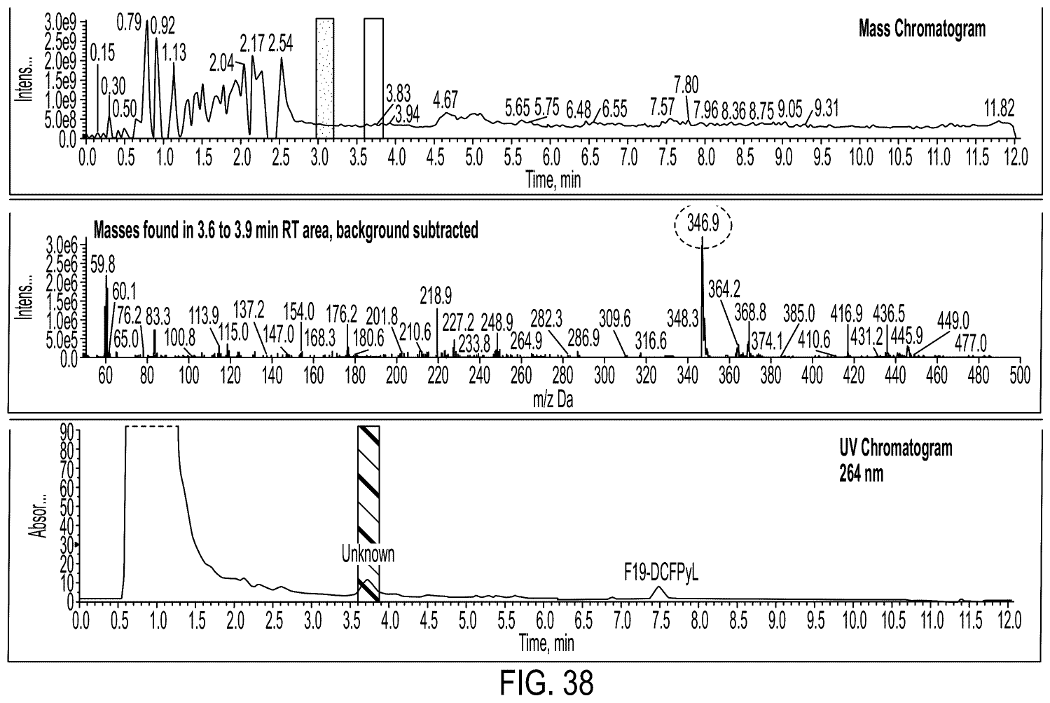

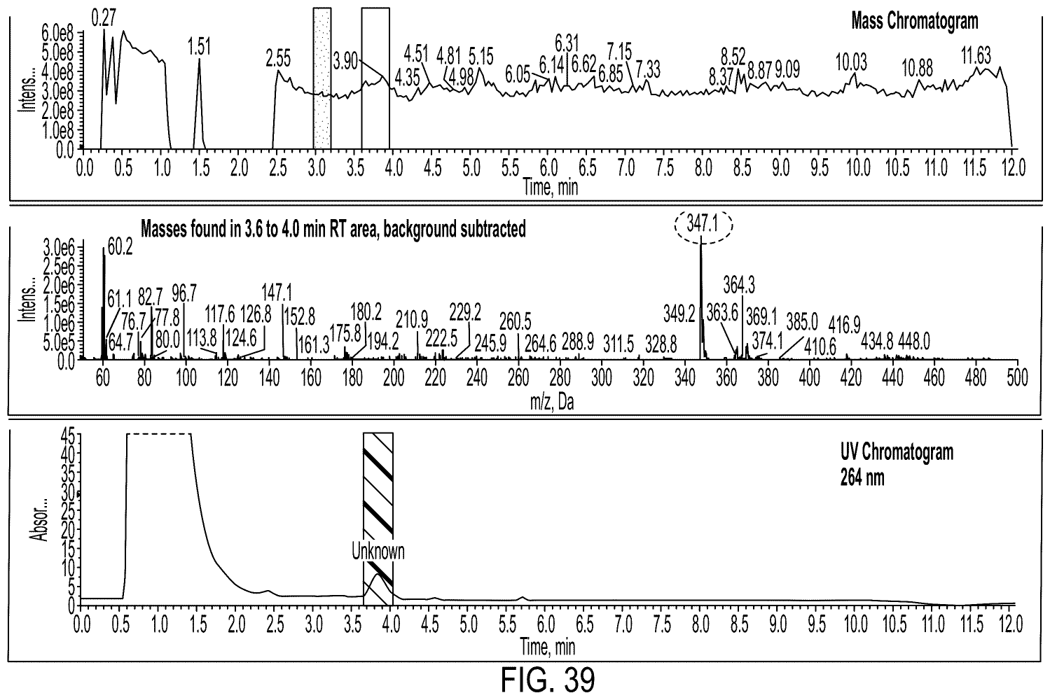

HPLC/MS analysis was performed on the decayed development sample. For mass spec compatibility, 0.1% Formic Acid was used as the modifier instead of 0.1% TFA. With the modified mobile phase, the decayed sample had a UV peak with retention time of approximately 3.8 min at 264 nm in addition to the [¹⁹F]-DCFPyL peak at approximately 7.5 min. The mass spec chromatogram at the corresponding retention time showed a peak with m/z 346.9, FIG. 38. HPLC/MS analysis was also performed on the sample spiked with F¹⁸ at approximately 120 mCi/mL into a 5 mg/mL, pH 6, ascorbic acid solution at T=24 hours (see FIG. 39).

The 5 mg/mL ascorbic acid+F¹⁸ solution sample showed a UV peak with retention time of approximately 3.8 min at 264 nm. The mass spec chromatogram at the corresponding retention time showed a peak with m/z 347.1, confirming that the mass of the impurity peak formed from ascorbic acid is the same as the impurity found in the development lot. The m/z 175 of DHA was not detected in this sample. The m/z of approximately 347 was confirmed in two separate samples, one decayed PYLARIFY sample and the other generated by incubation of ascorbic acid with F¹⁸.

The unknown impurity peak from the decayed PYLARIFY sample was fragmented using LC/MS/MS over a range of collision energies in both positive and negative modes. Literature suggests a common degradant of ascorbic acid is the dehydroascorbic acid monomer (DHA) which may have the potential to combine with another DHA molecule or an alternate form of ascorbic acid. The masses of the fragments observed from the 347 m/z impurity did not match any of the major fragments of the [¹⁹F]-DCFPyL standard, therefore, further confirming that the impurity is related to ascorbic acid and not PYLARIFY.

Configuration of Final Process Formulation with Ascorbic Acid

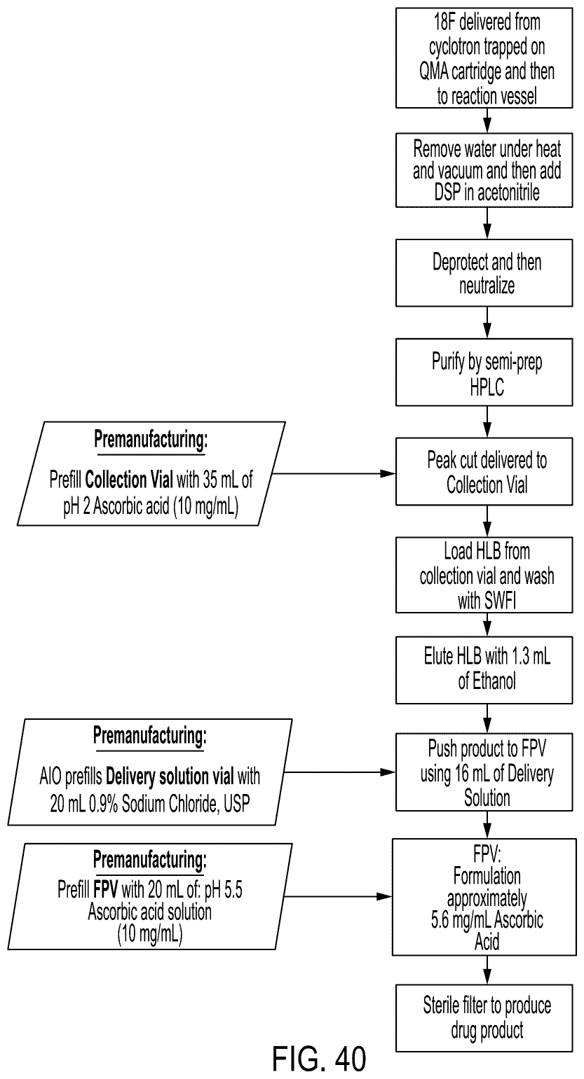

The data generated during the course of these studies led to the selection of an example formulation having 5.6 mg/mL ascorbic acid at pH 5.5 for PYLARIFY. It also led to the use of a 10 mg/mL ascorbic acid Solution pH 2 in the collection vial (Position I, FIG. 13). The final formulation can be obtained by delivering 1.3 mL ethanol followed by 15 mL of Saline into the FPV. The FPV can be preloaded with 20 mL of a 10 mg/mL ascorbic acid solution at pH 5.5. The final product volume can be 36 mL and thus has a 5.6 mg/mL ascorbic acid concentration. This formulation has demonstrated stability at RAC (Radioactive Concentration)≤165 mCi/mL. A flow diagram indicating where ascorbic acid is pre-loaded prior initiation of [¹⁸F]-DCFPyL production in order to protect the product at points of high radioactivity is shown in FIG. 40. In an embodiment of any one of the methods or compositions provided herein any one or more or all of the foregoing steps and/or features can be included as part of the method or compositions, respectively.

Studies were performed to develop a PYLARIFY formulation having a higher radioactive concentration. The primary degradation of the [¹⁸F]-DCFPyL is radiolysis that releases ¹⁸F. Incorporation of 5 mg/mL, pH 6 ascorbate as ascorbic acid in the final product provided a 96% RCP at 10 hours. Adding 10 mg/mL pH 6 ascorbate as ascorbic acid to the collection vial that receives the peak cut from the semi-preparative HPLC purification step, in addition to loading the final product vial with 5 mg/mL pH 6 ascorbic acid, provided a product having a 99% RCP at 4 hours, however there was a significant portion (approximately 40%) of the product that was not retained on the SepPak. Lowering the pH of the ascorbate in the collection vial to pH 2 while maintaining the 5 mg/mL ascorbate at pH 6 in the FPV provided additional stabilization giving a 98% RCP at 10 hours while retaining >98% of the product on the SepPak. In an embodiment of any one of the methods or compositions provided herein any one or more or all of the foregoing steps and/or features can be included as part of the method or compositions, respectively.

The choice of pH 2 for the collection vial was driven by the improved retention of the [¹⁸F]-DCFPyL by the HLB cartridge 98% versus 81% at pH 2 and 4, respectively. The optimal pH for the product to bind to the HLB was pH 2, therefore the pH 2 ascorbic acid solution was selected for prefilling the collection vial. Ascorbic acid at 5 or 10 mg/mL is sufficient to achieve stabilization, however for ease of preparation, a stock ascorbic acid solution at 10 mg/mL was chosen which can then used after pH adjustment to prefill the FPV vial or collection vial. Supplying a kit containing the ascorbic acid solutions would be feasible using a 5 mg/mL ascorbic acid. In an embodiment of any one of the methods or compositions provided herein any one or more or all of the foregoing steps and/or features can be included as part of the method or compositions, respectively.

Finally, using 5 mg/mL ascorbate at pH 6 as the 20 mL FPV prefill and 6 mg/mL pH 6 ascorbate (16 mL) as the formulation delivery solution for the HLB instead of saline did not provide additional stabilization, 98% RCP at 4 hours when compared to the use of saline for formulation delivery from the HLB. A slight increase in the pH was observed over the 10 hours during the stability determinations which indicates a product produced at pH 6 or 7 could be close to the upper specification limit of 7. Based on the observed pH change during stability studies, the target pH was changed from 6 to 5.5 to ensure the product would be well within the upper specification limit of 7 at 10 hours. These studies established example target solutions 16 mL of 5.6 mg/mL ascorbate at pH 5.5 in the FPV, 35 mL of 10 mg/mL pH 2 ascorbate in the collection vial, and 0.9% Sodium Chloride as the HLB formulation delivery solution. In an embodiment of any one of the methods or compositions provided herein any one or more or all of the foregoing steps and/or features can be included as part of the method or compositions, respectively.

Studies were then performed at three ascorbate levels in the FPV while maintaining a 10 mg/mL of pH 2 ascorbic acid in the collection vial. The results for 3.3, 5.6, and 7.8 mg/mL ascorbic acid had % RCPs of 97, 98, and 97 at 10 hours, respectively. Studies were also performed at the pH specification extremes and at the target pH to confirm the product meets product specifications across the pH specification range. The results were 98, 97, and 97% RCP at pH 4.5, 5.5 and 7.0 at 10 hours, respectively. These studies established the example proposed product preparation solutions produce a [¹⁸F]-DCFPyL that meets the specifications at the extremes of ascorbate concentration and pH. The data generated during the course of these studies led to the selection of an example final formulation having 5.6 mg/mL Ascorbic Acid at pH 5.5 for PYLARIFY. This formulation has demonstrated stability at RAC (Radioactive Concentration)≤160 mCi/mL. In an embodiment of any one of the methods or compositions provided herein any one or more or all of the foregoing steps and/or features can be included as part of the method or compositions, respectively.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A composition, comprising [¹⁸F]DCFPyL of the formula: 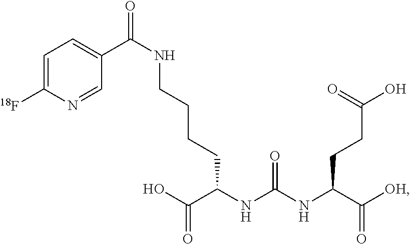 [SMILES: O=C(O)CC[C@H](NC(=O)N[C@@H](CCCCNC(=O)c1ccc([18F])nc1)C(=O)O)C(=O)O]
in a solution, optionally an ethanol solution, which solution further comprises ascorbic acid at a concentration of 3 to 15 mg/mL, 3 to 8 mg/mL, or 5 to 15 mg/mL, and wherein the pH of said solution is within the range of 4 to 7.5.

2. The composition of claim 1, wherein the ascorbic acid concentration is 3.5 to 8 mg/mL, 3.5 to 7.5 mg/mL, 3.5 to 7 mg/mL, 4 to 8 mg/mL, 4 to 7.5 mg/mL, 4 to 7 mg/mL, 4.5 to 8 mg/mL, 4.5 to 7.5 mg/mL, 4.5 to 7 mg/mL, 5 to 8 mg/mL, 5 to 7.5 mg/mL, 5 to 7 mg/mL, 5.5 to 8 mg/mL, 5.5 to 7.5 mg/mL, 5.5 to 7 mg/mL, 6 to 8 mg/mL, 6 to 7.5 mg/mL, 6 to 7 mg/mL, 6.5 to 8 mg/mL, 6.5 to 7.5 mg/mL, 6.5 to 7 mg/mL, 7 to 8 mg/mL, 7 to 7.5 mg/mL, or 7.5 to 8 mg/mL.

3-4. (canceled)

5. The composition of claim 1, wherein the pH of the solution is in the range of 3.5 to 7.5, 4 to 7.5, 4.5 to 7.5, 5 to 7.5, 5.5 to 7.5, 6 to 7.5, 6.5 to 7.5 or 7 to 7.5.

6. The composition of claim 1, wherein the pH of the solution is in the range of 3.5 to 7, 4 to 7, 4 to 6.5, 4 to 6, 4 to 5.5, 4 to 5 or 4 to 4.5.

7. The composition of claim 1, wherein the pH of the solution is in the range of 4.5 to 7, 5 to 6.5 or 5.5 to 6.

8. The composition of claim 1, wherein the solution has a radiochemical purity (RCP) that is at least 90% at 10 hours post synthesis.

9. The composition of claim 8, wherein the solution has a RCP that is at least 91%, 92%, 93%, 94% or 95% at 10 hours post synthesis.

10. The composition of claim 1, wherein the solution has a RCP that is at least 95% at end of synthesis (EOS).

11. The composition of claim 10, wherein the solution has a RCP that is at least 96%, 97%, 98% or 99% at EOS.

12. The composition of claim 1, wherein the solution has a radioactive concentration that is at least 80 mCi/mL at EOS.

13. The composition of claim 12, wherein the solution has a radioactive concentration that is at least 85 mCi/mL, 90 mCi/mL, 95 mCi/mL, 100 mCi/mL, 105 mCi/mL, 110 mCi/mL, 115 mCi/mL, 120 mCi/mL, 125 mCi/mL, 130 mCi/mL, 135 mCi/mL, 140 mCi/mL, 145 mCi/mL, 150 mCi/mL, 160 mCi/mL, 165 mCi/mL, 166 mCi/mL or 167 mCi/mL at EOS.

14. The composition of claim 12, wherein the solution has a radioactive concentration that is equal to or less than 155 mCi/mL, 160 mCi/mL, 161 mCi/mL, 162 mCi/mL, 163 mCi/mL, 164 mCi/mL, 165 mCi/mL, 166 mCi/mL, 167 mCi/mL, 168 mCi/mL, 169 mCi/mL or 170 mCi/mL at EOS.

15. The composition of claim 1, wherein the solution has less than 5% free ¹⁸F.

16. The composition of claim 15, wherein the solution has less the 4.5% free ¹⁸F.

17. The composition of claim 16, wherein the solution has less the 4% free ¹⁸F.

18. The composition of claim 17, wherein the solution has less the 3.5% free ¹⁸F.

19. The composition of claim 18, wherein the solution has less the 3% free ¹⁸F.

20. The composition of claim 1, wherein the solution comprises ethanol and is less than 30% ethanol w/v.

21. The composition of claim 20, wherein the solution is less than 25% ethanol w/v.

22. The composition of claim 21, wherein the solution is less than 20% ethanol w/v.

23. The composition of claim 22, wherein the solution is less than 15% ethanol w/v.

24. The composition of claim 23, wherein the solution is less than 10% ethanol w/v.

25. The composition of claim 24, wherein the solution is less than 5% ethanol w/v.

26. The composition of claim 20, wherein the solution is at least 3% ethanol w/v.

27. The composition of claim 1, wherein the solution comprises a total of unknown impurities of less than or equal to 2 μg/mL.

28. The composition of claim 27, wherein the solution comprises a total of unknown impurities of less than or equal to 1.5 μg/mL.

29. (canceled)

30. The composition of claim 1, wherein the solution comprises an ascorbic acid-related impurity of ≤5.0 μg/mL.

31. (canceled)

32. The composition of claim 1, wherein the solution comprises less than or equal to ≤0.04% w/v acetonitrile.

33-115. (canceled)

116. A composition comprising the components at the respective quantities provided in Table 32.

117-130. (canceled)
